SEQUENCE LISTING (216 sequences)

SEQ ID NO: 1 - protein, 9 aa, Unknown
  Description of Unknown ′LAGLIDADG′ family peptide motif
LAGLIDADG

SEQ ID NO: 2 - DNA, 42 bp, Artificial Sequence
  Description of Artificial Sequence Synthetic oligonucleotide
caacgctcagtagatgttttggtccacatatttaaccttttg

SEQ ID NO: 3 - DNA, 9 bp, Artificial Sequence
  Description of Artificial Sequence Synthetic oligonucleotide
gcgtgggcg

SEQ ID NO: 4 - protein, 34 aa, Xanthomonas sp.
LTPEQVVAIASNIGGKQALETVQALLPVLCQAHG

SEQ ID NO: 5 - protein, 34 aa, Xanthomonas sp.
LTPDQVVAIASEGGGKQALETVQRLLPVLCQAHG

SEQ ID NO: 6 - protein, 34 aa, Xanthomonas sp.
LTPEQVVAIASNIGGKQALETVQRLLPVLCQAHG

SEQ ID NO: 7 - protein, 34 aa, Ralstonia solanacearum
LTPQQWAIASNTGGKRALEAVCVQLPVLRAAPYR

SEQ ID NO: 8 - protein, 34 aa, Ralstonia solanacearum
LSTEQWAIASNKGGKQALEAVKAHLLDLLGAPYV

SEQ ID NO: 9 - protein, 35 aa, Ralstonia solanacearum
LDTEQVVAIASHNGGKQALEAVKADLLDLRGAPYA

SEQ ID NO: 10 - protein, 34 aa, Artificial Sequence
  Description of Artificial Sequence Synthetic polypeptide
LXXXQVVAIASHDGGKQALXTXQRLLPVLCQXHG

SEQ ID NO: 11 - protein, 16 aa, Artificial Sequence
  Description of Artificial Sequence Synthetic peptide
KRXXXXXXXXXXKKKL

SEQ ID NO: 12 - protein, 7 aa, Artificial Sequence
  Description of Artificial Sequence Synthetic peptide
PKKKRKV

SEQ ID NO: 13 - protein, 9 aa, Artificial Sequence
  Description of Artificial Sequence Synthetic peptide
PKKNRLRRP

SEQ ID NO: 14 - protein, 8 aa, Artificial Sequence
  Description of Artificial Sequence Synthetic peptide
PLLKKIKQ

SEQ ID NO: 15 - DNA, 22 bp, Artificial Sequence
  Description of Artificial Sequence Synthetic primer
ggaagaagtggctgatctcagc

SEQ ID NO: 16 - DNA, 20 bp, Artificial Sequence
  Description of Artificial Sequence Synthetic primer
cagaccgcttctgcgttctg

SEQ ID NO: 17 - DNA, 40 bp, Artificial Sequence
  Description of Artificial Sequence Synthetic primer
gctaaagattttgaaaaggcatggaagaagcattttaaag

SEQ ID NO: 18 - DNA, 40 bp, Artificial Sequence
  Description of Artificial Sequence Synthetic primer
ctttaaaatgcttcttccatgccttttcaaaatctttagc

SEQ ID NO: 19 - DNA, 54 bp, Artificial Sequence
  Description of Artificial Sequence Synthetic oligonucleotide
ctagacaacgctcagtagatgttttcttgggtctaccgtttcccacgccgcatg

SEQ ID NO: 20 - DNA, 46 bp, Artificial Sequence
  Description of Artificial Sequence Synthetic oligonucleotide
cggcgtgggaaacggtagacccaagaaaacatctactgagcgttgt

SEQ ID NO: 21 - DNA, 54 bp, Artificial Sequence
  Description of Artificial Sequence Synthetic oligonucleotide
ctagagcccgtagtaatgacatggccttgggaaatccctttcccacgccgcatg

SEQ ID NO: 22 - DNA, 46 bp, Artificial Sequence
  Description of Artificial Sequence Synthetic oligonucleotide
cggcgtgggaaagggatttcccaaggccatgtcattactacgggct

SEQ ID NO: 23 - DNA, 28 bp, Artificial Sequence
  Description of Artificial Sequence Synthetic primer
ccgcggatccattactaggctttttacc

SEQ ID NO: 24 - DNA, 34 bp, Artificial Sequence
  Description of Artificial Sequence Synthetic primer
ccgcggatccaccaccattactaggctttttacc

SEQ ID NO: 25 - DNA, 40 bp, Artificial Sequence
  Description of Artificial Sequence Synthetic primer
ccgcggatccaccaccaccaccattactaggctttttacc

SEQ ID NO: 26 - DNA, 33 bp, Artificial Sequence
  Description of Artificial Sequence Synthetic primer
ccgcggatcctttaatattactaggctttttac

SEQ ID NO: 27 - DNA, 39 bp, Artificial Sequence
  Description of Artificial Sequence Synthetic primer
ccgcggatccaccacctttaatattactaggctttttac

SEQ ID NO: 28 - DNA, 45 bp, Artificial Sequence
  Description of Artificial Sequence Synthetic primer
ccgcggatccaccaccaccacctttaatattactaggctttttac

SEQ ID NO: 29 - DNA, 35 bp, Artificial Sequence
  Description of Artificial Sequence Synthetic primer
ccgcggatcctgaaatctttttaatattactaggc

SEQ ID NO: 30 - DNA, 41 bp, Artificial Sequence
  Description of Artificial Sequence Synthetic primer
ccgcggatccaccacctgaaatctttttaatattactaggc

SEQ ID NO: 31 - DNA, 33 bp, Artificial Sequence
  Description of Artificial Sequence Synthetic primer
gccgccatgggtaaaagcggaatttatcagatt

SEQ ID NO: 32 - DNA, 28 bp, Artificial Sequence
  Description of Artificial Sequence Synthetic primer
ccgcggatccgtttttcggtttacgacc

SEQ ID NO: 33 - DNA, 34 bp, Artificial Sequence
  Description of Artificial Sequence Synthetic primer
ccgcggatccaccaccgtttttcggtttacgacc

SEQ ID NO: 34 - DNA, 40 bp, Artificial Sequence
  Description of Artificial Sequence Synthetic primer
ccgcggatccaccaccaccaccgtttttcggtttacgacc

SEQ ID NO: 35 - DNA, 31 bp, Artificial Sequence
  Description of Artificial Sequence Synthetic primer
ccgcggatccacgagagtttttcggtttacg

SEQ ID NO: 36 - DNA, 37 bp, Artificial Sequence
  Description of Artificial Sequence Synthetic primer
ccgcggatccaccaccacgagagtttttcggtttacg

SEQ ID NO: 37 - DNA, 43 bp, Artificial Sequence
  Description of Artificial Sequence Synthetic primer
ccgcggatccaccaccaccaccacgagagtttttcggtttacg

SEQ ID NO: 38 - DNA, 34 bp, Artificial Sequence
  Description of Artificial Sequence Synthetic primer
ccgcggatccgataaccggacgagagtttttcgg

SEQ ID NO: 39 - DNA, 40 bp, Artificial Sequence
  Description of Artificial Sequence Synthetic primer
ccgcggatccaccaccgataaccggacgagagtttttcgg

SEQ ID NO: 40 - DNA, 33 bp, Artificial Sequence
  Description of Artificial Sequence Synthetic primer
gccgccatgggtaaatctggtgtttacaaaatc

SEQ ID NO: 41 - DNA, 28 bp, Artificial Sequence
  Description of Artificial Sequence Synthetic primer
cttgggtctaccgttcccacgccgcatg

SEQ ID NO: 42 - DNA, 28 bp, Artificial Sequence
  Description of Artificial Sequence Synthetic primer
catgcggcgtgggaacggtagacccaag

SEQ ID NO: 43 - DNA, 27 bp, Artificial Sequence
  Description of Artificial Sequence Synthetic primer
cttgggtctaccgtcccacgccgcatg

SEQ ID NO: 44 - DNA, 27 bp, Artificial Sequence
  Description of Artificial Sequence Synthetic primer
catgcggcgtgggacggtagacccaag

SEQ ID NO: 45 - DNA, 30 bp, Artificial Sequence
  Description of Artificial Sequence Synthetic primer
gccttgggaaatcccttcccacgccgcatg

SEQ ID NO: 46 - DNA, 30 bp, Artificial Sequence
  Description of Artificial Sequence Synthetic primer
catgcggcgtgggaagggatttcccaaggc

SEQ ID NO: 47 - DNA, 29 bp, Artificial Sequence
  Description of Artificial Sequence Synthetic primer
gccttgggaaatccctcccacgccgcatg

SEQ ID NO: 48 - DNA, 29 bp, Artificial Sequence
  Description of Artificial Sequence Synthetic primer
catgcggcgtgggagggatttcccaaggc

SEQ ID NO: 49 - DNA, 40 bp, Artificial Sequence
  Description of Artificial Sequence Synthetic primer
cagaaacagctggtttaataacatcatcaccactaactcg

SEQ ID NO: 50 - DNA, 40 bp, Artificial Sequence
  Description of Artificial Sequence Synthetic primer
cgagttagtggtgatgatgttattaaaccagctgtttctg

SEQ ID NO: 51 - DNA, 54 bp, Artificial Sequence
  Description of Artificial Sequence Synthetic oligonucleotide
ctagacaacactcagtagatgttttcttgggtctaccgtttcccacgccgcatg

SEQ ID NO: 52 - DNA, 46 bp, Artificial Sequence
  Description of Artificial Sequence Synthetic oligonucleotide
cggcgtgggaaacggtagacccaagaaaacatctactgagtgttgt

SEQ ID NO: 53 - DNA, 54 bp, Artificial Sequence
  Description of Artificial Sequence Synthetic oligonucleotide
ctagaaaacactcagtagatgttttcttgggtctaccgtttcccacgccgcatg

SEQ ID NO: 54 - DNA, 46 bp, Artificial Sequence
  Description of Artificial Sequence Synthetic oligonucleotide
cggcgtgggaaacggtagacccaagaaaacatctactgagtgtttt

SEQ ID NO: 55 - protein, 4 aa, Artificial Sequence
  Description of Artificial Sequence Synthetic peptide
GGGG

SEQ ID NO: 56 - protein, 6 aa, Artificial Sequence
  Description of Artificial Sequence Synthetic 6xHis tag
HHHHHH

SEQ ID NO: 57 - protein, 8 aa, Artificial Sequence
  Description of Artificial Sequence Synthetic peptide
GSGSGSGS

SEQ ID NO: 58 - protein, 15 aa, Artificial Sequence
  Description of Artificial Sequence Synthetic peptide
GGGGSGGGGSGGGGS

SEQ ID NO: 59 - protein, 5 aa, Artificial Sequence
  Description of Artificial Sequence Synthetic peptide
GGGGS

SEQ ID NO: 60 - DNA, 14 bp, Homo sapiens
  modified_base (2)..(4): a, c, t, g, unknown or other
cnnngnwnnnnngw

SEQ ID NO: 61 - DNA, 42 bp, Unknown
  Description of Unknown Wild-type Tev-ryA target site oligonucleotide
caacgctcagtagatgttttcttgggtctaccgtcccacgcc

SEQ ID NO: 62 - DNA, 42 bp, Artificial Sequence
  Description of Artificial Sequence Synthetic oligonucleotide
gcccgtagtaatgacatggccttgggaaatccctcccacgcc

SEQ ID NO: 63 - DNA, 22 bp, Artificial Sequence
  Description of Artificial Sequence Synthetic oligonucleotide
caacgctcagtagatgttttct

SEQ ID NO: 64 - DNA, 22 bp, Artificial Sequence
  Description of Artificial Sequence Synthetic oligonucleotide
tgaaaacatctactgagcgttg

SEQ ID NO: 65 - DNA, 22 bp, Artificial Sequence
  Description of Artificial Sequence Synthetic oligonucleotide
gcccgtagtaatgacatggcct

SEQ ID NO: 66 - DNA, 22 bp, Artificial Sequence
  Description of Artificial Sequence Synthetic oligonucleotide
aggccatgtcattactacgggc

SEQ ID NO: 67 - DNA, 42 bp, Artificial Sequence
  Description of Artificial Sequence Synthetic oligonucleotide
aaacgctcagtagatgttttcttgggtctaccgtcccacgcc

SEQ ID NO: 68 - DNA, 42 bp, Artificial Sequence
  Description of Artificial Sequence Synthetic oligonucleotide
aaacactcagtagatgttttcttgggtctaccgtcccacgcc

SEQ ID NO: 69 - protein, 296 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 70 - protein, 298 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 71 - protein, 300 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 72 - protein, 298 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 73 - protein, 300 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 74 - protein, 302 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 75 - protein, 301 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 76 - protein, 303 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 77 - protein, 302 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 78 - protein, 304 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 79 - protein, 306 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 80 - protein, 304 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 81 - protein, 306 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 82 - protein, 308 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 83 - protein, 307 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 84 - protein, 309 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 85 - protein, 315 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 86 - protein, 317 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 87 - protein, 319 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 88 - protein, 317 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 89 - protein, 319 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 90 - protein, 321 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 91 - protein, 320 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 92 - protein, 322 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIGGGSGMEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 93 - protein, 321 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 94 - protein, 323 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 95 - protein, 325 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 96 - protein, 323 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 97 - protein, 325 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 98 - protein, 327 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRGGGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 99 - protein, 326 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 100 - protein, 328 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIGGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 101 - DNA, 43 bp, Artificial Sequence
  Description of Artificial Sequence Synthetic oligonucleotide
cnnngnnnnnnnnnnnnnnnnnnnnnnnnnnnnntcccacgcc

SEQ ID NO: 102 - DNA, 43 bp, Artificial Sequence
  Description of Artificial Sequence Synthetic oligonucleotide
ggcgtgggannnnnnnnnnnnnnnnnnnnnnnnnnnnncnnng

SEQ ID NO: 103 - DNA, 24 bp, Artificial Sequence
  Description of Artificial Sequence Synthetic oligonucleotide
gtagtagctnnnnnnggcgtggga

SEQ ID NO: 104 - DNA, 24 bp, Artificial Sequence
  Description of Artificial Sequence Synthetic oligonucleotide
tcccacgccnnnnnnagctactac

SEQ ID NO: 105 - DNA, 57 bp, Artificial Sequence
  Description of Artificial Sequence Synthetic oligonucleotide
caacgctcagtagatgttttcttgggtctacctgcatctcccactactgtaaaacac

SEQ ID NO: 106 - DNA, 49 bp, Artificial Sequence
  Description of Artificial Sequence Synthetic oligonucleotide
caacgctcagtagatgttttcttgtgcatctcccactactgtaaaacac

SEQ ID NO: 107 - DNA, 51 bp, Artificial Sequence
  Description of Artificial Sequence Synthetic oligonucleotide
caacgctcagtagatgttttcttgggtgcatctcccactactgtaaaacac

SEQ ID NO: 108 - DNA, 53 bp, Artificial Sequence
  Description of Artificial Sequence Synthetic oligonucleotide
caacgctcagtagatgttttcttgggtctgcatctcccactactgtaaaacac

SEQ ID NO: 109 - DNA, 55 bp, Artificial Sequence
  Description of Artificial Sequence Synthetic oligonucleotide
caacgctcagtagatgttttcttgggtctatgcatctcccactactgtaaaacac

SEQ ID NO: 110 - DNA, 57 bp, Artificial Sequence; identical to SEQ ID NO: 105
  Description of Artificial Sequence Synthetic oligonucleotide

SEQ ID NO: 111 - DNA, 59 bp, Artificial Sequence
  Description of Artificial Sequence Synthetic oligonucleotide
caacgctcagtagatgttttcttgggtctaccgttgcatctcccactactgtaaaacac

SEQ ID NO: 112 - DNA, 61 bp, Artificial Sequence
  Description of Artificial Sequence Synthetic oligonucleotide
caacgctcagtagatgttttcttgggtctaccgttttgcatctcccactactgtaaaacac

SEQ ID NO: 113 - DNA, 63 bp, Artificial Sequence
  Description of Artificial Sequence Synthetic oligonucleotide
caacgctcagtagatgttttcttgggtctaccgtttaatgcatctcccactactgtaaaacac

SEQ ID NO: 114 - protein, 1432 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 115 - protein, 1445 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 116 - protein, 1458 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 117 - protein, 1487 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 118 - protein, 1519 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 119 - protein, 1521 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 120 - protein, 1523 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGGGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 121 - protein, 1521 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 122 - protein, 1523 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGGGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 123 - protein, 1525 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGGGGGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 124 - protein, 1524 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 125 - protein, 1526 aa, Artificial Sequence
  Description of Artificial Sequence Synthetic polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISGGGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 126 - protein, 1432 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 127 - protein, 1442 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 128 - protein, 1456 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 129 - protein, 1476 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 130 - DNA, 55 bp, Artificial Sequence
  Description of Artificial Sequence Synthetic oligonucleotide
gcccgtagtaatgacatggccttgggaaattgcatctcccactactgtaaaacac

SEQ ID NO: 131 - DNA, 53 bp, Artificial Sequence
  Description of Artificial Sequence Synthetic oligonucleotide
gcccgtagtaatgacatggccttgggaatgcatctcccactactgtaaaacac

SEQ ID NO: 132 - DNA, 51 bp, Artificial Sequence
  Description of Artificial Sequence Synthetic oligonucleotide
gcccgtagtaatgacatggccttgggtgcatctcccactactgtaaaacac

SEQ ID NO: 133 - DNA, 49 bp, Artificial Sequence
  Description of Artificial Sequence Synthetic oligonucleotide
gcccgtagtaatgacatggccttgtgcatctcccactactgtaaaacac

SEQ ID NO: 134 - DNA, 41 bp, Artificial Sequence
  Description of Artificial Sequence Synthetic oligonucleotide
caacgnnnnnnnnnnnnnnnggtccacatatttaacctttt

SEQ ID NO: 135 - DNA, 41 bp, Artificial Sequence
  Description of Artificial Sequence Synthetic oligonucleotide
aaaaggttaaatatgtggaccnnnnnnnnnnnnnnncgttg

SEQ ID NO: 136 - DNA, 44 bp, Unknown
  Description of Unknown I-TevI binding domain oligonucleotide
tatcaacgctcagtagatgttttcttgggtctaccgtttaatat

SEQ ID NO: 137 - protein, 13 aa, Unknown
  Description of Unknown I-TevI binding domain peptide
QRSVDVFLGLPFN

SEQ ID NO: 138 - DNA, 44 bp, Unknown
  Description of Unknown I-TevI binding domain oligonucleotide
atattaaacggtagacccaagaaaacatctactgagcgttgata

SEQ ID NO: 139 - DNA, 41 bp, Artificial Sequence
  Description of Artificial Sequence Synthetic oligonucleotide
caacggaactgcgatcggctggtccacatatttaacctttt

SEQ ID NO: 140 - DNA, 41 bp, Artificial Sequence
  Description of Artificial Sequence Synthetic oligonucleotide
caacgctctcccgaactccaggtccacatatttaacctttt

SEQ ID NO: 141 - DNA, 41 bp, Artificial Sequence
  Description of Artificial Sequence Synthetic oligonucleotide
caacgctaagtcgatcagttggtccacatatttaacctttt

SEQ ID NO: 142 - DNA, 41 bp, Artificial Sequence
  Description of Artificial Sequence Synthetic oligonucleotide
caacgcaaccccgattttatggtccacatatttaacctttt

SEQ ID NO: 143 - DNA, 41 bp, Artificial Sequence
  Description of Artificial Sequence Synthetic oligonucleotide
caacggactgtggtgtggcgggtccacatatttaacctttt

SEQ ID NO: 144 - DNA, 41 bp, Artificial Sequence
  Description of Artificial Sequence Synthetic oligonucleotide
caacgatccgccgtcctcgaggtccacatatttaacctttt

SEQ ID NO: 145 - DNA, 41 bp, Artificial Sequence
  Description of Artificial Sequence Synthetic oligonucleotide
caacgtaccggcgatcgatcggtccacatatttaacctttt

SEQ ID NO: 146 - protein, 266 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIIDGTEYESATEASRQLNVVPATILHRIKSKNEKYSGYFYK

SEQ ID NO: 147 - protein, 245 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISCDGVIFDCAADAARHFKISSGLVTYRVKSDKWNWFYINA

SEQ ID NO: 148 - protein, 245 aa, Artificial Sequence
  Description of Artificial Sequence Synthetic polypeptide
MKSGIYQIKNTLNGKVYVGSAKDFEKRWKRHFKDLENGVHSSIKFQRSFNKHGNVFECSVLEEIPYEKDLIIERENFWIKELNSKINGYNIADASFGDVLSNHPLKEEIAKKRAETVKAKMLKLGPDGRKALYGKHGSKNGRWNPENHKFCKCGVRIPSSADTCGKCRKRSGENNPFFNHKHSEKTKTKLSEKMKGKKPANIKKISCDGIIFECAADAARHFEISSGLVTYRVKSDKWNWFYINA

SEQ ID NO: 149 - protein, 80 aa, Artificial Sequence
  Description of Artificial Sequence Synthetic polypeptide
NIADASFGDVLSNHPLKEEIAKKRAETVKAKMLKLGPDGRKALYGKHGSKNGRWNPENHKFCKCGVRIPSSADTCGKCRK

SEQ ID NO: 150 - protein, 80 aa, Artificial Sequence
  Description of Artificial Sequence Synthetic polypeptide
NIADATFGDTCSTHPLKEEIIKKRSETVKAKMLKLGPDGRKALYSKPGSKNGRWNPETHKFCKCGVRIQTSAYTCSKCRN

SEQ ID NO: 151 - protein, 82 aa, Artificial Sequence
  Description of Artificial Sequence Synthetic polypeptide
NISKSAYHGGDLTSYHPDKRNIILKRADSLKKVYLKMTSEEKAKRWQCVQGENNPMFGRKHTETTKLKISNHNKLYYSTHKN

SEQ ID NO: 152 - protein, 91 aa, Artificial Sequence
  Description of Artificial Sequence Synthetic polypeptide
MEPYACPVESCDRRFSQSGHLQRHIRIHTGQKPFQCRICMRNFSRSDALTRHIRTHTGEKPFACDICGRKFSDPGHLVRHTKIHLRQKQLV

SEQ ID NO: 153 - protein, 91 aa, Artificial Sequence
  Description of Artificial Sequence Synthetic polypeptide
MEPYACPVESCDRRFSQSSDLTRHIRIHTGQKPFQCRICMRNFSQSSSLVRHIRTHTGEKPFACDICGRKFSQSSSLVRHTKIHLRQKQLV

SEQ ID NO: 154 - protein, 1318 aa, Artificial Sequence
  Description of Artificial Sequence Synthetic polypeptide
MGDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPD

SEQ ID NO: 155 - protein, 1164 aa, Artificial Sequence
  Description of Artificial Sequence Synthetic polypeptide
MDPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNSGGKQALETVQRLLPVLCQAHGLTPEQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNSGGKQALETVQALLPVLCQAHGLTPEQVVAIASNSGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 156 - protein, 305 aa, Artificial Sequence
  Description of Artificial Sequence Synthetic polypeptide
MGSAYMSRRESINPWILTGFADAEGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVF

SEQ ID NO: 157 - DNA, 9 bp, Artificial Sequence
  Description of Artificial Sequence Synthetic oligonucleotide
tcccacgcc

SEQ ID NO: 158 - DNA, 9 bp, Artificial Sequence
  Description of Artificial Sequence Synthetic oligonucleotide
agctactac

SEQ ID NO: 159 - DNA, 25 bp, Artificial Sequence
  Description of Artificial Sequence Synthetic oligonucleotide
tgcatctccccctactgtacaccac

SEQ ID NO: 160 - DNA, 25 bp, Artificial Sequence
  Description of Artificial Sequence Synthetic oligonucleotide
tgcatctcccattactgtaaaacac

SEQ ID NO: 161 - DNA, 21 bp, Artificial Sequence
  Description of Artificial Sequence Synthetic oligonucleotide
tatataaacctaaccatcctc

SEQ ID NO: 162 - DNA, 22 bp, Artificial Sequence
  Description of Artificial Sequence Synthetic oligonucleotide
ggtccacatatttaaccttttg

SEQ ID NO: 163 - protein, 296 aa, Artificial Sequence; identical to SEQ ID NO: 69
  Description of Artificial Sequence Synthetic polypeptide

SEQ ID NO: 164 - protein, 298 aa, Artificial Sequence; identical to SEQ ID NO: 70
  Description of Artificial Sequence Synthetic polypeptide

SEQ ID NO: 165 - protein, 300 aa, Artificial Sequence; identical to SEQ ID NO: 71
  Description of Artificial Sequence Synthetic polypeptide

SEQ ID NO: 166 - protein, 298 aa, Artificial Sequence; identical to SEQ ID NO: 72
  Description of Artificial Sequence Synthetic polypeptide

SEQ ID NO: 167 - protein, 300 aa, Artificial Sequence; identical to SEQ ID NO: 73
  Description of Artificial Sequence Synthetic polypeptide

SEQ ID NO: 168 - protein, 302 aa, Artificial Sequence; identical to SEQ ID NO: 74
  Description of Artificial Sequence Synthetic polypeptide

SEQ ID NO: 169 - protein, 301 aa, Artificial Sequence; identical to SEQ ID NO: 75
  Description of Artificial Sequence Synthetic polypeptide

SEQ ID NO: 170 - protein, 303 aa, Artificial Sequence; identical to SEQ ID NO: 76
  Description of Artificial Sequence Synthetic polypeptide

SEQ ID NO: 171 - protein, 302 aa, Artificial Sequence; identical to SEQ ID NO: 77
  Description of Artificial Sequence Synthetic polypeptide

SEQ ID NO: 172 - protein, 304 aa, Artificial Sequence; identical to SEQ ID NO: 78
  Description of Artificial Sequence Synthetic polypeptide

SEQ ID NO: 173 - protein, 306 aa, Artificial Sequence; identical to SEQ ID NO: 79
  Description of Artificial Sequence Synthetic polypeptide

SEQ ID NO: 174 - protein, 304 aa, Artificial Sequence; identical to SEQ ID NO: 80
  Description of Artificial Sequence Synthetic polypeptide

SEQ ID NO: 175 - protein, 306 aa, Artificial Sequence; identical to SEQ ID NO: 81
  Description of Artificial Sequence Synthetic polypeptide

SEQ ID NO: 176 - protein, 308 aa, Artificial Sequence; identical to SEQ ID NO: 82
  Description of Artificial Sequence Synthetic polypeptide

SEQ ID NO: 177 - protein, 307 aa, Artificial Sequence; identical to SEQ ID NO: 83
  Description of Artificial Sequence Synthetic polypeptide

SEQ ID NO: 178 - protein, 309 aa, Artificial Sequence; identical to SEQ ID NO: 84
  Description of Artificial Sequence Synthetic polypeptide

SEQ ID NO: 179 - protein, 315 aa, Artificial Sequence; identical to SEQ ID NO: 85
  Description of Artificial Sequence Synthetic polypeptide

SEQ ID NO: 180 - protein, 317 aa, Artificial Sequence; identical to SEQ ID NO: 86
  Description of Artificial Sequence Synthetic polypeptide

SEQ ID NO: 181 - protein, 319 aa, Artificial Sequence; identical to SEQ ID NO: 87
  Description of Artificial Sequence Synthetic polypeptide

SEQ ID NO: 182 - protein, 317 aa, Artificial Sequence; identical to SEQ ID NO: 88
  Description of Artificial Sequence Synthetic polypeptide

SEQ ID NO: 183 - protein, 319 aa, Artificial Sequence; identical to SEQ ID NO: 89
  Description of Artificial Sequence Synthetic polypeptide

SEQ ID NO: 184 - protein, 321 aa, Artificial Sequence; identical to SEQ ID NO: 90
  Description of Artificial Sequence Synthetic polypeptide

SEQ ID NO: 185 - protein, 320 aa, Artificial Sequence; identical to SEQ ID NO: 91
  Description of Artificial Sequence Synthetic polypeptide

SEQ ID NO: 186 - protein, 322 aa, Artificial Sequence; identical to SEQ ID NO: 92
  Description of Artificial Sequence Synthetic polypeptide

SEQ ID NO: 187 - protein, 321 aa, Artificial Sequence; identical to SEQ ID NO: 93
  Description of Artificial Sequence Synthetic polypeptide

SEQ ID NO: 188 - protein, 323 aa, Artificial Sequence; identical to SEQ ID NO: 94
  Description of Artificial Sequence Synthetic polypeptide

SEQ ID NO: 189 - protein, 325 aa, Artificial Sequence; identical to SEQ ID NO: 95
  Description of Artificial Sequence Synthetic polypeptide

SEQ ID NO: 190 - protein, 323 aa, Artificial Sequence; identical to SEQ ID NO: 96
  Description of Artificial Sequence Synthetic polypeptide

SEQ ID NO: 191 - protein, 325 aa, Artificial Sequence; identical to SEQ ID NO: 97
  Description of Artificial Sequence Synthetic polypeptide

SEQ ID NO: 192 - protein, 327 aa, Artificial Sequence; identical to SEQ ID NO: 98
  Description of Artificial Sequence Synthetic polypeptide

SEQ ID NO: 193 - protein, 326 aa, Artificial Sequence; identical to SEQ ID NO: 99
  Description of Artificial Sequence Synthetic polypeptide

SEQ ID NO: 194 - protein, 328 aa, Artificial Sequence; identical to SEQ ID NO: 100
  Description of Artificial Sequence Synthetic polypeptide

SEQ ID NO: 195 - protein, 195 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 196 - protein, 210 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGGGGGSGGGGSGGGGSSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 197 - protein, 253 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 198 - protein, 268 aa, Artificial Sequence
  Description of Artificial Sequence Synthetic polypeptide
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKGGGGSGGGGSGGGGSSGMEPYACPVESCDRRFSQSGHLQRHIRIHTGQKPFQCRICMRNFSRSDALTRHIRTHTGEKPFACDICGRKFSDPGHLVRHTKIHLRQKQLVHHHHHH

SEQ ID NO: 199 - protein, 426 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 200 - protein, 439 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 201 - protein, 452 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 202 - protein, 481 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 203 - protein, 498 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDGGGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 204 - protein, 511 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGGGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVF

SEQ ID NO: 205 - protein, 517 aa, Artificial Sequence
  Description of Artificial Sequence Synthetic polypeptide
MKSGIYQIKNTLNNKVYVGSAKDFEKAWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGGGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 206 - protein, 517 aa, Artificial Sequence
  Description of Artificial Sequence Synthetic polypeptide
MKSGIYQIKNTLNNKVYVGSAKDFEKAWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNGGGGGSGSAYMSRRESINPWILTGFADAEGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

SEQ ID NO: 207 - protein, 509 aa, Artificial Sequence
  Description of Artificial Sequence Synthetic polypeptide
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKGSGSAYMSRRESINPWILTGFADAQGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVF

SEQ ID NO: 208 - protein, 1519 aa, Artificial Sequence; identical to SEQ ID NO: 118
  Description of Artificial Sequence Synthetic polypeptide

SEQ ID NO: 209 - protein, 1521 aa, Artificial Sequence; identical to SEQ ID NO: 119
  Description of Artificial Sequence Synthetic polypeptide

SEQ ID NO: 210 - protein, 1523 aa, Artificial Sequence; identical to SEQ ID NO: 120
  Description of Artificial Sequence Synthetic polypeptide

SEQ ID NO: 211 - protein, 1521 aa, Artificial Sequence; identical to SEQ ID NO: 121
  Description of Artificial Sequence Synthetic polypeptide

SEQ ID NO: 212 - protein, 1523 aa, Artificial Sequence; identical to SEQ ID NO: 122
  Description of Artificial Sequence Synthetic polypeptide

SEQ ID NO: 213 - protein, 1525 aa, Artificial Sequence; identical to SEQ ID NO: 123
  Description of Artificial Sequence Synthetic polypeptide

SEQ ID NO: 214 - protein, 1524 aa, Artificial Sequence; identical to SEQ ID NO: 124
  Description of Artificial Sequence Synthetic polypeptide

SEQ ID NO: 215 - protein, 1526 aa, Artificial Sequence; identical to SEQ ID NO: 125
  Description of Artificial Sequence Synthetic polypeptide

SEQ ID NO: 216 - protein, 486 aa, Artificial Sequence
  Description of Artificial Sequence Synthetic polypeptide
MSKSGIYQIKNTLNGKVYVGSAKDFEKRWKRHFKDLENGVHSSIKFQRSFNKHGNVFECSVLEEIPYEKDLIIERENFWIKELNSKINGYNIADASFGDVLSNHPLKEEIAKKRAETVKAKMLKLGPDGRKALYGKHGSKNGRWNPENHKFCKCGVRIPSSADTCGKCRKGGSGGSGSAYMSRRESINPWILTGFADAEGSFLLRIRNSNKRSVGYATELGFQICLHIKDKSILENIQSTWKVGVIANSGDNAVSLRVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSNSKLGVQVQLVFSITQHIKDKNLMNSLITYLGCGYIKEKNRSEFSWLDFVVTKFSDINDKIIPVFQENTLIGVKLEDFEDWCKVAKLIEEKKHLTESGLDEIKKIKLNMNKGRVFHHHHHH

(19) United States
(12) Patent Application Publication
(10) Pub. No.: US 2013/0210151 A1
(43) Pub. Date: Aug. 15, 2013

(54) ENDONUCLEASE FOR GENOME EDITING

(71) Applicant: University of Western Ontario, (US)

(72) Inventors: David Rohan Edgell, London (CA); Benjamin Peter Kleinstiver, London (CA)

(73) Assignee: University of Western Ontario, London (CA)

(21) Appl. No.: 13/671,452

(22) Filed: Nov. 7, 2012

Related U.S. Application Data
(60) Provisional application No. 61/628,810, filed on Nov. 7, 2011.
(60) Provisional application No. 61/701,545, filed on Sep. 14, 2012.

(51) Int. Cl. C12N 9/22

(52) U.S. Cl. CPC C12N 9/22
USPC 435/468; 435/199; 536/23.2; 536/23.1; 435/410; 435/320.1

(57) ABSTRACT

A chimeric endonuclease is provided comprising the GIY-YIG nuclease domain which is linked to a DNA-targeting domain by a linking domain. The endonuclease is useful in gene editing.

### CROSS REFERENCE TO RELATED APPLICATIONS

The present application claims benefit of U.S. Provisional Application Nos. 61/628,810 filed Nov. 7, 2011, and 61/701,545 filed Sep. 14, 2012, the entire contests of both of which are hereby specifically incorporated by reference in their entireties.

### FIELD OF THE INVENTION

The present application relates generally to endonucleases useful for gene editing.

### BACKGROUND OF THE INVENTION

Precise genome editing is enhanced by the introduction of a double-strand break (DSB) at defined positions, and two distinct site-specific DNA endonuclease architectures have been developed towards this goal. One of these architectures relies on reprogramming the DNA-binding specificity of naturally occurring LAGLIDADG (SEQ ID NO:1) homing endonucleases (LHEs) to target desired sequences. The other architecture utilizes the reprogrammable DNA-binding specificity of zinc-finger proteins or the DNA-binding domains of transcription activator-like effectors (TAL-effectors) that are fused to the non-specific nuclease domain of the type IIS restriction enzyme FokI to create chimeric zinc-finger nucleases (ZFNs) or TAL-effector nucleases (TALENs). Regardless of the architecture, the underlying biology of the component proteins imposes design challenges and the relative merits of the LHE and the ZFN/TALEN architectures are the subject of much debate in the literature. One notable constraint imposed by the FokI nuclease domain is the requirement to function as a dimer to efficiently cleave DNA. For any given DNA target this necessitates the design of two distinct ZFNs (or two TALENs), such that each zinc finger or TAL-effector domain is oriented to promote FokI dimerization and DNA cleavage. Off-target DSBs have been observed with ZFNs, likely promoted by binding at degenerate site and by DNA-bound ZFNs recruiting ZFNs in solution to promote DNA hydrolysis. Many engineering strategies have been employed with varying degrees of success to reduce off-target effects, including creating sets of complementary heterodimeric nuclease domains, addition of zinc-finger modules, optimization of the FokI-zinc finger linker, and in vitro and in vivo selections to increase zinc-finger binding specificity.

Expanding the repertoire of DNA nuclease domains with distinctive properties is necessary to facilitate the development of new genome editing reagents. Indeed, a number of recent studies have explored the potential of alternative dimeric sequence-specific nuclease domains for genome editing applications. These dimeric nuclease domains, however, still require the design of two nuclease fusions for precise targeting. The GIY-YIG nuclease domain is associated with a variety of proteins with diverse cellular functions. The small (˜100 aa) globular GIY-YIG domain is characterised by a structurally conserved central three-stranded antiparallel β sheet, with catalytic residues positioned to utilize a single metal ion to promote DNA hydrolysis. Intriguingly, the GIY-YIG homing endonucleases, typified by the isoschizomers I-TevI (a double-strand DNA endonuclease encoded by the mobile td intron of phage T4), I-BmoI and I-TulaI bind DNA as monomers. It is unknown, however, if GIY-YIG homing endonucleases function as monomers in all steps of the reaction, as it is possible that dimerization between GIY-YIG nuclease domains is necessary for efficient DNA hydrolysis, as is the case with FokI. Notably, GIY-YIG homing endonucleases require a specific DNA sequence to generate a DSB. For I-TevI, the bottom (↑) and top (↓) strand nicking sites lie within a 5′-CN↑N↓G-3′ motif (referred to as CNNNG or CXXXG), with the critical G optimally positioned ˜28 bp from the where the H-T-H module of the I-TevI DNA-binding domain interacts with substrate.

It would be desirable to develop novel endonucleases for use in genome editing that overcome one or more disadvantages of existing endonucleases.

### SUMMARY OF THE INVENTION

The present invention provides chimeric endonucleases and methods of making and using such chimeric endonucleases. In one embodiment of the invention, the present invention provides a chimeric endonuclease comprising at least a nuclease domain and a DNA-targeting domain. Typically, the nuclease domain has the ability to cleave double-stranded DNA, typically at a specific DNA sequence. In some embodiments, the nuclease is capable of cleaving double-stranded DNA as a monomer. The nuclease domain may be derived from a homing endonuclease. Suitable examples of homing endonucleases include, but are not limited to, homing endonucleases of the LAGLIDADG, HNH, His-Cys box, and GIY-YIG families. In one embodiment of the invention, a chimeric endonuclease of the invention comprises a nuclease domain derived from a homing endonuclease of the GIY-YIG family. Suitable examples of homing endonucleases of the GIY-YIG family include, but are not limited to, I-TevI and I-BmoI. In some embodiments, a chimeric endonuclease of the invention comprises the nuclease domain of I-TevI. Chimeric endonucleases of the invention may be provided as part of a composition, for example, a pharmaceutical composition. The present invention also provides cells, cell lines and transgenic organisms (e.g., plants, fungi, animals) composing one or more chimeric endonucleases of the invention. Suitable cells include, but are not limited to, mammalian cells (e.g., mouse cells, human cells, rat cells, etc.) which may be stem cells, avian cells, plant cells, bacterial cell, fungal cells (e.g., yeast cells), and any other type of cell known to those skilled in the art.

Any specific DNA-binding domain known to those skilled in the art may be used as a DNA-targeting domain in the practice of the present invention. Examples include, but are not limited to, the DNA-binding domains of TAL-effector proteins (which will be referred to herein as TAL domains), such as PthXoI and AvrBs3 (from Xanthamonas campestris); zinc finger domains, e.g. ryA zinc finger binding domain and ryB zinc finger binding domain, and other distinct DNA-binding domains, such as the binding domain in LADLIDADG homing endonucleases, for example I-OnuI. In some embodiments, the entire LAGLIDADG homing endonuclease, not just the binding domain, may be used as a DNA-targeting domain in the practice of the present invention. In some embodiments, the nuclease activity of the LADLIDADG endonuclease may be disrupted, for example, with a point mutation, such last it acts as a DNA-binding platform only.

In some embodiments, a chimeric endonuclease of the invention may comprise one or more additional domains. Examples of additional domains include, but are not limited to, linking domains and functional domains. Typically, linking domains may be disposed between two functional domains, for example, between a nuclease domain and a DNA-targeting domain. Other functional domains include domains comprising nuclear localization signals, transcription activating domains, dimerization domains, and other functional domains known to those skilled in the art.

The present invention also provides nucleic acid molecules encoding the chimeric endonucleases of the invention. Such molecules may be DNA or RNA. Typically, DNA molecules will comprise one or more promoter regions operably linked to a nucleic acid sequence encoding all or a portion of a chimeric endonuclease of the invention. Nucleic acid molecules of the invention may be provided as part of a larger nucleic acid molecule, for example, an expression vector. Suitable expression vectors include, but are not limited to, plasmid vectors, viral vectors, and retroviral vectors. Nucleic acid molecules of the invention may be provided as part of a composition, for example, a pharmaceutical composition. The present invention also provides cells, cell lines and transgenic organisms (e.g., plants, fungi, animals) comprising one or more nucleic acid molecules of the invention. Suitable cells include, but are not limited to, mammalian cells (e.g., mouse cells, human cells, rat cells, etc.) which may be stem cells, avian cells, plant cells, insect cells, bacterial cells, fungal cells (e.g., yeast cells), and any other type of cell known to those skilled in the art.

In a further embodiment of the invention, a method of cleaving a target nucleic acid is provided comprising the step of exposing target nucleic acid to a chimeric endonuclease as defined above, wherein the DNA targeting domain of the endonuclease binds to the target nucleic acid and the nuclease domain cleaves the target nucleic acid. In some embodiments, the target nucleic acid may be a gene of interest in a cell. Thus, methods of the invention may be used in genomic editing applications. Typically a method of this type will comprise introducing, into the cell, one or more one chimeric endonucleases of the invention that bind to a target nucleic acid sequence in the gene (or nucleic acid molecules encoding such chimeric endonuclease under conditions resulting in expression of the chimeric endonucleases), wherein the DNA-targeting domain of the endonuclease binds to the target nucleic acid sequence and the nuclease domain cleaves the target nucleic acid. In some embodiments, cleavage of the gene results in disrupting the function of the gene as repair of the double-stranded break introduced by the chimeric endonuclease of the invention may result in one or more insertions and or deletions of nucleotides at the site of the break.

In another embodiment, the present invention provides a method for introducing an exogenous nucleotide sequence into the genome of a cell. Such methods typically comprise, introducing, into the cell, one or more chimeric endonucleases of the invention (or nucleic acid molecules encoding such chimeric endonucleases under conditions resulting in expression of the chimeric endonucleases), wherein the DNA-targeting domain of the endonuclease binds to the target nucleic acid and the nuclease domain cleaves the target nucleic acid, and contacting the cell with an exogenous polynucleotide; under conditions such that the exogenous polynucleotide is integrated into the genome by homologous recombination. In some embodiments, the exogenous polynucleotide may comprise a nucleic acid sequence that is capable of interacting with a protein. Suitable examples of such sequences include, but are not limited to, recognition sites (e.g., endonuclease recognition sites, recombinase recognition sites), promoter sequences, and protein binding sites.

In some embodiments, the present invention provides a chimeric endonuclease. Such a chimeric endonuclease typically comprises a nuclease domain and a DNA-targeting domain. In some embodiments, the chimeric endonuclease is capable of cleaving double-stranded DNA as a monomer. In some embodiments, the nuclease domain is a site-specific nuclease domain, which may be from a homing endonuclease. A suitable example of a homing endonuclease is a GIY-YIG homing endonuclease, for example I-TevI. A chimeric endonuclease of the invention may further comprise a linking domain. In some embodiments, the DNA-targeting domain is a TAL domain. In one embodiment, the chimeric endonuclease comprises a I-TevI nuclease domain and a TAL DNA-targeting domain. In some embodiments, I-TevI nuclease is N-terminal to the TAL domain. The present invention also provides nucleic acid molecules encoding chimeric endonucleases as described above.

In some embodiments, the present invention provides a method of inactivating a gene. Such methods typically comprise introducing into a cell comprising the gene a nucleic acid molecule encoding a chimeric endonuclease as described above under conditions causing the expression of the chimeric endonuclease. Typically the chimeric endonuclease comprises a DNA-targeting domain that binds the gene and cleaves it. In some embodiments, the expression of the chimeric endonuclease is transient. In some embodiments, the cell is a plant cell. In some embodiments, the nucleic acid molecule is an mRNA.

In some embodiments, the present invention provides a method of altering a gene in a cell. Such methods typically comprise introducing a first nucleic acid molecule encoding a chimeric endonuclease as described above into a cell comprising the gene under conditions causing the expression of the chimeric endonuclease and cleavage of the gene. Such methods may further comprise introducing a second nucleic acid molecule into the cell. Typically, the second nucleic acid molecule comprises a region having a nucleotide sequence that has a high degree of sequence identity to all or a portion of the gene in the region of the cleavage site. The second nucleic acid molecule is introduced under conditions causing homologous recombination to occur between the second nucleic acid molecule and the gene. In some embodiments, the region of high sequence identity comprises a sequence that is highly identical to all or a portion of the sequence of the gene. In some embodiments, the region of high sequence identity of the second nucleic acid molecule is not 100% identical to the corresponding region of the gene. Instead the region comprises an altered sequence when compared to the gene of interest. Typically, the region may comprise one or more mutations that will result in changes to one or more amino acids in a protein encoded by the gene. In some embodiments, the chimeric endonuclease is transiently expressed in the cell. In some embodiments, the first nucleic acid molecule is mRNA. In some embodiments, the second nucleic acid molecule is a linear DNA molecule. In some embodiments, the cell is a plant cell.

The present invention provides method for deleting all or a portion of a gene in a cell. Such methods typically comprise introducing a first nucleic acid molecule encoding a chimeric endonuclease as described above into a cell comprising the gene under conditions causing expression of the chimeric endonuclease and cleavage of the gene. A second nucleic acid molecule comprising a region having a nucleotide sequence that has a high degree of sequence identity to the gene in the region of the cleavage site is introduced into the cell under conditions causing homologous recombination to occur between the second nucleic acid molecule and the gene. Typically, the region of high sequence identity lacks the sequence of the gene adjacent to the cleavage site. In some embodiments, the region of high sequence identity comprises a sequence that is highly identical to all or a portion of the sequence of the gene. In some embodiments, the region of high sequence identity of the second nucleic acid molecule is not 100% identical to the corresponding region of the gene. Instead the region comprises an altered sequence when compared to the gene of interest. In some embodiments, the region comprises one or more mutations that will result in changes to one or more amino acids in a protein encoded by the gene. In some embodiments, the chimeric endonuclease is transiently expressed in the cell. In some embodiments, the first nucleic acid molecule is mRNA. In some embodiments, the second nucleic acid molecule is a linear DNA molecule. In some embodiments, the cell is a plant cell.

The present invention provides a method tor making a cell having an altered genome. Such methods typically comprise introducing into the cell a first nucleic acid molecule encoding a chimeric endonuclease as described above under conditions causing expression of the chimeric endonuclease and cleavage of the gene. In some embodiments, the altered genome comprises an inactivated gene. Methods of making a cell having an altered genome may also comprise introducing into the cell a second nucleic acid molecule comprising a region having a nucleotide sequence that has a high degree of sequence identity to the gene in the region of the cleavage site. The second nucleic acid molecule is introduced into the cell under conditions causing homologous recombination between the gene and the second nucleic acid, wherein the region of high sequence identity comprises an altered sequence when compared to the gene. In some embodiments, the region of high sequence identity comprises a sequence that is highly identical to all or a portion of the sequence of the gene. In some embodiments, the region comprises one or more mutations that will result in changes to one or more amino acids in a protein encoded by the gene. In some embodiments, the nucleotide sequence of the region lacks the sequence of the gene adjacent to the cleavage site. In some embodiments, the chimeric endonuclease is transiently expressed in the cell. In some embodiments, the first nucleic acid molecule is mRNA. In some embodiments, the second nucleic acid molecule is a linear DNA molecule. In some embodiments, the cell is a plant cell.

The present invention provides a nucleic acid substrate for the chimeric endonuclease as described above. Such a substrate will typically comprise a cleavage motif of the nuclease domain, a spacer that correlates with the linking domain and a binding site for the DNA-targeting domain. The present invention also provides cells, for example plant cells, incorporating the substrate.

The present invention provides kits comprising nucleic acid molecules encoding the chimeric endonucleases described above and a substrate for the chimeric endonuclease. In another embodiment, the invention provides kits comprising the chimeric endonucleases of the invention. Kits of the invention can be used for genomic editing using the methods described above.

These and other aspects of the invention will become apparent from the detailed description by reference to the following figures.

### BRIEF DESCRIPTION OF THE FIGURES

FIG. 1 illustrates that I-BmoI functions as a monomer. FIG. 1A provides graphs of progress curves of initial reaction velocity for eight I-BmoI concentrations with fixed amount (10 nM) of pBmoIHS target site plasmid (left) and plot of initial velocity versus I-BmoI protein concentration (right). FIG. 1B provides graphs showing results of time course assays showing cleavage of 1- or 2-site target plasmids by I-BmoI;

FIG. 2 schematically illustrates the design and functionality of chimeric GIY-YIG endonucleases of the invention. FIG. 2a provides a schematic modeling of a Tev-zinc finger fusion with DNA substrate using structures of the I-TevI catalytic domain (PDB 1MK0), the I-TevI DNA-binding domain co-crystal (PDB 1I3J), and the Zif268 co-crystal (PDB 1AAY). FIG. 2b (upper) provides a schematic of a chimeric I-TevI endonuclease-ryA construct showing the fusion point as the last I-TevI amino acid, with an optional 2× Glycine or 4× Glycine linker and 6×His tag at the C-terminal end, and (lower) a Tev-ryA substrate including 33-nts of the top strand of the I-TevI td homing site substrate (TZ1.33), fused to the 5′ end of the ryA-binding site. The substrate is numbered from the first base of the td homing site sequence (note that is numbering scheme is reverse of that used for the native td homing site). The different substrates tested differ by one or two T residues inserted at the junction of the td/ryA sites. FIG. 2c (upper) provides a schematic of a chimeric I-BmoI endonuclease-ryA construct showing the fusion point as the last I-BmoI amino acid, with an optional 2× Glycine or 4× Glycine linker and 6×His tag at the C-terminal end, and (lower) a I-BmoI-ryA substrate including 33-nts of the top strand of the I-BmoI homing site substrate (BZ1.33), fused to the 5′ end of the ryA-binding site. FIG. 2d provides a schematic representation of the two plasmids used in the genetic selection system, where the fusion protein is expressed from pExp and the hybrid targets sites are cloned onto the pTox plasmid harboring the ccdB gyrase toxin;

FIG. 3 shows chimeric GIY-YIG endonuclease target specificity. FIG. 3a is an SDS-PAGE that shows purification of TevN201-zinc finger endonuclease (ZFE). FIG. 3b is an SDS-PAGE that shows purification of a BmoN221-ZFE. Lanes are marked as follows: M, marker with molecular weights in kDa indicated on the left; UN, uninduced culture; IND, induced culture; C, crude lysate; FT, flow-through from metal-affinity column; W, wash; E, elution. FIG. 3c is a sequencing gel that shows mapping of TevN201-ZFE cleavage sites on the TZ1.33 substrate, with top and bottom cleavage sites indicated below on the Tev-ryA substrate by open and closed triangles, respectively. FIG. 3d is a sequencing gel that shows mapping of BmoN22I-ZFE cleavage sites on the BZ1.33 substrate, with top and bottom cleavage sites indicated below on the Bmo-ryA substrate. FIG. 3e (left) shows the sequences of the wild-type TZ1.33, the TZ1.33 G5A, and TZ1.33 C1A/G5A mutant substrates and (right) is a bar graph that shows the EC_{0.5max} determinations for each substrate, with EC_{0.5max} values in nM with standard deviations fern three experimental trials;

FIG. 4A provides the amino acid sequences of chimeric GIY-YIG I-TevI endonucleases of the invention. FIG. 4B provides the amino acid sequences of chimeric I-BmoI endonucleases of the invention

FIG. 5 illustrates that TevN201-ZFE functions as a monomer. FIG. 5a (left) is a graph of initial reaction progress for seven TevN201-ZFE concentrations expressed as percent linear product. Protein concentrations from highest to lowest are 47 nM, 32.5 nM, 23 nM, 11 nM, 6 nM, 3 nM, and 0.7 nM. FIG. 5a (right) is a graph of initial reaction velocity (nM s⁻¹) versus TevN201-ZFE concentration (nM). FIG. 5b provides graphs of the results of cleavage assays with 90 nM TevN201-ZFE and 10 nM one-site pTZ1.31 plasmid (left), or two-site pTZ1.31 plasmids with the same orientation of sites (center) and two-site pTZ1.31 plasmids with the opposite orientation of sites (right);

FIG. 6 provides a schematic comparison of GIY-YIG ZFEs and ZFNs. (upper) The GIY-YIG nuclease fusion is to the ryA zinc finger, and (lower) the two ZFNs are fusions of the FokI nuclease domain to ryA and ryB zinc fingers. The central portion of the GIY-YIG ZFE substrate is shown as random sequence (N).

FIG. 7 shows various GIY-YIG TAL domain chimeric endonuclease constructs of the invention. FIG. 7A (upper) is a schematic of the chimeric endonuclease I-TevI/PthXol fusion proteins including amino acid sequences of I-TevI/PthXol fusion proteins, (lower) shows the sequences of various hybrid I-TevI/PthXol substrates. FIG. 7B provides the amino acid sequence of various I-TevI/PthXol chimeric endonucleases of the invention. FIG. 7C provides the sequences of various I-TevI/PthXol hybrid target sites. FIG. 7D shows the amino acid sequences of various I-BmoI/PthXol chimeric endonucleases of the Invention. FIG. 7E shows the sequences of various I-BmoI/PthXol target sites.

FIG. 8 is photograph of an ethidium bromide gel showing the double-stranded cleavage of various sized substrates;

FIG. 9 (upper) is a schematic of the assay used to individually demonstrate cleavage of top and bottom strands (lower) is a gel showing the results of the assay with variously sized substrates;

FIG. 10A is a schematic of an in vitro endonuclease selection protocol. FIG. 10B is a graph illustrating the frequency of each nucleotide at various positions in a substrate space as determined by the assay of FIG. 10 A. A positive value means an increase in nucleotide frequency, while a negative value means a decrease in nucleotide frequency. Note that position 15 can be mutated without effect on activity. FIG. 10C is a schematic showing a correlation of the sequence of the DNA spacer binding motif with the I-TevI binding domain. The figure shows a correlation between the preferred DNA bases in the DNA spacer region of the substrate with conserved DNA bases of the native I-TevI target site in thymidylate synthase genes. Homing endonucleases, such as I-TevI, target genes that encode for conserved proteins. Doing so maximizes their opportunity to spread between related genomes. Further, the homing endonucleases target DNA sequence that corresponds to conserved amino acids of the target gene—again, by using these DNA sequences as recognition determinants it maximizes potential to spread. This figure was using this correlation as a justification for why those positions in the DNA spacer are important;

FIG. 11 graphically illustrates the frequency of the I-TevI cleavage motif in human cDNAs;

FIG. 12A provides the sequences of the target substrates isolated from a bacterial two plasmid genetic selection assay, and 12B is a bar graph showing percent survival based on substrate spacers as determined by the assay;

FIG. 13 graphically illustrates the results of a yeast assay for a TevN169 endonuclease using substrates shown in FIG. 12. Substrate TO20 has the following sequence 5-CAACGCTCAGTAGATGTTTTGGTCCACATATTTAACCTTTTG-3 (SEQ ID NO:2), Substrate Zif268 has the following sequence 5-GCGTGGGCG-3 (SEQ ID NO:3);

FIG. 14 graphically illustrates the results of a yeast assay for a TulaK169 endonuclease using substrates shown in FIG. 12(A);

FIG. 15A provides the amino acid sequence of endonuclease I-BmoI. FIG. 15B provides the amino acid sequence of endonuclease I-TevI. FIG. 15C provides the amino acid sequence of endonuclease I-TulaI. FIG. 15D provides an amino acid alignment of the linker regions of I-TulaI, I-TevI, and I-BmoI;

FIG. 16A provides the amino acid sequences of DNA binding proteins, PthXol, AvrBs3, ryA, ryB and I-OnuI. FIG. 16B provides the sequences of the binding sites of each;

FIG. 17A provides the amino acid sequences of various I-TevI-zinc finger chimeric endonucleases. FIG. 17B provides the amino acid sequences of various I-BmoI-zinc finger chimeric endonucleases;

FIG. 18 provides the amino acid sequences of I-TevI-I-OnuI chimeric endonucleases;

FIG. 19 provides the amino acid sequences of I-TevI-TAL chimeric endonucleases; and

FIG. 20 provides the amino acid sequence of an I-TulaI-ONU chimeric endonuclease.

FIG. 21 provides a sequence alignment of two TAL-effector proteins Avrb6 from Xanthomonas citri subsp. Malvacearum GenBank accession number AAB00675.1 and PthN from Xanthomonas campestris GenBank accession number AAB69865.1

### DETAILED DESCRIPTION OF THE INVENTION

The present invention provides novel chimeric endonucleases that can be engineered to cleave virtually any nucleic acid molecule at a desired site. This is accomplished by selecting the desired binding and cleaving domains and using recombinant DNA techniques to construct a fusion protein comprising the selected domains. Thus, chimeric endonucleases invention are capable of creating double-stranded breaks in DNA molecule, for example, in the genome of an organism. Double-stranded breaks thus created may be used, for example, to induce targeted mutagenesis, induce targeted deletions of cellular DNA sequences, and facilitate targeted recombination at a predetermined chromosomal locus. See, for example, United States Patent Publications 20030232410; 20050208489; 20315002615; 20050064474; 20060188987; 20060063231; 20070218528; 20070134796; 20080015164 and International Publication Nos. WO 07/014,275 and WO 2007/139982, the disclosures of which are specifically incorporated herein by reference in their entireties.

As an example, a novel chimeric endonuclease has now been developed comprising a GIY-YIG nuclease domain which is linked to a DNA-targeting domain by a linking domain. Unlike chimeric endonuclease of the prior art, for example, TALENs comprising the FokI nuclease domain, chimeric endonucleases of the present invention are capable of cleaving DNA as monomers. This allows greater flexibility in construction and ease in use as compared to the chimeric endonucleases of the prior art. Chimeric endonucleases of the invention will be particularly useful for in vivo applications as they do not require dimerization in situ to be effective.

Any site specific nuclease that is functional as a monomer can be used as the source of the nuclease domain for use in the present invention. In one embodiment, the nuclease domain is derived from a homing endonuclease, for example, a homing endonuclease of the GIY-YIG family of homing endonucleases. Other examples of site specific nucleases that cleave double-stranded DNA as monomers include, but are not limited to, MspI, HinPlI, MvaI and BcnI.

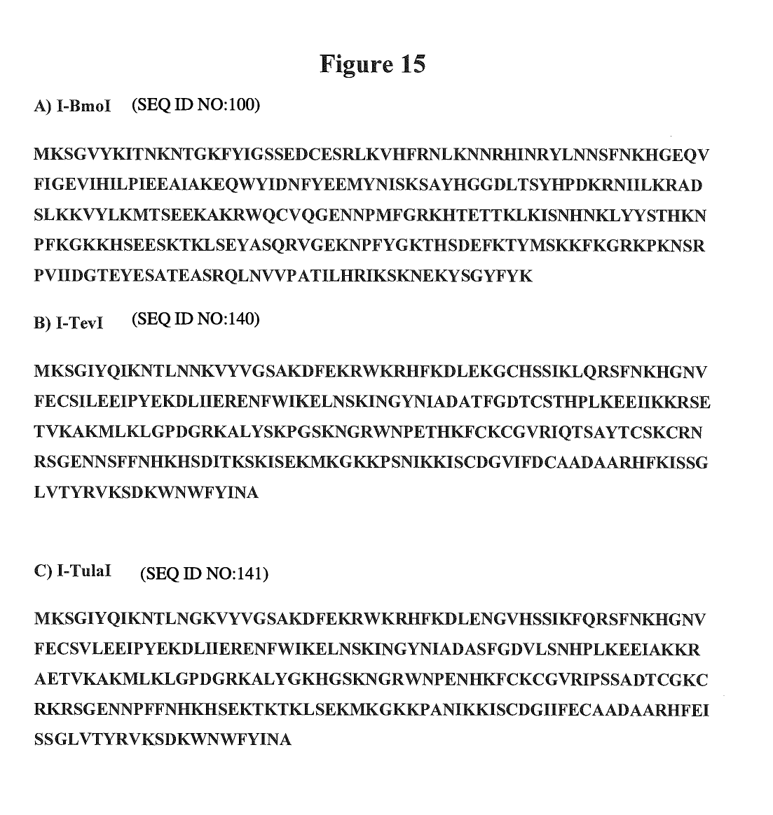

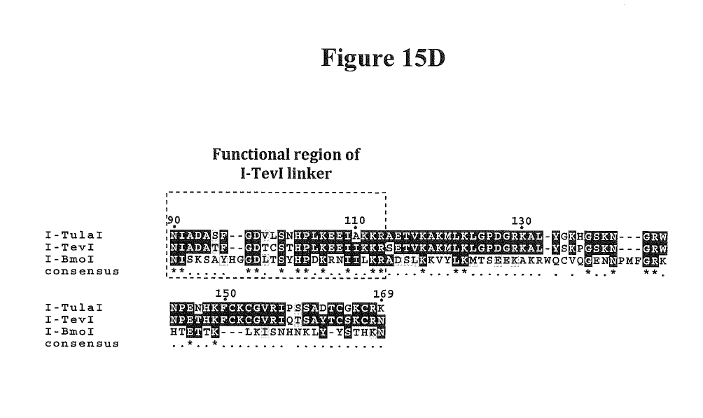

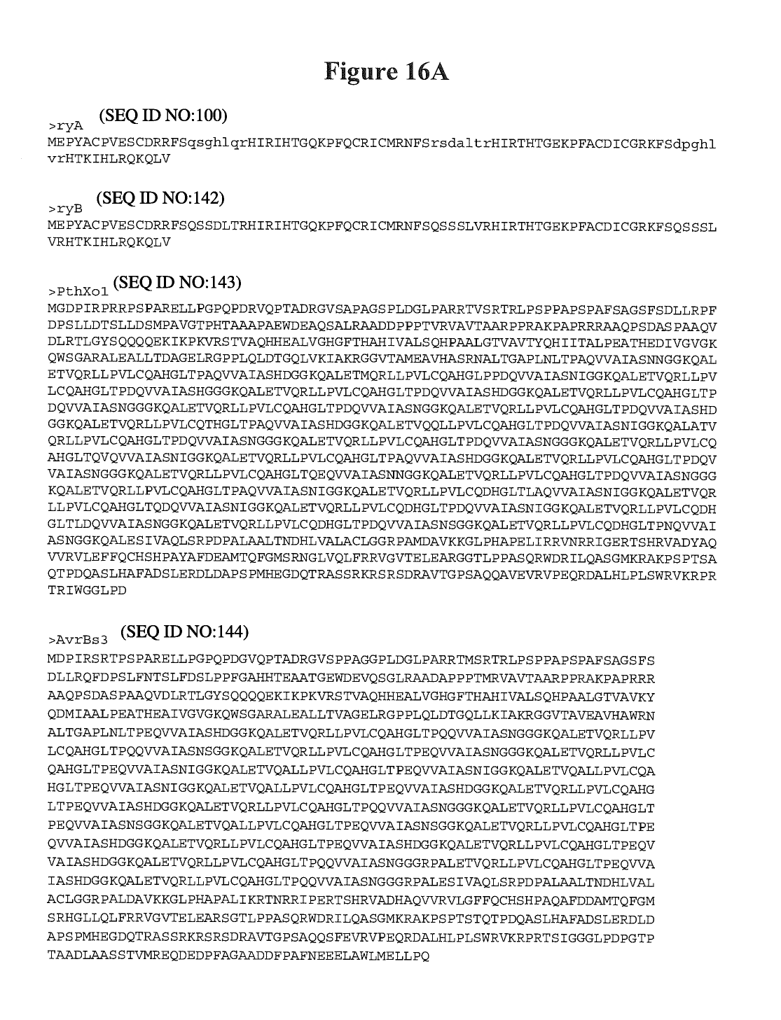

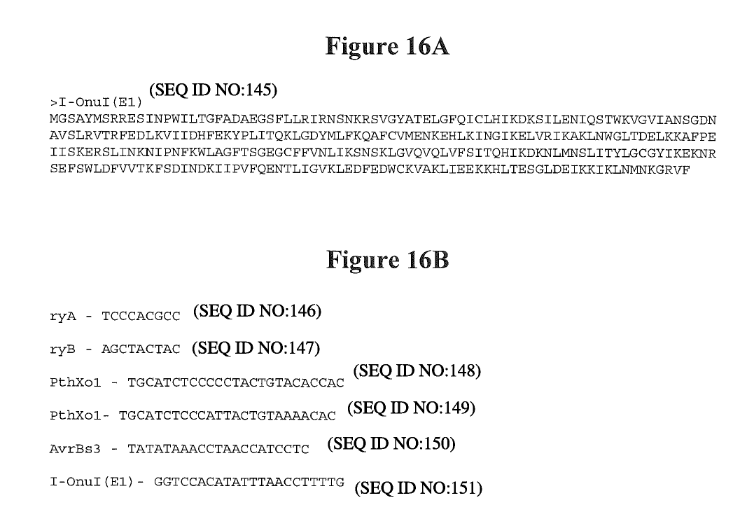

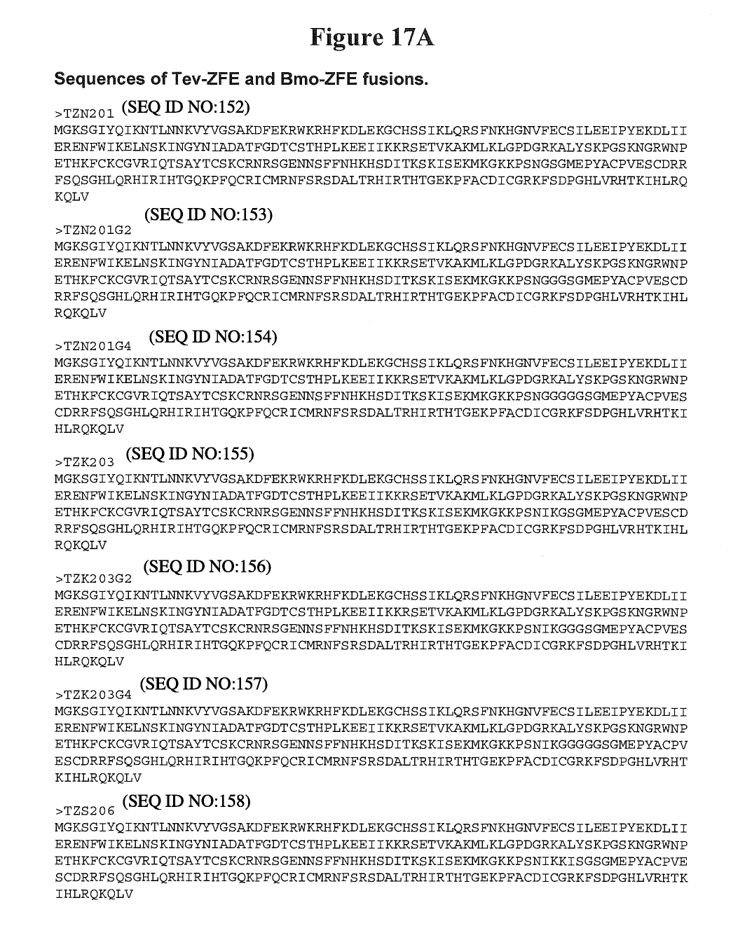

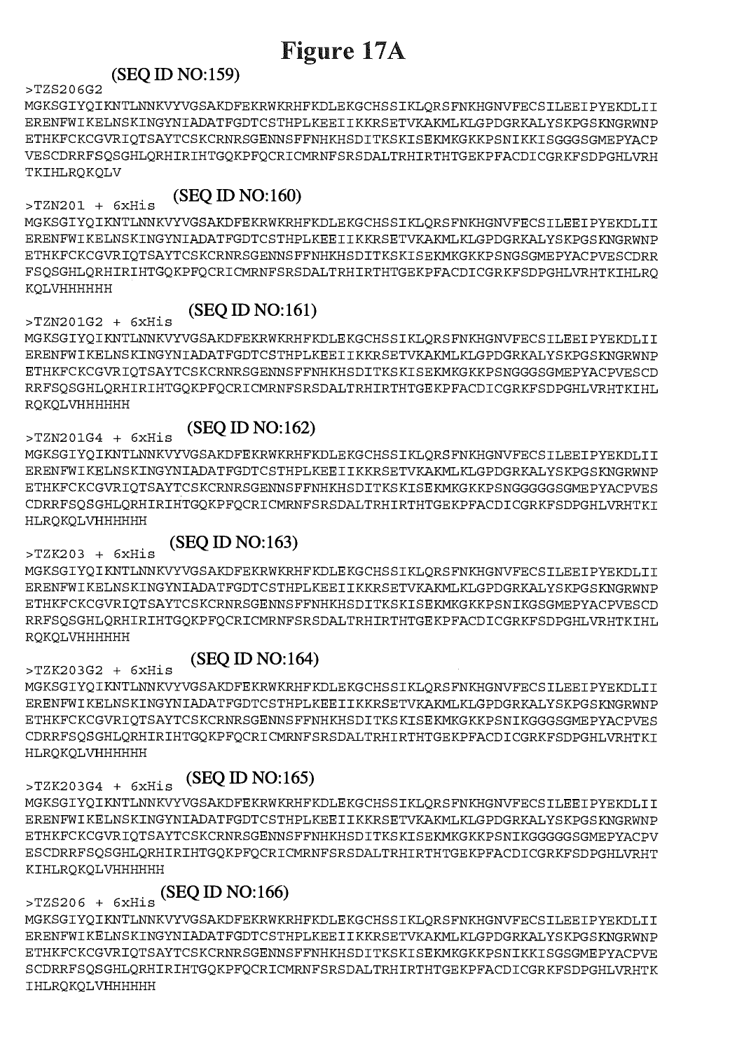

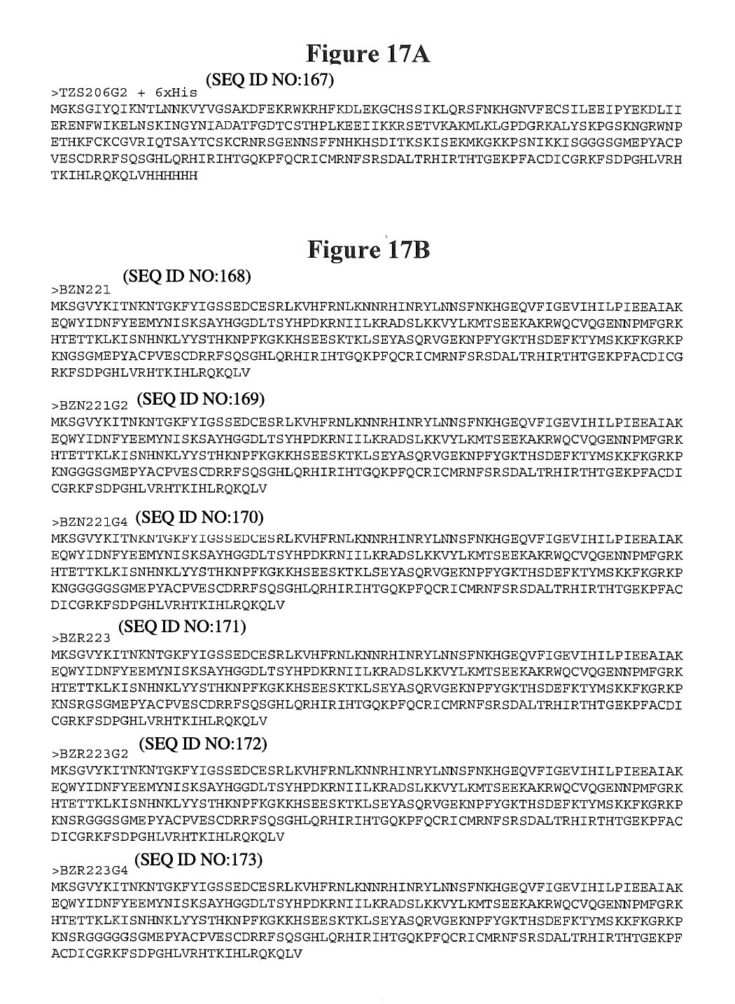

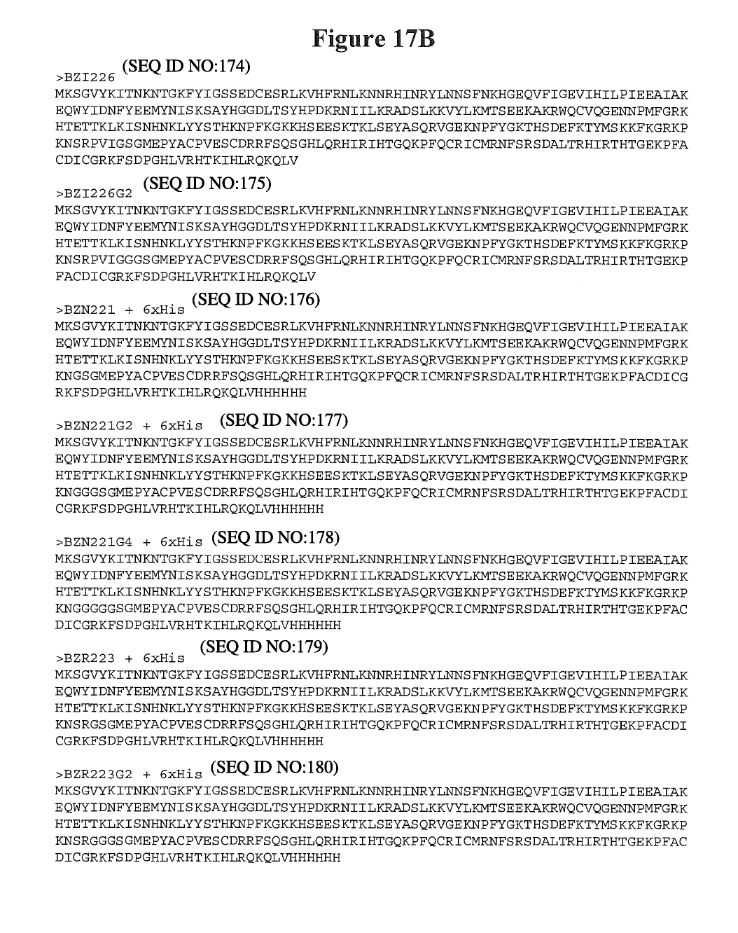

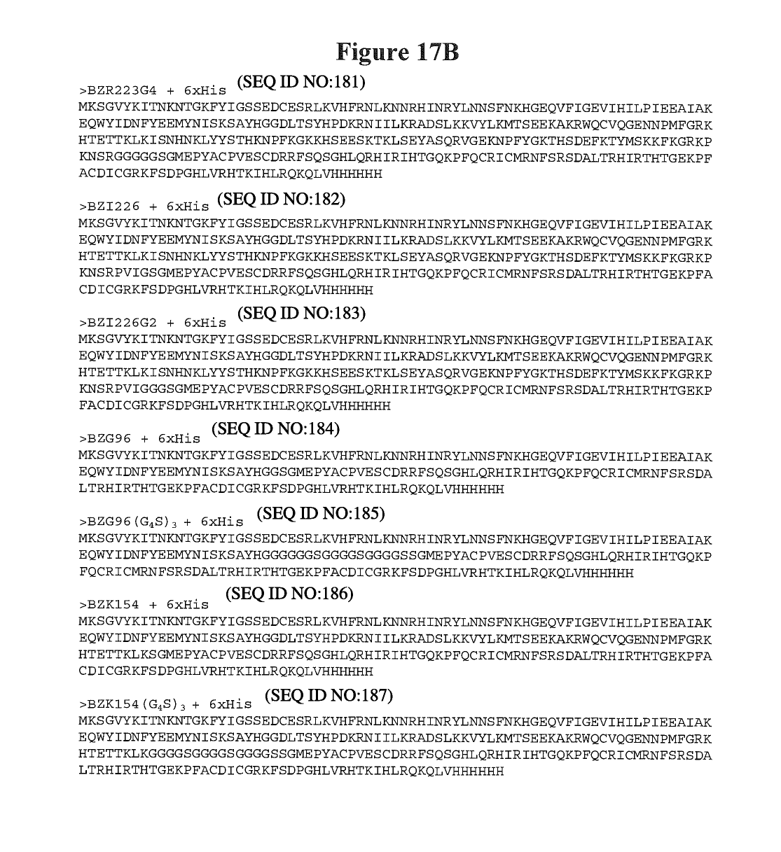

The present chimeric GIY-YIG endonuclease may comprise a GIY-YIG nuclease domain from any GIY-YIG homing endonuclease. As used herein, the GIY-YIG nuclease domain is an α/β structure comprising at least about 90-100 amino acids, the amino acid sequence -GIY- spaced from the amino acid sequence -YIG- by 10-11 amino acids which forms part of a three-stranded antiparallel β-sheet. Residues that may be important for nuclease activity include a glycine residue within the GIY-YIG motif, an arginine residue about 8-10 residues downstream, of the -GIY- sequence (e.g. arginine 27 of I-TevI), a metal-binding glutamic acid residue such as the glutamic acid at position 75 of I-TevI and a conserved asparagine about 14-16 residues upstream of the metal-binding glutamic acid residue (asparagine 90 of I-TevI) in the nuclease domain. Examples of suitable GIY-YIG nuclease domains include, but are not limited to, the nuclease portion of I-BmoI (for example, residues 1-92), the full-length amino acid sequence of which is illustrated in FIG. 15A, I-TevI (for example, at least residues 1-114), the full-length sequence of which is illustrated in FIG. 15B, and I-TulaI (for example, residues 1-114), the full-length sequence of which is illustrated in FIG. 15C.

As one of skill in the art will appreciate, functionally equivalent variant GIY-YIG nuclease domains may also be utilized within the present chimeric endonuclease. The term “functionally equivalent” refers to variant nuclease domains which vary from a wild-type or endogenous sequence but which retain nuclease function, even though it may be to a lesser degree. Accordingly, variant GIY-YIG nuclease domains may include one or more amino acid substitutions, deletions or insertions at positions which do not eliminate nuclease activity. Variant nuclease domains may comprise at least about 50% sequence similarity with a native nuclease sequence, at least about 60-70%, or at least about 80%-90% or greater sequence similarity with a native nuclease sequence, to retain sufficient nuclease activity. Examples of variant GIY-YIG nuclease domains include N- or C-terminal truncated GIY-YIG nuclease domains, for example, N-terminal truncations of up to about 20 amino acid residues and C-terminal truncations of up to about 15 amino acid residues, and one or more amino acid substitutions, insertions or deletions which do not adversely affect nuclease activity, for example within the N-terminus up to about the amino acid at position 20 or within the C-terminus from about the amino acid at position 75, and amino acid substitutions within the 10-11 amino acid spacer between -GIY- and -YIG-. In this regard, suitable amino acid substitutions include conservative amino acid substitutions, for example, substitution of an amino acid with a hydrophobic side chain with a like amino acid, e.g. alanine, valine, leucine, isoleucine, phenylalanine and tyrosine; substitution of an amino acid with an uncharged polar sidechain with a like amino acid, e.g. serine, threonine, asparagine and glutamine; substitution of an amino acid having a positively charged sidechain with a like amino acid, e.g. arginine, histidine and lysine; or substitution of an amino acid having a negatively charged sidechain with a like amino acid, e.g. aspartic and glutamic acid. Variant GIY-YIG nuclease domains may also include one or more modified amino acids, for example, amino acids including modified sidechain entities which do not adversely affect nuclease activity.

The GIY-YIG nuclease domain may be linked to a DNA-targeting domain via a linking domain. The linking domain will generally be a polypeptide of a length sufficient to permit the nuclease domain to retain nuclease function when linked to the DNA-targeting domain, and sufficient to permit the DNA-binding domain to bind the endonuclease to a target substrate. The linking domain may be from 1 amino acid residue to about 100 amino acid residues, from about 1 amino acid residue to about 90 amino acid residues, from about 1 amino acid residue to about 60 amino acid residues, from about 1 amino acid residue to about 70, from about 1 to about 60 amino acid residues, from about 1 to about 50 amino acid residues, from about 1 to about 40 amino acid residues, from about 1 to about 30 amino acid residues, or from about 1 amino acid residue to about 25 amino acid residues. The linking domain may be 1, 2, 3, 4, 5, 6, 7, 8, 9, 10, 11, 12, 13, 14, 15, 16, 17, 18, 19, 20, 21, 22, 23, 24, or 25 ammo acid residues in length.

The length of the linker domain may be adjusted depending on the distance between the binding and cleavage sites on a target nucleic acid molecule. By including an appropriately sized linker, chimeric endonucleases of the invention can cleave nucleic acid molecules where the binding and cleavage sites are separated by varying numbers of basepairs.

The linking domain may be a random sequence, for example, may be one or more glycine residues. The linking domain may be a simple repeat of amino acids, for example, GS, which may be repeated multiple times. As used herein, such a repeat will be indicated by placing the amino acids in parenthesis and using a subscript to indicate the number of times repeated. Thus (GS)₄ indicates a linking domain of four repeats of the amino acids glycine and serine. Similarly, (G₄S)₃ indicates three repeats of the sequence G-G-G-G-S. In some embodiments, the linker domain may comprise one or more glycine residues in addition to one or more amino acid residues. The linking domain may be from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, or may be 100% glycine. The linking domain may be flexible or may comprise one or more regions of secondary structure that impart rigidity, for example, alpha helix forming sequences. The linking domain may be the endogenous linker associated with the GIY-YIG nuclease, e.g. the linker region of I-TevI including amino acid residues 93-169, the linker region of I-Bmo-I including amino acids 90-149, or the linker region of I-TulaI including amino acids 93-169. Alternatively, the linking domain may be unrelated to the nuclease domain, i.e. the I-TevI linker or portion thereof may be utilized with the I-BmoI or I-TulaI nuclease regions, or the I-BmoI or I-TulaI linker or portion thereof may be used with the I-TevI nuclease domain. Various lengths of the nuclease-linker portion of an endonuclease may be utilized, such as the I-TevI nuclease domain and its linker region from about amino acid residue 1 to about amino acid residue 114, from about amino acid residue 1 to about amino acid residue 128, from about amino acid residue 1 to about amino acid residue 141, from about amino acid residue 1 to about amino acid residue 169, from about amino acid residue 1 to about amino acid residue 170, from about amino acid residue 1 to about amino acid residue 201, from about amino acid residue 1 to about amino acid residue 203, from about amino acid residue 1 to about amino acid residue 206; the I-BmoI nuclease domain and linker from about amino acid residue 1 to about amino acid residue 96, from about amino acid residue 1 to about amino acid residue 115, from about amino acid residue 1 to about amino acid residue 125, from about amino acid residue 1 to about amino acid residue 139, from about amino acid residue 1 to about amino acid residue 159, from about amino acid residue 1 to about amino acid residue 221, from about amino acid residue 1 to about amino acid residue 223, from about amino acid residue 1 to about amino acid residue 226; and the I-TulaI nuclease domain and linker from about amino acid residue 1 to about amino acid residue 114, and from about amino acid residue 1 to about amino acid residue 169.

As one of skill in the art will appreciate, the linking domain may be modified from a wild-type or native linking domain sequence. Suitable modifications include one or more amino acid substitutions, deletions or insertions, that do not impact on the function of the endonuclease, i.e. do not eliminate binding of the DNA-targeting domain to its substrate, nor eliminate nuclease activity. The native I-TevI linker has some DNA sequence preference. Accordingly, the present invention provides modified I-TevI linkers wherein the sequence of the native protein linker has been modified to change its DNA binding specificity, without affecting nuclease activity, to broaden or reduce targeting potential based on a specific target DNA sequence. Variant linking domains may comprise linking domain sequence to function effectively as a linking domain. Examples of at least about 30% sequence similarity with a native linking domain sequence, at least about 60-70%, and at least about 80%-90% or greater sequence similarity with a native linking domain to function as an effective linking domain. Suitable modifications include truncation of a native linking domain as set out above, and conservative amino acid substitutions as set out with respect to the nuclease domain.

The DNA-targeting domain may be any suitable domain that binds DNA in a site-specific manner. Examples of suitable DNA-targeting domains include, but are not limited to, the DNA binding domains of TAL-effector proteins, such as PthXol and AvrBs3 (from Xanthamonas campestris); zinc finger domains, e.g. ryA zinc finger binding domain and ryB zinc finger binding domain, and other distinct DNA-binding platforms, such as the binding domain in LADLIDADG homing endonucleases, e.g. I-OnuI, which have reprogrammable DNA-binding specificity similar to zinc fingers or TAL domains. A functionally equivalent variant binding domain based on a native binding domain, i.e. a binding domain which incorporates sequence modifications but which retains DNA binding activity, may also be utilized in the present chimeric endonuclease. Variant binding domains may comprise at least about 50% sequence similarity with a native binding domain sequence, at least about 60-70%, and at least about 80%-90% or greater sequence similarity with, a native binding domain to retain sufficient binding activity. Such a variant binding domain may include one or more of: an N- or C-terminal truncation, one or more amino acid substitutions, deletions or insertions, or modification of an amino acid, for example, modification of an amino acid sidechain entity. The DNA binding domain is typically bound at its N-terminal end to the linking domain or to the nuclease domain.

The targeting specificity of the present chimeric GIY-YIG endonuclease is a function of DNA-targeting domain and may be modified or enhanced by modifying the specificity of the DNA targeting domain as set out above. Additionally, for example, the specificity of the 3-zinc finger DNA-targeting domain of ryA or ryB may be enhanced by addition of zinc fingers to generate a 4-, 5-, or 6-zinc finger fusion protein.

In one embodiment, the DNA-targeting domain of a chimeric endonuclease is a TAL domain, or a modified TAL domain. Examples of suitable TAL domains are known in the art, for example US 2011/0301073 discloses Novel DNA-Binding Proteins and Uses Thereof and is specifically incorporated herein for its teaching of the structure of the DNA binding domain of TAL-effectors (i.e., TAL domain). A TAL domain is generally comprised of a plurality of repeat units that are typically 33 to 35 amino acid residue long segments and the repeats are typically 90-100% homologous to each other. Suitable repeats include, but are not limited to, those from Xanthomonas, for example, LTPEQVVAIASNIGGKQALETVQALLPVLCQAHG (SEQ ID NO:4), LTPDQVVAIASEGGGKQALETVQRLLPVLCQAHG (SEQ ID NO:5), and LTPEQVVAIASNIGGKQALETVQRLLPVLCQAHG (SEQ ID NO:6), those from Ralstonia solonacearum, for example, LTPQQWAIASNTGGKRALEAVCVQLPVLRAAPYR (SEQ ID NO:7), LSTEQWAIASNKGGKQALEAVKAHLLDLLGAPYV (SEQ ID NO:8) and LDTEQVVAIASHNGGKQALEAVKADLLDLRGAPYA (SEQ ID NO:9).

One suitable repeat sequence is L(T/P)(P/Q)(E/A/D/V)QVVAIASHDGGKQAL(E/A)T(V/M)QRLLPVLCQ(A/D)HG (SEQ ID NO: 10). The amino acid residues at positions 12 and 13 are referred to as a Repeat Variable Diresidue (RVD, residues HD in the sequence above) and determine the nucleic acid residue to which the repeat unit will bind. Thus, by selecting the sequence of RVDs and sequentially connecting repeat units comprising the RVDs, a TAL domain can be constructed that will bind to any desired sequence in the target DNA substrate, e.g. the binding site of the DNA targeting domain. For example, amino acid residues NI correspond to adenine, amino acid residues HD correspond to cytosine, amino acid residues NG correspond to thymine, amino acid residues NN correspond to guanine (and to a lesser degree adenine), amino acid residues HS correspond to A, C, T or G, amino acid residues N* (where * indicates a no amino acid residue) correspond to C or T, and amino acid residues HG correspond to T. Other RVDs are disclosed in US 2011/0301073 and are specifically incorporated herein by reference. Using the known DNA sequence of a gene, a chimeric endonuclease of the invention may be constructed specific to any gene locus. Examples of suitable gene loci include, but are not limited to, NTF3, VEGF, CCR5, IL2Rγ, BAX, BAK, FUT8, GR, DHFR, CXCR4, GS, Rosa26, AAVS 1 (PP1R1 2C), MHC genes, PITX3, ben-1, Pou5 F 1, (OCT4), C1, RPD1, and any other genes known to those skilled in the art.

A TAL domain may be constructed by fusing a plurality of repeat units. Any number of repeat units may fused to create a TAL domain, for example, from about 5 repeat units to about 30 repeat units, from about 5 repeat units to about 25 repeat units, from about 5 repeat units to about 20 repeat units, from: about 5 repeat units to about 15repeat units, or fern about 5 repeat units to about 10 repeat units, from about 7.5 repeat units to about 30 repeat units, from about 7.5 repeat units to about 25 repeat units, from about 7.5 repeat units to about 20 repeat units, from, about 7.5 repeat units to about 15 repeat units, or from about 7,5 repeat units to about 10 repeat units.

In some embodiments, a TAL domain of the invention, may comprise 5,6, 7, 8, 9, 10, 11, 12, 13, 14, 15, 16, 17, 18, 19, or 20 repeat units. In a given TAL domain, the repeat units typically share a high degree of homology. Thus, any two repeat units in a given TAL domain may be from about 75% to about 100%, from about 80% to about 100%, from about 85% to about 1.00%, from about 90% to about 100%, from about 91% to about 100%, from about 92% to about 100%, from about 93% to about 100%, from about 94% to about 100%, from about 95% to about 100%, from about 96% to about 100%, from about 97% to about 100%, from about 98% to about 100%, or from about 99% to about 100%, from about 75% to about 95%, from about 80% to about 95%, from about 91% to about 95%, from about 92% to about 95%, from about 93% to about 95%, from about 75% to about 90%, from about 80% to about 90%, from about 82% to about 90%, from about 84% to about 90%, from about 86% to about 90%, or from about 88% to about 90%, identical with each other.

TAL domains of the invention may also comprise one or more half repeats that are typically on either the N-terminal, the C-terminal, or on both the N- and C-terminals of the TAL domain. In other embodiments, at least one repeat unit is modified at some or all of the amino acids at positions 4, 11, 12, 13 or 32 within the repeat unit. In some embodiments, at least one repeat unit is modified at 1 or more of the amino acids at positions 2, 3, 4, 11, 12, 13, 21, 23, 24, 25, 26, 27, 28, 30, 31, 32, 33, 34, or 35 within one repeat unit.

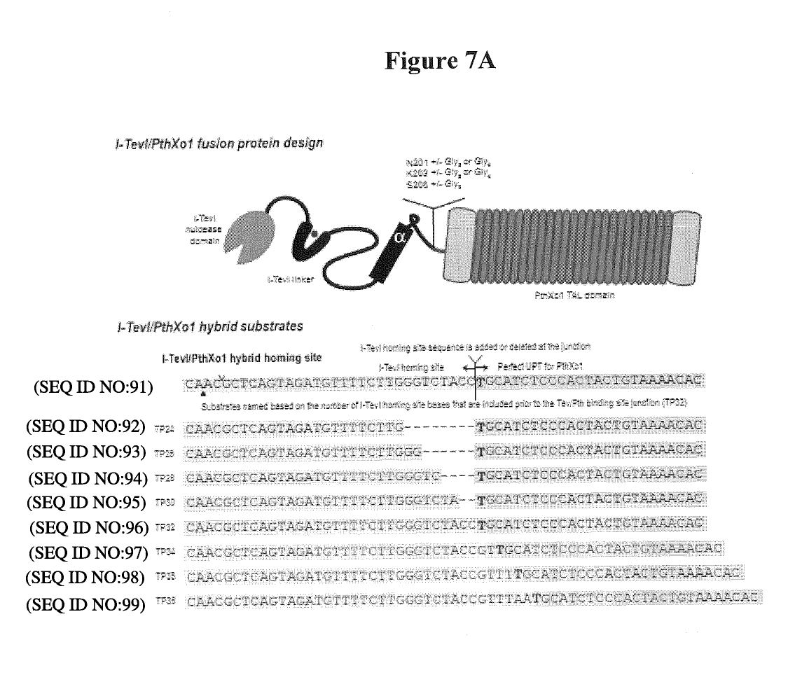

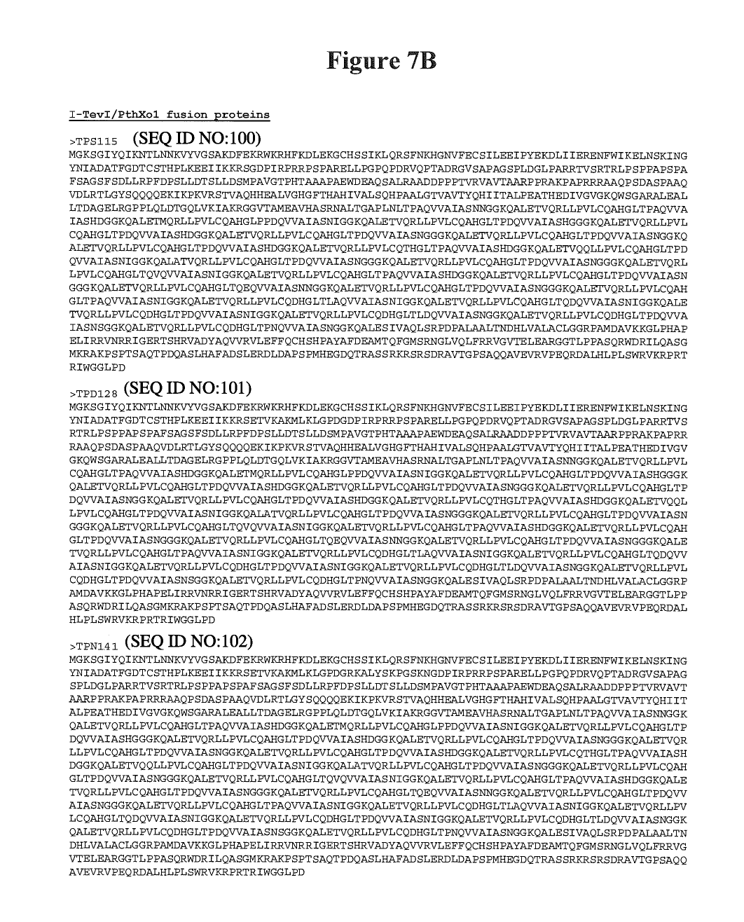

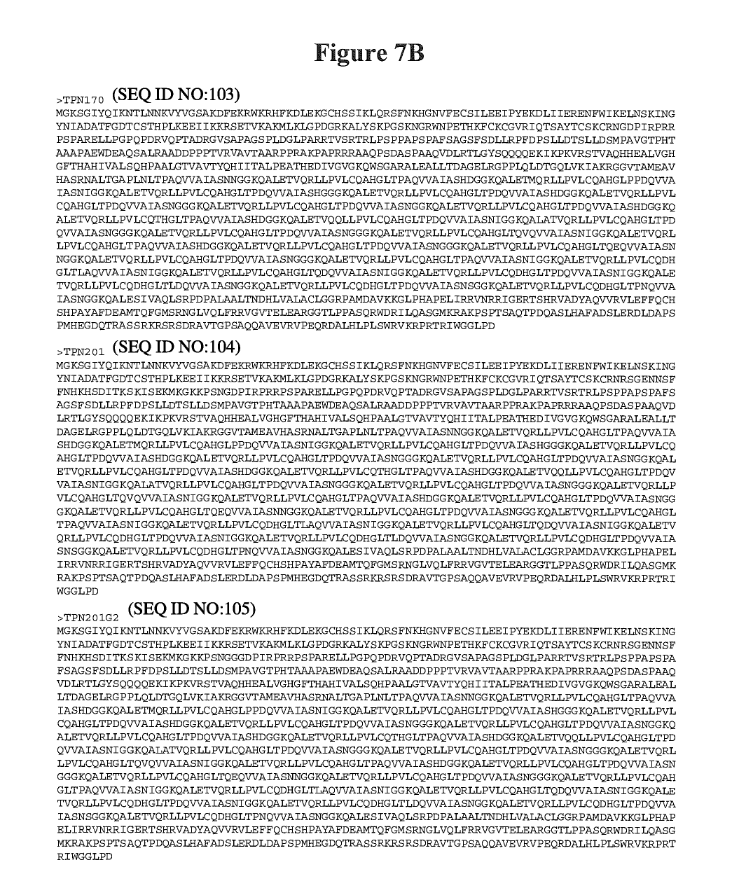

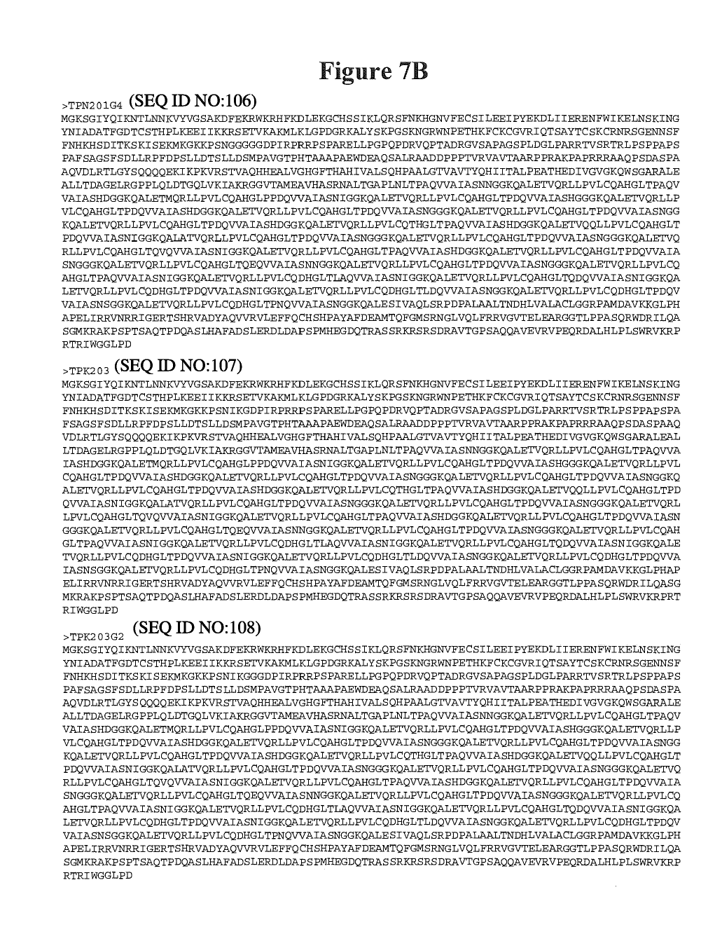

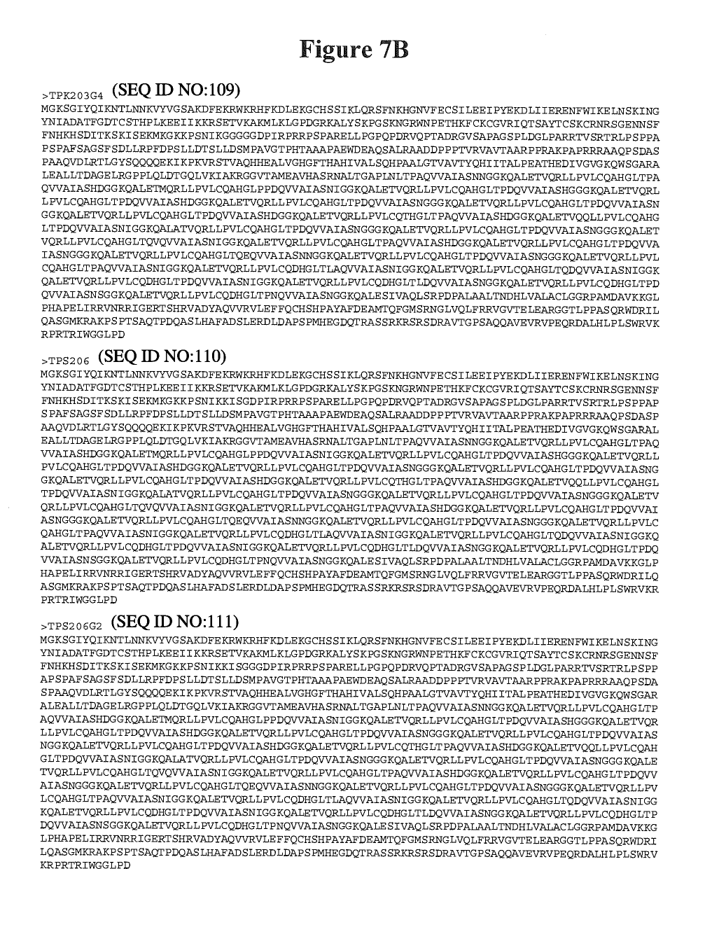

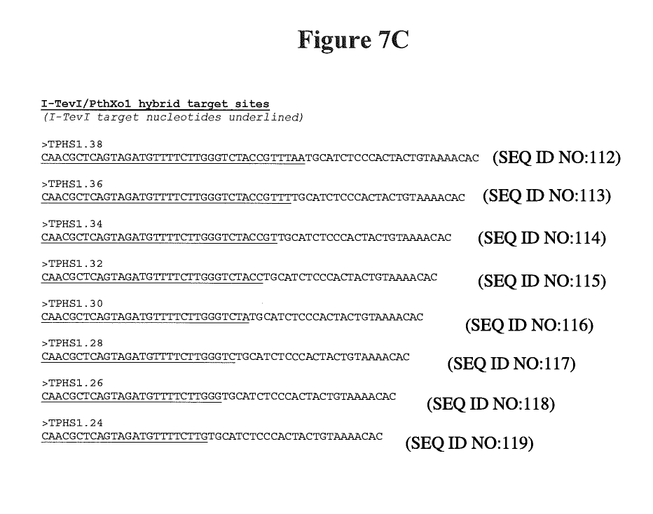

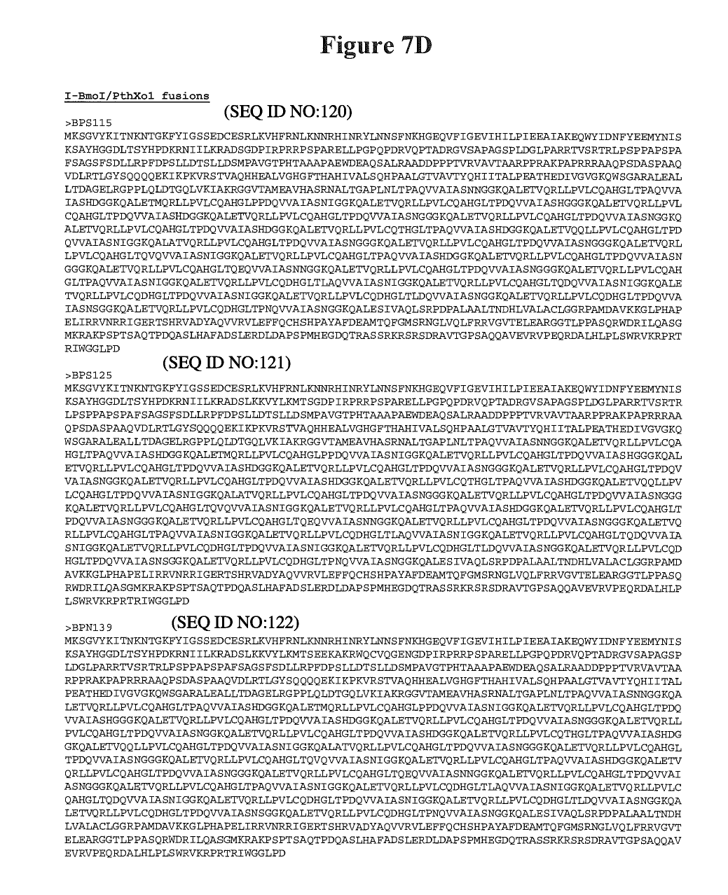

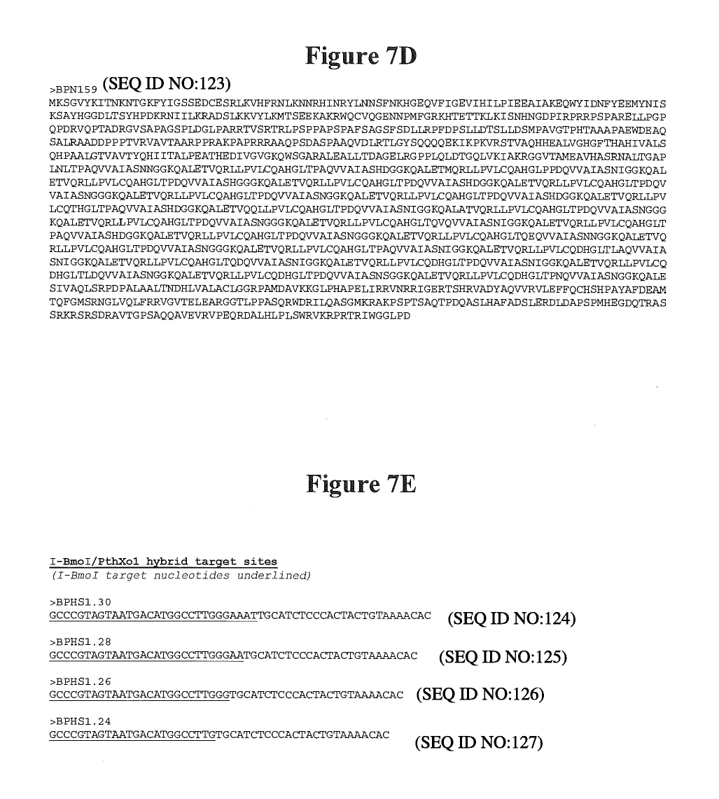

In addition to the repeat ends described above, a TAL domain of the invention may also comprise flanking sequences at the N- and/or C-terminal of the TAL domain. The flanking sequences may be of any length that does not interfere with the DNA-binding of the TAL domain. Flanking sequences may be from about 1 amino acid residue to about 300 amino acid residues, from about 1 amino acid residue to about 250 amino acid residues, from about 1 amino acid residue to about 200 ammo acid residues, from about 1 amino acid residue to about 150 amino acid residues, from about 1 amino acid residue to about 125 amino acid residues, from about 1 amino acid residue to about 100 ammo acid residues, from, about 1 amino acid residue to about 75 amino acid residues, from about 1 amino acid residue to about 50 amino acid residues, from about 1 amino acid residue to about 40 amino acid residues, from about 1 amino acid residue to about 30 amino acid residues, from about 1 amino acid residue to about 20 amino acid residues, or from about 1 amino acid residue to about 10 amino acid residues. The flanking sequences may be of any amino acid sequence. In some embodiments, the flanking sequences may be derived from the naturally occurring sequence of a TAL-effector protein, which may be the same or different TAL-effector protein from which the repeat units are derived. Thus, the present invention encompasses TAL domains comprising repeat units having an amino acid sequence found in a first TAL-effector protein and one or more flanking sequences found in a second TAL-effector protein. One suitable source for flanking sequences is amino acid residues 130 to 416 of SEQ ID NO:101 which is the N-terminal flanking region of PthXol (FIG. 7A). In some embodiments, a flanking sequence may comprise all or a part of amino acid residues 130 to 416 of SEQ ID NO: 101. For example, a flanking sequence may comprise from about amino acid residue 150 to about amino acid residue 416, from about amino acid residue 175 to about amino acid residue 416, from about amino acid residue 200 to about amino acid residue 416, from about amino acid residue 225 to about amino acid residue 416, from about amino acid residue 250 to about amino acid residue 416, from about amino acid residue 275 to about amino acid residue 416, from about amino acid residue 300 to about amino acid residue 416, from about amino acid residue 325 to about amino acid residue 416, from about amino acid residue 350 to about amino acid residue 416, from about amino acid residue 375 to about amino acid residue 416, or from about amino acid residue 400 to about amino acid residue 416. In some embodiments, a flanking sequence may have sequence identity with one or more of the flanking sequence above. For example, a flanking sequence may comprise a sequence that is from about 80% to about 100% identical to the sequence of from about amino acid residue 350 to about amino acid residue 416, from about 85% to about 100% identical, from about 90% to about 100% identical, from about 95% to about 100% identical, from about 80% to about 95% identical, from about 80% to about 90% identical, or from about 80% identical to about 85% identical. A flanking sequence may comprise a sequence that is from about 80% to about 100% identical to the sequence of from about amino acid residue 300 to about amino acid residue 416, from about 85% to about 100% identical, from about 90% to about 100% identical, from about 95% to about 100% identical, from about 80% to about 95% identical, from about 80% to about 90% identical, or from about 10% identical to about 85% identical. A flanking sequence may comprise a sequence that is from about 80% to about 100% identical to the sequence of from about amino acid residue 250 to about amino acid residue 416, from about 85% to about 100% identical from about 90% to about 100% identical, from about 95% to about 100% identical, from about 80% to about 95% identical, from about 80% to about 90% identical, or from about 80% identical to about 85% identical.

Suitable modified TAL domains may include one or more amino acid deletions, insertions or substitutions which do not eliminate the DNA binding activity thereof, for example, modifications at one or more amino acid residues other than amino acid residues at position 12 and 13, such as those indicated with multiple amino acid residues in parenthesis in the above sequence. Other proteins having TAL domains can be used to identify suitable repeats that can be used to construct a DNA-targeting domain. Examples include, but are not limited to, Avrb6 from Xanthomonas citri subsp. Malvacearum GenBank accession number AAB00675.1, PthN from Xanthomonas campestris GenBank accession number AAB69865.1, PthA from Xanthomonas citri GenBank accession number AAC43587.1, avirulence protein from Xanthomonas oryzae pv. Oryzae GenBank accession number AAB98343.1, AvrXa7 from Xanthomonas oryzae pv. Oryzae GenBank accession number AAG02079.2, AvrXa3 from Xanthomonas oryzae pv. Oryzae GenBank accession number AAN01357.1, AvrXa5 from Xanthomonas oryzae pv. Oryzae GenBank accession number AAQ79773.2, PthXo3 from Xanthomonas oryzae pv. Oryzae GenBank accession number AAS46027.1, and PthXo4 from Xanthomonas oryzae pv. Oryzae GenBank accession number AAS58127.2. The sequence of each of these proteins is specifically incorporated herein by reference.

Chimeric endonucleases of the invention comprising a TAL domain may be constructed using techniques well known in the art. One suitable protocol is found is Sanjana Nature Protocols 7:171-192 (2012) which is specifically incorporated herein by reference. To prepare a TAL domain, nucleic acid encoding each desired repeat unit may be amplified with ligation adapters that uniquely specify the position of the repeat unit in the TAL domain to create a library that can be reused. Appropriate amplification products may be ligated together into hexamers and then amplified by PCR. The hexamers may be assembled into a suitably prepared plasmid background, for example, using a Golden Gate digestion-ligation. The plasmid backbone may contain a negative selection gene, for example, ccdB, which selects against empty plasmid. The plasmid may be constructed to contain coding sequence for one or more flanking sequences such that insertion of the coding sequence for the TAL domain will be in frame with the flanking sequences resulting in TAL domain comprising flanking sequences. The TAL domain coding sequences, optionally with flanking sequences, can then be combined with the nuclease coding sequences and any other desired coding sequences, for example, nuclear localization sequences (NLS), using standard techniques. Suitable nuclear localization sequences are known in the art. Examples include, but are not limited to, the nucleoplasmin NLS KRX₁₀KKKL (SEQ ID NO:11) (Moore J D, J Cell Biol. 1999 Jan. 25; 144,213-24), the SV40 LargeT antigen NLS PKKKRKV (SEQ ID NO:12) (Kalderon D., Cell., 1984,39,499-509), the BRCA1 NLS PKKNRLRRP (SEQ ID NO:13) (Chen C F, J.Biol.Chem. 1996,271,32863-32868) and the c-myb NLS PLLKKIKQ (SEQ ID NO:14) (Dang and Lee, J Biol Chem, 1989,264,18019).

Chimeric endonucleases of the invention may optionally comprise one or more functional domains. Suitable functional domains include, but are not limited to, transcription factor domains (activators, repressors, co-activators, co-repressors), additional nuclease domains, silencer domains, oncogene domains (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases), DNA targeting enzymes such as transposons, integrases, recombinases and resolvases and their associated factors and modifiers, nuclear hormone receptors, and ligand binding domains.

Examples of chimeric endonucleases include, but are not limited to, TevI nuclease linked to PthXol TAL DNA targeting domain, I-TevI nuclease linked to ryA or ryB zinc finger DNA targeting domain, I-TevI nuclease linked to OnuI DNA targeting domain, I-BmoI nuclease linked to PthXol TAL DNA targeting domain, I-BmoI nuclease linked to ryA or ryB zinc finger DNA targeting domain, I-Tula1 linked to ryA or ryB zinc finger DNA targeting domain, Tula linked to a PthXol TAL DNA-targeting domain, and Tula linked to the I-OnuI targeting domain. Nucleases may be linked via a linking domain as described above, either the linking domain native to the nuclease or derived from the linking domain native to the nuclease, or a linking domain of a different nuclease or derived from a different nuclease, or a linking domain comprising a random sequence.

The present chimeric peptides may be made using well-established peptide synthetic techniques, for example, FMOC and t-BOC methodologies. In addition, polynucleotides disclosed herein, for example, DNA substrates and DNA encoding the present chimeric eudonucleases may also be made based on the known sequence information using well-established techniques. Peptides and oligonucleotides are also commercially available.

Recombinant technology may also be used to prepare the chimeric endonuclease. In this regard, a DNA construct comprising DNA encoding the selected nuclease, linking domain (if present), DNA-targeting domain, and any functional domains if present may be inserted into a suitable expression vector which is subsequently introduced into an appropriate host cell (such as bacterial, yeast, algal, fungal, insect, plant and mammalian) for expression. Such transformed host cells are herein characterized as having the chimeric endonuclease DNA incorporated “expressibly” therein. Suitable expression vectors are those vectors which will drive expression of the inserted DNA in the selected host. Typically, expression vectors are prepared by site-directed insertion of a DNA construct therein. The DNA construct is prepared by replacing a coding region, or a portion thereof, within a gene native to the selected host, or in a gene originating from a virus infectious to the host, with the endonuclease construct. In this way, regions required to control expression of the endonuclease DNA, which are recognized by the host including a promoter and a 3′ region to terminate expression, are inherent in the DNA construct. To allow selection of host cells stably transformed with the expression vector, a selection marker is generally included in the vector which, takes the form of a gene conferring some survival advantage on the transformants such as antibiotic resistance. Cells stably transformed with endonuclease DNA-containing vector are grown in culture media and under growth conditions that facilitate the growth of the particular host cell used. One of skill in the art would be familiar with the media and other growth conditions

The utility of a chimeric endonuclease in accordance with the invention may be confirmed using a DNA subsume designed for the endonuclease. The DNA substrate will include suitable counterpart regions to the nuclease, linking and DNA-targeting domains of the endonuclease. Thus, the substrate will include a cleavage motif of the nuclease domain, a DNA spacer that correlates with the linking domain and a binding site for the DNA-targeting domain. For example, for a chimeric endonuclease including, the I-TevI nuclease domain, at least a portion of the I-TevI linker as the linking domain and the DNA-targeting domain of a zinc finger (e.g. of ryA or ryB), a suitable substrate will include a cleavage motif of I-TevI (5′-CNNNG-3), a binding site for the selected zinc finger and a DNA spacer that connects the two and which is compatible with the I-TevI linker to permit interaction between the nuclease and the substrate. It will be appreciated that the substrate may incorporate a native cleavage motif or may incorporate a cleavage motif derived from the native cleavage motif, i.e. somewhat modified from the native cleavage motif while still recognized and cleaved by the nuclease. The binding site for the DNA-targeting domain may similarly be a native sequence, or may be modified without loss of function. Between the cleavage motif and the binding site for the DNA-targeting domain there may be a DNA spacer. The DNA spacer will be of a size that permits binding of the endonuclease DNA-targeting domain to the substrate binding site, and nuclease access to the cleavage motif. Generally the DNA spacer that links the cleavage motif to the binding site may comprise about 10 to about 30 base pairs, and typically comprises about 15-25 base pairs. The length of the DNA spacer may be adjusted depending on the length of the linker domain and any flanking sequences present in the chimeric endonuclease of the invention. For applications where a chimeric endonuclease of the invention is to target a DNA in a cell, it is not possible to adjust the DNA spacer length. Instead, the length of the linker may be adjusted such that, upon binding of the DNA-targeting domain to the DNA, the nuclease domain is brought into proximity with the cleavage site.

A given DNA substrate is useful in a method of determining the activity of its corresponding chimeric endonuclease. In this regard, the DNA substrate may be utilitized as pair of complementary oligonucleotides annealed together, which may be detectably labeled, e.g. radioactively labeled. To assay for the activity of a selected chimeric endonuclease, the endonuclease is incubated with its substrate under conditions suitable to permit binding of the endonuclease DNA targeting domain to the binding site on the substrate, and subsequent nuclease cleavage at the cleavage site. Cleavage of the substrate can then be determined using well-established techniques, for example, polyacrylamide gel electrophoresis.

Alternatively, the DNA substrate may be incorporated within a vector for use in an assay to determine endonuclease activity. In one embodiment, a cell-based bacterial Escherichia coli two-plasmid genetic selection system may be utilised to determine whether or not the chimeric endonuclease can cleave the target cleavage site. The DNA encoding the chimeric endonuclease is incorporated and expressed from one plasmid of the system, and the target DNA substrate is incorporated and expressed from the second plasmid. The target substrate plasmid also encodes a toxin, such as a DNA gyrase toxin. If the expressed endonuclease cleaves the target site, the toxin will not be expressed and the cells, e.g. bacterial ceils such as E. coli cells, will survive when plated on selective solid, media, plates. If the endonuclease cannot cleave the target site, the toxin will be expressed and the cells will not survive on selective media plates. The percentage survival for each combination of fusion, and target, site is simply the ratio of survival on selective to non-selective plates.

In another embodiment, a yeast-based assay is provided which utilizes detectable enzyme activity, e.g. beta-galactosidase activity as a readout of endonuclease activity. The lacZ gene is disrupted and partially duplicated in a first plasmid. The DNA substrate is cloned in between the lacZ gene fragments. Cleavage of the substrate by the endonuclease (expressed from a second plasmid) initiates DNA repair and generation of a functional LacZ protein (and beta-galactosidase activity).

In another embodiment, a mammalian cell-based assay is provided which utilizes detectable activity, e.g. the fluorescence of green fluorescent protein (GFP), as a readout of endonuclease activity. The GFP gene is disrupted and partially duplicated in a first plasmid. The DNA substrate is cloned in between the GFP gene fragments. Cleavage of the substrate by the endonuclease (expressed from a second plasmid) initiates DNA repair and generation of a functional GFP and fluorescence can be detected.

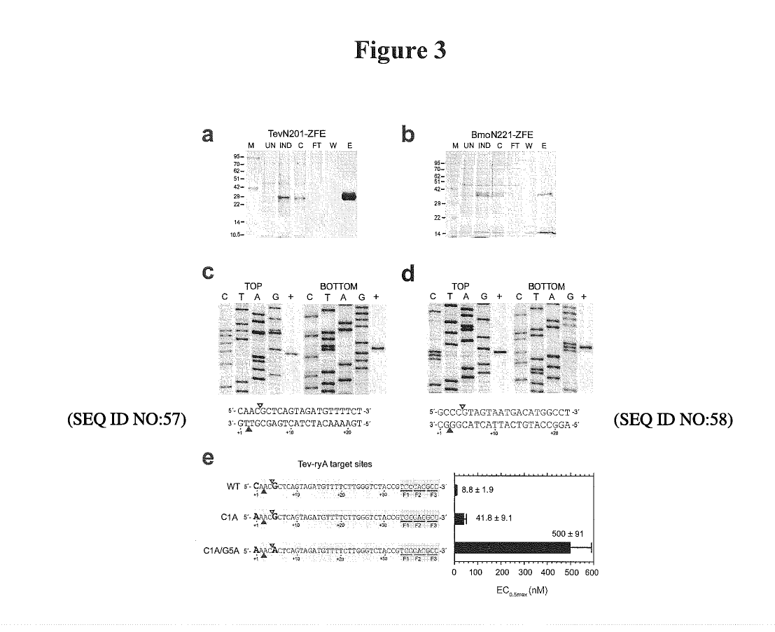

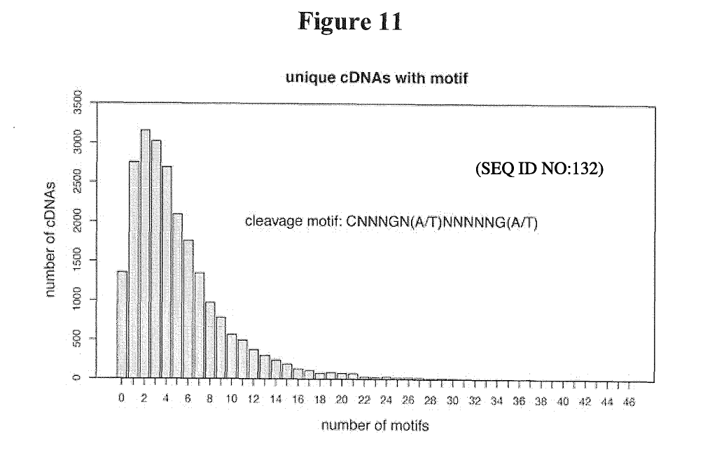

The present invention also provides methods for detection of the presence or absence of single nucleotide polymorphisms in a target DNA. In some embodiments, chimeric endonucleases of the invention comprise a nuclease domain that recognises a 5′CNNNG3′ cleavage motif and do not cleave, or cleave at a much reduced level, DNA sequences in which this motif has been altered. See FIG. 3e. As shown in FIG. 11, the motif is prevalent in human cDNA sequences. Where one allele of a SNP comprises a functional motif and other alleles have a non-functional motif, this difference in reactivity can be used to identify which allele is present in a given sample. This could be useful for high throughput SNP screening for specific disease causing alleles.

Thus, in a further embodiment of the invention, a kit comprising a chimeric endonuclease and a DNA substrate therefor is provided. Alternatively, a kit including a chimeric endonuclease-encoding plasmid and a substrate-encoding plasmid that expresses a cleavage-dependent marker, or that results in cleavage-dependent cell survival. In some embodiments, kits of the invention may comprise a second plasmid with reporter gene and the DNA binding motif—optimized DNA spacer—and cleavage site. In combination with a chimeric endonuclease of the invention such a plasmid may be used to identify optimized endonuclease—linker—DNA binding domain constructs. In some embodiments, plasmids in kits of the invention may comprise one or more multicloning sites (MCS) that may be disposed in such a fashion as to permit rapid exchange of nuclease and/or DNA targeting domains. For example, a plasmid may contain MCS-universal linker-MCS. In some embodiments, kit of the invention may comprise a plasmid encoding an I-TevI-Tal domain chimeric endonuclease. A chimeric endonuclease thus encoded may comprise a linker domain disposed between the nuclease and DNA-targeting domain as well as one or more other functional domains, for example, nuclear localisation signals, disposed at either the N- or C-terminal or both.

The present chimeric GIY-YIG endonucleases are active in vivo and in vitro, function as monomers, and retain the cleavage specificity associated with the parental GIY-YIG nuclease domain. The GIY-YIG nuclease domain is shown to be a viable alternative m the FokI nuclease domain for genome editing applications.

The present invention provides materials and methods for manipulating the genome of a target organism, for example, by disabling one or more genes and/or by changing the nucleic acid sequence of the gene. As used herein, a gene includes a DNA region, encoding a gene product (which may be a protein or an RNA), as well as all DNA regions which regulate the production of the gene product which may include, but are not limited to, one or more of promoter sequences, terminators, translational regulatory sequences such as ribosome binding sites and internal ribosome entry sites, enhancers, silencers, insulators, boundary elements, replication origins, matrix attachment sites and locus control regions.

Methods of the invention typically include introducing one or more chimeric endonucleases and/or nucleic acid molecules encoding such chimeric endonucleases, into one or more cells, which may be isolated or may be part of an organism. Any method of introducing known to those skilled in the art may be used. Examples include direct injection of DNA and/or RNA encoding chimeric endonucleases of the invention, transfection, electroporation, transduction, lipofection and the like. Suitable cells include, but are not limited to, eukaryotic and prokaryotic cells. Cells may be cultured cell lines or primary cells. Primary cells will typically be used when it is desired to modify the cell and reintroduce it into the organism from which it was derived. Cells may be from any type of organism, for example, may be mammalian cells, plant cells, insect cells, or fungal cells. Suitable types of cell include, but are not limited to, stem cells (e.g., embryonic stem cells, induced pluripotent stem cells, hematopoietic stem cells, neuronal stem cells, mesenchymal stem cells, muscle stem cells and skin stem cells). In some embodiments, the cells used in the methods of the invention may be plant cells. In addition to the methods of introducing nucleic acids into cells described above, DNA constructs encoding chimeric endonucleases of the invention may be introduced into plant cells using Agrobacterium tumefaciens-mediated transformation. Suitable plant cells include, but are not limited to, cells of monocotyledonous (monocots) or dicotyledonous (dicots) plants, plant organs, plant tissues, and seeds. Examples of plant species of interest include, but are not limited to, corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Penniserum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. Turgidum ssp. durum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidnum guajava), mango (Mangifera indica), olive (Olea europaea), papaya (Carica papaya), cashew (Anacardium occidentale), macadamia (Macadamia integrifolia), almond (Prunnus amygdalus), sugar beets (Beta vulgaris), sugarcane (Saccharum spp.), coats, barley, vegetables, ornamentals, and conifers. In some embodiments, plants for use in methods of the present invention are crop plants (for example, sunflower, Brassica sp., cotton, sugar beet, soybean, peanut, alfalfa, safflower, tobacco, corn, rice, wheat, rye, barley triticale, sorghum, millet, etc.). Plant cells may be from any part of the plant and/or from any stage of plant development. In some embodiments, suitable plant cells are those that may be regenerated into plants after being modified using the methods of the invention, for example, cells of a callus. Methods of the invention may also include introducing one or more chimeric endonucleases and/or nucleic acid molecules encoding such chimeric endonucleases, into one or more algal cells. Any species of algae may be used in the methods of the invention. Suitable examples include, but are not limited to, algae of the genus Skeletonema, Thalassiosira, Phaeodactylum, Chaetoceros, Cylindrotheca, Bellerochea, Actinocyclus, Nitzchia, Cyclotella, Isochrysis, Pseudoisochrysis, Dicrateria, Monochrysis, (Pavlova), Tetraselmis (Platymonas), Pyramimonas, Micromonas, Chroomonas, Cryptomonas, Rhodomonas, Chlamydomonas Chlorococcum, Olisthodiscus, Carteria, Dunaliella, or Spirulina. Other examples include Haematococcus pluvialis, Chlorella vulgaris, and the halophilic algae Dunaliella sp.

The present invention provides methods of inactivating a gene. Such methods typically comprise introducing a nucleic acid molecule encoding a chimeric endonuclease of the invention into a cell under conditions causing the expression of the chimeric endonuclease. The chimeric endonuclease of the invention can comprise a DNA-targeting domain selected to bind to a gene of interest. The chimeric endonuclease of the invention can cleave the gene of interest leaving a double-stranded break. The normal repair functions in the cell will result in the production of some inserted or deleted bases, which may result in a frame shift thereby inactivating the gene. In some embodiments, the chimeric endonuclease may be transiently introduced into the cell. This may be accomplished by transfecting a plasmid with a promoter controlling the expression of the chimeric endonuclease that does not drive expression unless induced, for example, the Tet-On promoter. Alternatively, transient expression may be accomplished by introducing mRNA encoding the chimeric endonuclease of the invention into the cell. Normal housekeeping functions of the cell will degrade the mRNA over time thereby stopping expression of the chimeric endonuclease.

Methods of the invention also include methods of changing the nucleic acid sequence of a gene. Typically a nucleic acid molecule encoding a chimeric endonuclease of the invention is introduced into a target cell under conditions causing the expression of the chimeric endonuclease. The chimeric endonuclease of the invention is constructed so as to bind to and cleave a gene of interest. In addition, a second nucleic acid molecule comprising a region having a nucleotide sequence that has a high degree of sequence identity to the gene in the region of the cleavage site is introduced into the cell. The region of high sequence identity may have a length of from about 10 basepairs to about 1000 basepairs, from about 25 basepairs to about 1000 basepairs, from about 50 basepairs to about 1000 basepairs, from about 75 basepairs to about 1000 basepairs from about 100 basepairs to about 1000 basepairs, from about 200 basepairs to about 1000 basepairs, from, about 300 basepairs to about 1000 basepairs, from about. 400 basepairs to about 1000 basepairs, from about 500 basepairs to about 1000 basepairs, from about 250 basepairs to about 1000 basepairs, from about 10 basepairs to about 500 basepairs, from about 25 basepairs to about 500 basepairs, from about 50 basepairs to about 500 basepairs, from about 75 basepairs to about 500 basepairs from about 100 basepairs to about 500 basepairs, from about 200 basepairs to about 500 basepairs, from about 300 basepairs to about 500 basepairs, from about 400 basepairs to about 500 basepairs, from about 10 basepairs to about 250 basepairs, from about 25 basepairs to about 250 basepairs, from about 50 basepairs to about 250 basepairs, from about 75 basepairs to about 250 basepairs from about 100 basepairs to about 250 basepairs, from about 150 basepairs to about 250 basepairs, or from about 200 basepairs to about 250 basepairs, corresponding to regions in the gene located both 5′ and 3′ to the anticipated cleavage site. High sequence identity means the region and the corresponding region in the gene nave a sequence identity of from about 80% to about 100%, from about 82% to about 100%, from about 86% to about 100%, from about 88% to about 100%, from about 90% to about 100%, from about 92% to about 100%, from about 94% to about 100%, from about 90% to about 100%, from about 98% to about 100%, or from about 80% to about 95%, from about 82% to about 95%, from about 80% to about 95%, from about 88% to about 95%, from about 90% to about 95%, from about 92% to about 95%, or from about 80% to about 90%, from about 82% to about 90%, from about 86% to about 90%, from about 88% to about 90%. The region may comprise an altered sequence when compared to the gene of interest, for example, may have one or more mutations that will result in changes to one or mom amino acids in a protein encoded by the gene. The double-stranded break introduced by the chimeric endonuclease of the Invention may he repaired by homologous recombination with the region of high sequence identity of the second nucleic acid, effectively substituting all or a portion of the sequence of the homologous region in the second nucleic acid molecule for the original sequence of the gene. This results in a gene with modified nucleic acid sequence. In some embodiments, the chimeric endonuclease of the invention is transiently expressed in the cell. This may be accomplish by transfecting a plasmid with a promoter controlling the expression of the chimeric endonuclease that docs not drive expression unless induced, for example, the Tet-On promoter. Alternatively, transient expression may be accomplished by introducing mRNA encoding the chimeric endonuclease of the invention into the cell. Normal housekeeping functions of the cell will degrade the mRNA over time thereby stopping expression of the chimeric endonuclease. In some embodiments, the second nucleic acid molecule may be a linear DNA molecule.

Methods of the invention also include methods of deleting all or a portion of the nucleic acid sequence of a gene. Typically a nucleic acid molecule encoding a chimeric endonuclease of the invention is introduced into a target cell under conditions causing the expression of the chimeric endonuclease. The chimeric endonuclease of the invention is constructed so as to bind to and cleave a gene of interest. In addition, a second nucleic acid molecule comprising a region, having a nucleotide sequence that has a high degree of sequence Identity to the gene in the region of the cleavage site is introduced into the cell. The region of high sequence identity is as described above except that the region will lack sequence corresponding to the portions of the gene adjacent to the anticipated cleavage site. After homologous recombination between the gene and the second nucleic acid molecule, the lacking sequence will appear as a deletion of the sequence of the gene. Any number of basepairs may be lacking, from 1 to the entire sequence of the gene. The double strand break introduced by the chimeric endonuclease of the invention may be repaired by homologous recombination with the region of high sequence identity of the second nucleic acid, effectively substituting all or a portion of the sequence of the region of high sequence identity for the original sequence of the gene. Since this region contains a deletion at the cleavage site of the chimeric endonuclease of the invention, this results in a gene with a deletion in its nucleic acid sequence. In some embodiments, the chimeric endonuclease of the invention is transiently expressed in the cell. This may be accomplished by transfecting a plasmid with a promoter controlling the expression of the chimeric endonuclease that does not drive expression unless induced, for example, the Tet-On promoter. Alternatively, transient expression may be accomplished by introducing mRNA encoding the chimeric endonuclease of the invention into the cell. Normal housekeeping functions of the cell will degrade the mRNA over time thereby stopping expression of the chimeric endonuclease. In some embodiments, the second nucleic acid molecule may be a linear DNA molecule.

Methods of the invention also include methods of making a cell having an altered genome. In some embodiments, the altered genome may comprise an inactivated gene. In some embodiments, the altered genome may comprise a gene having one or more mutations. In some embodiments the altered genome may lack all or a portion of a gene. Typically a nucleic acid molecule encoding a chimeric endonuclease of the invention is introduced into a target cell under conditions causing the expression of the chimeric endonuclease. The chimeric endonuclease of the invention is constructed so as to bind to and cleave a gene of interest. Cleavage of the target and DNA repair will result in an inactivated gene. In embodiments where the altered genome comprises a mutated gene, a nucleic acid molecule encoding a chimeric endonuclease of the invention is introduced into a target cell under conditions causing the expression of the chimeric endonuclease. In addition, a second nucleic acid molecule comprising a region having a nucleotide sequence that has a high degree of sequence identity to the gene in the region of the cleavage site is introduced into the cell. The region is as described above. The region may comprise an altered sequence when compared to the gene of interest, for example, may have one or more mutations that will result in changes to one or more amino acids in a protein encoded by the gene. The double-stranded break introduced by the chimeric endonuclease of the invention may be repaired by homologous recombination with the region of high sequence identity of the second nucleic acid, effectively substituting all or a portion of the sequence of the region of high sequence homology in the second nucleic acid molecule for the original sequence of the gene. This results in a cell with an altered genome. In embodiments wherein the altered genome lacks all or a portion of a gene, a nucleic acid molecule encoding a chimeric endonuclease of the invention is introduced into a target cell under conditions causing the expression of the chimeric endonuclease. The chimeric endonuclease of the invention is constructed so as to bind to and cleave a gene of interest. In addition, a second nucleic acid molecule comprising a region having a nucleotide sequence that has a high degree of sequence identity to the gene in the region of the cleavage site is introduced into the cell. The region typically lacks the sequence of the gene adjacent to the cleavage site, i.e. has a deletion that encompasses the anticipated cleavage site. The double-stranded break introduced by the chimeric endonuclease of the invention may be repaired by homologous recombination with the region of high sequence identity of the second nucleic acid, effectively substituting all or a portion of the sequence of the region for the original sequence of the gene. Since this region contains a deletion at the cleavage site of the chimeric endonuclease of the invention, this results in a gene with a deletion in its nucleic-acid sequence. In some embodiments, the chimeric endonuclease of the invention is transiently expressed in the cell. This may be accomplished by transfecting a plasmid with a promoter controlling the expression of the chimeric endonuclease that does not drive expression unless induced, for example, the Tet-On promoter. Alternatively, transient expression may be accomplished by introducing mRNA encoding the chimeric endonuclease of the invention into the cell. Normal housekeeping functions of the cell will degrade the mRNA over time thereby stopping expression of the chimeric endonuclease. In some embodiments, the second nucleic acid molecule may be a linear DNA molecule.

Chimeric endonucleases of the invention may be used for in biological research by providing a mechanism to manipulate the genome of a cell or organism. Such genome editing allows the elucidation of the role of individual genes and portions of genes by allowing the controlled introduction of changes into the genome. This will allow the production of customised cells that are suitable for use in screening. The present invention also permits gene therapy, for example, by correcting a genetic defect using the materials and methods described herein. The present methods are particularly well suited for ex vivo methods of gene therapy where cells are removed from a patient, manipulated to achieve a desired outcome, and reintroduced in the patient. Materials and methods of the invention will find use in agricultural for creation of plants having improved growth rate, tolerance to stresses such as drought and pests, and taste. Materials and methods of the invention will find application in molecular biology and diagnostics by allowing the direct manipulation of any desired target DNA.

Embodiments of the invention are described by reference to the following specific examples.

### EXAMPLE 1

### Materials and Methods

### Bacterial Strains and Plasmid Construction

Escherichia coli strains DH5α and ER2566 (New England Biolabs) were used for plasmid manipulations and protein expression, respectively. E. coli strain BW25141(λDE3) was used for genetic selection assays. A complete description of all plasmids used in this study are listed in Table 1, and oligonucleotides are listed in Table 2.

TABLE 1
Strains and plasmids used in this study.

| Strains | Description | Source |
|---|---|---|
| DH5α | F⁻, φ80dlacZΔM15, Δ(lacZYA-argF)U169, deoR, recA1, endA1, hsdR17(rk⁻, | Invitrogen |
| | mk⁺), phoA, supE44, λ⁻, thi-1, gyrA96, relA1 | |
| ER2566 | F− λ− fhuA2 [lon] ompT lacZ::T7 gene 1 gal sulA11 Δ(mcrC-mrr)114::IS10 | N.E.B. |
| | R(mcr-73::miniTn10-TetS)2 R(zgb-210::Tn10)(TetS) endA1 [dcm] | |
| BW25141(λDE3) | F⁻ lacI^{q} rrnB_{T14} DlacZ_{WJ16} DphoBR580 hsdR514 DaraBAD_{AH33} | Ref 1, 2 |
| | DrhaBAD_{LD78} galU95 endA_{BT333} uidA(DMluI)::pir+ recA1, λDE3 lysogen | |
| pACYCDuet-1 | ori_{p15A}, cm | Novagen |
| p11-lacY-wtx1 | ori_{pBR322}, amp | Ref 1 |
| pSP72 | ori_{pBR322}, amp | Promega |
| LITMUS28i | ori_{pMB1}, amp | N.E.B. |
| pACYCIBmoI | pACYCDuet-1, containing the 798bp codon optimized I-BmoI gene in the | Ref 2 |
| | NdeI and XhoI sites | |
| pryAzf | ori_{pUC}, kan | I.D.T. |
| pACYCryAZf + H | pACYCDuet-1, containing the ryA zinc-finger gene with a c-terminal 6- | This study |
| | histidine tag cloned into the BamHI and XhoI sites | |
| pACYCryAZf | pACYCDuet-1, containing the ryA zinc-finger gene cloned into the BamHI | This study |
| | and XhoI sites | |
| pTevN201-ZFE (or +H) | pACYCryAZf (or +H), with residues 1-N201 of I-TevI (DE832/840) cloned | This study |
| | into the NcoI and BamHI sites (+/−6xHis) | |
| pTevN201G₂-ZFE (or +H) | pACYCryAZf (or +H), with residues 1-N201 of I-TevI (DE833/840) + 2 | This study |
| | glycine residues cloned into the NcoI and BamHI sites (+/−6xHis) | |
| pTevN201G₄-ZFE (or +H) | pACYCryAZf (or +H), with residues 1-N201 of I-TevI (DE834/840) + 4 | This study |
| | glycine residues cloned into the NcoI and BamHI sites (+/−6xHis) | |
| pTevK203-ZFE (or +H) | pACYCryAZf (or +H), with residues 1-K203 of I-TevI (DE835/840) cloned | This study |
| | into the NcoI and BamHI sites (+/−6xHis) | |
| pTevK203G₂-ZFE (or +H) | pACYCryAZf (or +H), with residues 1-K203 of I-TevI (DE836/840) + 2 | This study |
| | glycine residues cloned into the NcoI and BamHI sites (+/−6xHis) | |
| pTevK203G₄-ZFE (or +H) | pACYCryAZf (or +H), with residues 1-K203 of I-TevI (DE837/840) + 4 | This study |
| | glycine residues cloned into the NcoI and BamHI sites (+/−6xHis) | |
| pTevS206-ZFE (or +H) | pACYCryAZf (or +H), with residues 1-S206 of I-TevI (DE838/840) cloned | This study |
| | into the NcoI and BamHI sites (+/−6xHis) | |
| pTevS206G₂-ZFE (or +H) | pACYCryAZf (or +H), with residues 1-S206 of I-TevI (DE839/840) + 2 | This study |
| | glycine residues cloned into the NcoI and BamHI sites (+/−6xHis) | |
| pBmoN221-ZFE (or +H) | pACYCryAZf (or +H), with residues 1-N221 of I-BmoI (DE841/849) | This study |
| | cloned into the NcoI and BamHI sites (+/−6xHis) | |
| pBmoN221G₂-ZFE (or +H) | pACYCryAZf (or +H), with residues 1-N221 of I-BmoI (DE842/849) + 2 | This study |
| | glycine residues cloned into the NcoI and BamHI sites (+/−6xHis) | |
| pBmoN221G₄-ZFE (or +H) | pACYCryAZf (or +H), with residues 1-N221 of I-BmoI (DE843/849) + 4 | This study |
| | glycine residues cloned into the NcoI and BamHI sites (+/−6xHis) | |
| pBmoR223-ZFE (or +H) | pACYCryAZf (or +H), with residues 1-R223 of I-BmoI (DE844/849) | This study |
| | cloned into the NcoI and BamHI sites (+/−6xHis) | |
| pBmoR223G₂-ZFE (or +H) | pACYCryAZf (or +H), with residues 1-R223 of I-BmoI (DE845/849) + 2 | This study |
| | glycine residues cloned into the NcoI and BamHI sites (+/−6xHis) | |
| pBmoR223G₄-ZFE (or +H) | pACYCryAZf (or +H), with residues 1-R223 of I-BmoI (DE846/849) + 4 | This study |
| | glycine residues cloned into the NcoI and BamHI sites (+/−6xHis) | |
| pBmoI226-ZFE (or +H) | pACYCryAZf (or +H), with residues 1-I226 of I-BmoI (DE847/849) cloned | This study |
| | into the NcoI and BamHI sites (+/−6xHis) | |
| pBmoI226G₂-ZFE (or +H) | pACYCryAZf (or +H), with residues 1-I226 of I-BmoI (DE848/849) + 2 | This study |
| | glycine residues cloned into the NcoI and BamHI sites (+/−6xHis) | |
| pTevN201R27A | Similar to pTevN201-ZFE, with an R27A mutation | This study |
| pTevN201G₂R27A | Similar to pTevN201G₂-ZFE, with an R27A mutation | This study |
| pTevN201G₄R27A | Similar to pTevN201G₄-ZFE, with an R27A mutation | This study |
| pTevK203R27A | Similar to pTevK203-ZFE, with an R27A mutation | This study |
| pTevK203G₂R27A | Similar to pTevK203G₂-ZFE, with an R27A mutation | This study |
| pTevK203G₄R27A | Similar to pTevK203G₄-ZFE, with an R27A mutation | This study |
| pTevS206R27A | Similar to pTevS206-ZFE, with an R27A mutation | This study |
| pTevS206G₂R27A | Similar to pTevS206G₂-ZFE, with an R27A mutation | This study |
| pToxTZ1.35 | p11-lacY-wtx1, that contains a 44-bp hybrid I-TevI/ryA zinc-finger homing | This study |
| | site (td bases −27 to +6 fused to the 9-bp ryAZf site) cloned into the | |
| | XbaI and SphI sites (DE824/825) | |
| pToxBZ1.35 | p11-lacY-wtx1, that contains a 44-bp hybrid I-BmoI/ryA zinc-finger homing | This study |
| | site (thyA bases −6 to +27 fused to the 9-bp ryAZf site) cloned into the XbaI | |
| | and SphI sites (DE826/827) | |
| pSP-TZHS1.35 | pSP72, that contains a 44-bp hybrid I-TevI/ryA zinc-finger homing site (td | This study |
| | bases −27 to +6 fused to the 9-bp ryAZf site) cloned into the XbaI and SphI | |
| | sites (DE824/825) | |
| pTZHS1.35 | LITMUS28i, with the 44-bp hybrid I-TevI/ryA zinc-finger homing site (td | This study |
| | bases −27 to +6 fused to the 9-bp ryAZf site) sub-cloned from pSP- | |
| | TZHS1.35 into the BamHI and XhoI sites | |
| pBZHS1.35 | pSP72, that contains a 44-bp hybrid I-Bmol/ryZ zinc-finger homing site | This study |
| | (thyA bases +6 to −27 fused to the 9-bp ryAZf site) cloned into the XbaI and | |
| | SphI sites (DE826/827) | |
| pTZHS2.35 | Similar to pTZHS1.35, with a second Tev-ZFE1.35 target site sub-cloned | This study |
| | from pSP-TZHS1.35 (using PvuII/HpaI) into the SwaI site | |
| pTZHS3.35 | Similar to pTZHS2.35, with the second Tev-ZFE1.35 target site in the | This study |
| | alternate orientation | |
| pToxTZ1.35G5A | Similar to pToxTZ1.35, with a G-23A substitution (DE917/918) | This study |
| pToxTZ1.35G5A/C1A | Similar to pToxTZ1.35, with G-23A and C1A substitutions (DE919/920) | This study |
| pTZHS1.35G5A | Similar to pTZHS1.35, with a G5A substitution | This study |
| pTZHS1.35G5A/C1A | Similar to pTZHS1.35, with G5A and C1A substitution | This study |
| pToxTZ1.34 | p11-lacY-wtx1, that contains a 43-bp hybrid I-TevI/ryA zinc-finger homing | This study |
| | site (td bases −27 to +5 fused to the 9-bp ryAZf site) cloned into the XbaI | |
| | and SphI sites | |
| pToxTZ1.33 | p11-lacY-wtx1, that contains a 42-bp hybrid I-TevI/ryA zinc-finger homing | This study |
| | site (td bases −27 to +4 fused to the 9-bp ryAZf site) cloned into the XbaI | |
| | and SphI sites | |
| pToxBZ1.34 | p11-lacY-wtx1, that contains a 43-bp hybrid I-BmoI/ryA zinc-finger | This study |
| | homing site (thyA bases −6 to +26 fused to the 9-bp ryAZf site) cloned into | |
| | the XbaI and SphI sites (DE826/827) | |
| pToxBZ1.33 | p11-lacY-wtx1, that contains a 42-bp hybrid I-BmoI/ryA zinc-finger homing | This study |
| | site (thyA bases −6 to +25 fused to the 9-bp ryAZf site) cloned into the XbaI | |
| | and SphI sites (DE826/827) | |
| pTZHS1.34 | Similar to pTZHS1.35, with a 43-bp hybrid I-TevI/ryA zinc-finger homing | This study |
| | site (td bases −27 to +5 fused to the 9-bp ryAZf site) | |
| pTZHS1.33 | Similar to pTZHS1.35, with a 42-bp hybrid I-TevI/ryA zinc-finger homing | This study |
| | site (td bases −27 to +4 fused to the 9-bp ryAZf site) | |
| pBZHS1.34 | Similar to pBZHS1.35, with a 43-bp hybrid I-BmoI/ryA zinc-finger homing | This study |
| | site (thyA bases +6 to −26 fused to the 9-bp ryAZf site) | |
| pBZHS1.33 | Similar to pBZHS1.35, with a 42-bp hybrid I-BmoI/ryA zinc-finger homing | This study |
| | site (thyA bases +6 to −25 fused to the 9-bp ryAZf site) | |
| pTZHS2.34 | Similar to pTZHS2.35, with both Tev-ZFE target sites as 43-bp hybrid I- | This study |
| | TevI/ryA zinc-finger homing site (td bases −27 to +5 fused to the 9-bp ryAZf | |
| | site) | |
| pTZHS3.34 | Similar to pTZHS3.35, with both Tev-ZFE target sites as 43-bp hybrid I- | This study |
| | TevI/ryA zinc-finger homing site (td bases −27 to +5 fused to the 9-bp ryAZf | |
| | site) | |
| pTZHS2.33 | Similar to pTZHS2.35, with both Tev-ZFE target sites as 42-bp hybrid I- | This study |
| | TevI/ryA zinc-finger homing site (td bases −27 to +4 fused to the 9-bp ryAZf | |
| | site) | |
| pTZHS3.33 | Similar to pTZHS3.35, with both Tev-ZFE target sites as 42-bp hybrid I- | This study |
| | TevI/ryA zinc-finger homing site (td bases −27 to +4 fused to the 9-bp ryAZf | |
| | site) | |
| pToxTZ1.34G5A | Similar to pToxTZ1.34, with a G5A substitution | This study |
| pToxTZ1.34G5A/C1A | Similar to pToxTZ1.34, with G5A and G-27A substitutions | This study |
| pToxTZ1.33G5A | Similar to pToxTZ1.33, with G5A substitution | This study |
| pToxTZ1.33G5A/C1A | Similar to pToxTZ1.33, with G5A and G-27A substitutions | This study |
| pTZHS1.34G5A | Similar to pTZHS1.34, with G5A substitution | This study |
| pTZHS1.33G5A | Similar to pTZHS1.33, with G5A substitution | This study |
| pTZHS1.34G5A/C1A | Similar to pTZHS1.34, with G5A and C1A substitution | This study |
| pTZHS1.33G5A/C1A | Similar to pTZHS1.33, with G5A and C1A substitution | This study |

1. Chen, Z. and Zhao, H. (2005) A highly sensitive selection method for directed evolution of homing endonucleases. Nucleic Acids Res. 33: e154-

2. Kleinstiver, B. P., Fernandes, A. D., Gloor, G. B. and Edgell, D. R. (2010) A unified genetic, computational and experimental framework identifies functionally relevant residues of the homing endonuclease I-BmoI. Nucleic Acids Res., 38, 2411-2427.

TABLE 2
Oligonucleotides used in this study

| Name | Sequence (5′-3′) | Notes |
|---|---|---|
| DE410 | GGAAGAAGTGGCTGATCTCAGC (SEQ | Forward primer to generate all cycle-seq |
| | ID NO: 15) | products for target sites cloned into |
| | | pTox |
| DE411 | CAGACCGCTTCTGCGTTCTG (SEQ ID | Reverse primer to generate all cycle-seq |
| | NO: 16) | products for target sites cloned into |
| | | pTox |
| DE613 | GCTAAAGATTTTGAAAAGGCATGGA | Forward quikchange primer to create |
| | AGAAGCATTTTAAAG (SEQ ID NO: 17) | R27A Tev-ZFEs |
| DE614 | CTTTAAAATGCTTCTTCCATGCCTTTT | Reverse quikchange primer to create |
| | CAAAATCTTTAGC (SEQ ID NO: 18) | R27A Tev-ZFEs |
| DE824 | CTAGACAACGCTCAGTAGATGTTTTC | Top-strand oligo to clone the hybrid 35- |
| | TTGGGTCTACCGTTTCCCACGCCGCA | bp I-TevI/9-bp ryAZf using XbaI and |
| | TG (SEQ ID NO: 19) | SphI |
| DE825 | CGGCGTGGGAAACGGTAGACCCAAG | Bottom-strand oligo to clone the hybrid |
| | AAAACATCTACTGAGCGTTGT (SEQ | 35-bp I-TevI/9-bp ryAZf using XbaI and |
| | ID NO: 20) | SphI |
| DE826 | CTAGAGCCCGTAGTAATGACATGGCC | Top-strand oligo to clone the hybrid 35- |
| | TTGGGAAATCCCTTTCCCACGCCGCA | bp I-BmoI/9-bp ryAZf using XbaI and |
| | TG (SEQ ID NO: 21) | SphI |
| DE827 | CGGCGTGGGAAAGGGATTTCCCAAG | Bottom-strand oligo to clone the hybrid |
| | GCCATGTCATTACTACGGGCT (SEQ | 35-bp I-BmoI/9-bp ryAZf using XbaI and |
| | ID NO: 22) | SphI |
| DE832 | CCGCGGATCCATTACTAGGCTTTTTA | Reverse primer for TevN201-ZFE |
| | CC (SEQ ID NO: 23) | cloning, BamHI site underlined |
| DE833 | CCGCGGATCCACCACCATTACTAGGC | Reverse primer for TevN201G₂-ZFE |
| | TTTTTACC (SEQ ID NO: 24) | cloning, BamHI site underlined |
| DE834 | CCGCGGATCCACCACCACCACCATTA | Reverse primer for TevN201G₄-ZFE |
| | CTAGGCTTTTTACC (SEQ ID NO: 25) | cloning, BamHI site underlined |
| DE835 | CCGCGGATCCTTTAATATTACTAGGC | Reverse primer for TevK203-ZFE |
| | TTTTAC (SEQ ID NO: 26) | cloning, BamHI site underlined |
| DE836 | CCGCGGATCCACCACCTTTAATATTA | Reverse primer for TevK203G₂-ZFE |
| | CTAGGCTTTTTAC (SEQ ID NO: 26) | cloning, BamHI site underlined |
| DE837 | CCGCGGATCCACCACCACCACCTTTA | Reverse primer for TevK203G₄-ZFE |
| | ATATTACTAGGCTTTTTAC (SEQ ID | cloning, BamHI site underlined |
| | NO: 28) | |
| DE838 | CCGCGGATCCTGAAATCTTTTTAATA | Reverse primer for TevS206-ZFE |
| | TTACTAGGC (SEQ ID NO: 29) | cloning, BamHI site underlined |
| DE839 | CCGCGGATCCACCACCTGAAATCTTT | Reverse primer for TevS206G₂-ZFE |
| | TTAATATTACTAGGC (SEQ ID NO: 30) | cloning, BamHI site underlined |
| DE840 | GCCGCCATGGGTAAAAGCGGAATTT | Forward primer for Tev-ZFE cloning, |
| | ATCAGATT (SEQ ID NO: 31) | NcoI site underlined |
| DE841 | CCGCGGATCCGTTTTTCGGTTTACGA | Reverse primer for BmoN221-ZFE |
| | CC (SEQ ID NO: 32) | cloning, BamHI site underlined |
| DE842 | CCGCGGATCCACCACCGTTTTTCGGT | Reverse primer for BmoN221G₂-ZFE |
| | TTACGACC (SEQ ID NO: 33) | cloning, BamHI site underlined |
| DE843 | CCGCGGATCCACCACCACCACCGTTT | Reverse primer for BmoN221G₄-ZFE |
| | TTCGGTTTACGACC (SEQ ID NO: 34) | cloning, BamHI site underlined |
| DE844 | CCGCGGATCCACGAGAGTTTTTCGGT | Reverse primer for BmoR223-ZFE |
| | TTACG (SEQ ID NO: 35) | cloning, BamHI site underlined |
| DE845 | CCGCGGATCCACCACCACGAGAGTTT | Reverse primer for BmoR223G₂-ZFE |
| | TTCGGTTTACG (SEQ ID NO: 36) | cloning, BamHI site underlined |
| DE846 | CCGCGGATCCACCACCACCACCACG | Reverse primer for BmoR223G₄-ZFE |
| | AGAGTTTTTCGGTTTACG (SEQ ID | cloning, BamHI site underlined |
| | NO: 37) | |
| DE847 | CCGCGGATCCGATAACCGGACGAGA | Reverse primer for BmoI26-ZFE |
| | GTTTTTCGG (SEQ ID NO: 38) | cloning, BamHI site underlined |
| DE848 | CCGCGGATCCACCACCGATAACCGG | Reverse primer for BmoI226G₂-ZFE |
| | ACGAGAGTTTTTCGG (SEQ ID NO: 39) | cloning, BamHI site underlined |
| DE849 | GCCGCCATGGGTAAATCTGGTGTTTA | Forward primer for Bmo-ZFE cloning, |
| | CAAAATC (SEQ ID NO: 40) | NcoI site underlined |
| DE850 | CTTGGGTCTACCGTTCCCACGCCGCA | Forward quikchange primer to make the |
| | TG (SEQ ID NO: 41) | 1.34 I-TevI/ryA zinc-finger target site |
| DE851 | CATGCGGCGTGGGAACGGTAGACCC | Reverse quikchange primer to make the |
| | AAG (SEQ ID NO: 42) | 1.34 I-TevI/ryA zinc-finger target site |
| DE852 | CTTGGGTCTACCGTCCCACGCCGCAT | Forward quikchange primer to make the |
| | G (SEQ ID NO: 43) | 1.33 I-TevI/ryA zinc-finger target site |
| DE853 | CATGCGGCGTGGGACGGTAGACCCA | Reversequikchange primer to make the |
| | AG (SEQ ID NO: 44) | 1.33 I-TevI/ryA zinc-finger target site |
| DE854 | GCCTTGGGAAATCCCTTCCCACGCCG | Forward quikchange primer to make the |
| | CATG (SEQ ID NO: 45) | 1.34 I-BmoI/ryA zinc-finger target site |
| DE855 | CATGCGGCGTGGGAAGGGATTTCCCA | Reverse quikchange primer to make the |
| | AGGC (SEQ ID NO: 46) | 1.34 I-BmoI/ryA zinc-finger target site |
| DE856 | GCCTTGGGAAATCCCTCCCACGCCGC | Forward quikchange primer to make the |
| | ATG (SEQ ID NO: 47) | 1.33 I-BmoI/ryA zinc-finger target site |
| DE857 | CATGCGGCGTGGGAGGGATTTCCCAA | Reverse quikchange primer to make the |
| | GGC (SEQ ID NO: 48) | 1.33 I-BmoI/ryA zinc-finger target site |
| DE858 | CAGAAACAGCTGGTTTAATAACATCA | Forward quikchange primer to add stops |
| | TCACCACTAACTCG (SEQ ID NO: 49) | to the 3′-end of the ryA zinc-finger |
| DE859 | CGAGTTAGTGGTGATGATGTTATTAA | Reverse quikchange primer to add stops |
| | ACCAGCTGTTTCTG (SEQ ID NO: 50) | to the 3′-end of the ryA zinc-finger |
| DE917 | CTAGACAACACTCAGTAGATGTTTTC | Top strand oligo similar to DE824 with |
| | TTGGGTCTACCGTTTCCCACGCCGCA | G5A substitution |
| | TG (SEQ ID NO: 51) | |
| DE918 | CGGCGTGGGAAACGGTAGACCCAAG | Bottom strand oligo similar to DE825 |
| | AAAACATCTACTGAGTGTTGT (SEQ | withe C1T substitution |
| | ID NO: 52) | |
| DE919 | CTAGAAAACACTCAGTAGATGTTTTC | Top strand oligo similar to DE824 with |
| | TTGGGTCTACCGTTTTCCCACGCCGCA | G5A and C1A substitutions |
| | TG (SEQ ID NO: 53) | |
| DE920 | CGGCGTGGGAAACGGTAGACCCAAG | Bottom strand oligo similar to DE825 |
| | AAAACATCTACTGAGTGTTTT (SEQ | with C1T and G5T substitutions |
| | ID NO: 54) | |

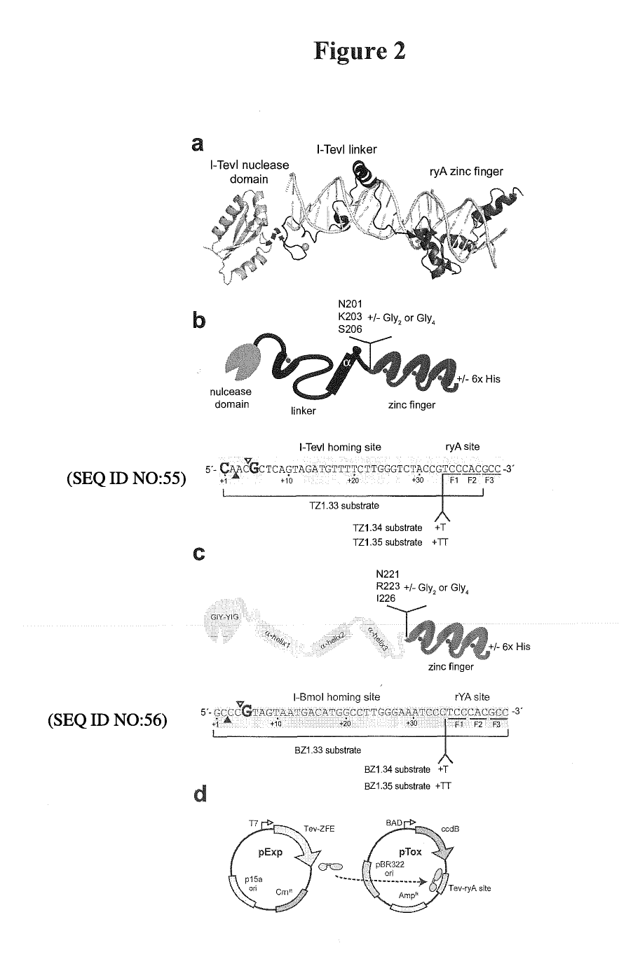

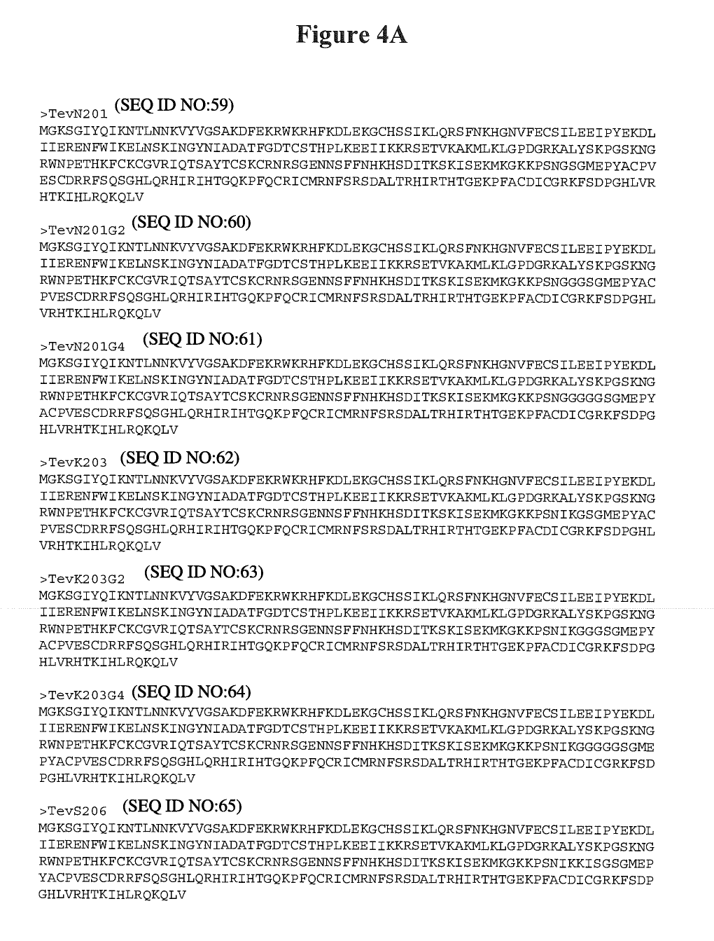

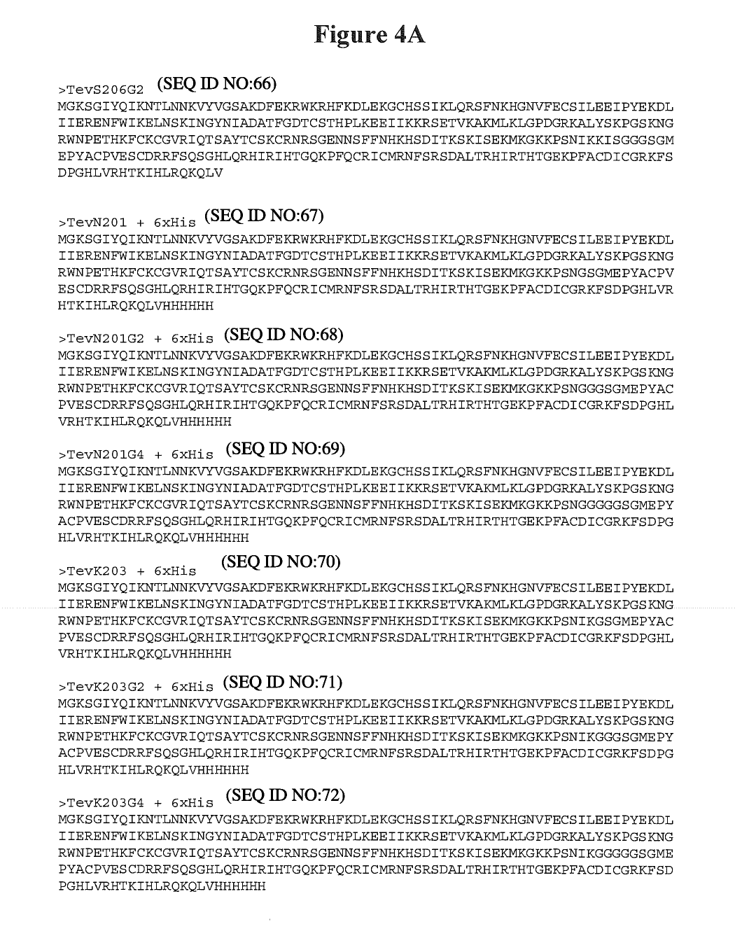

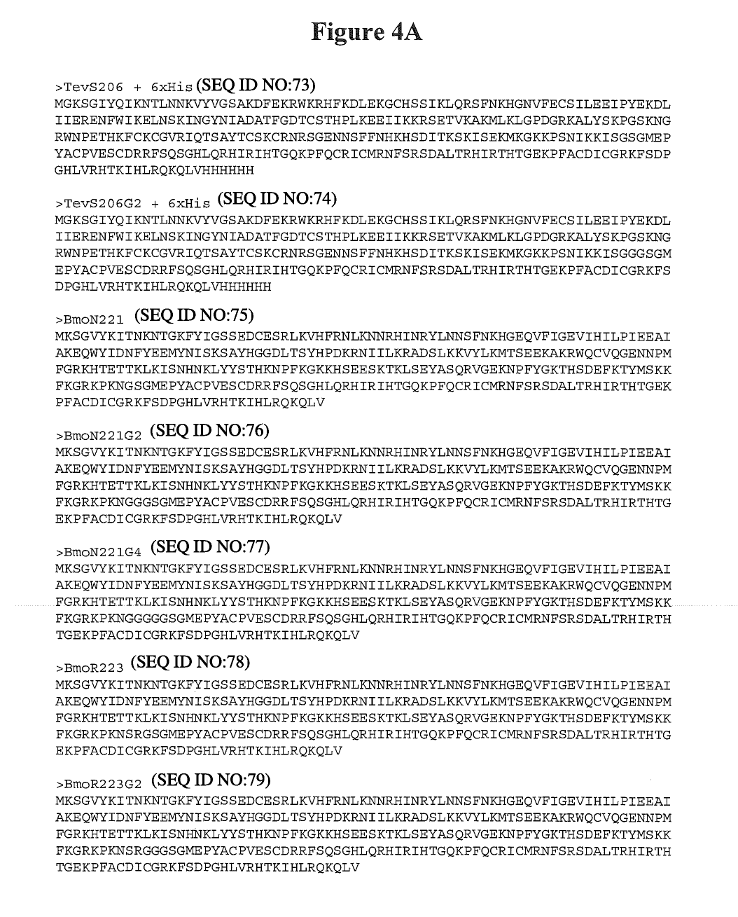

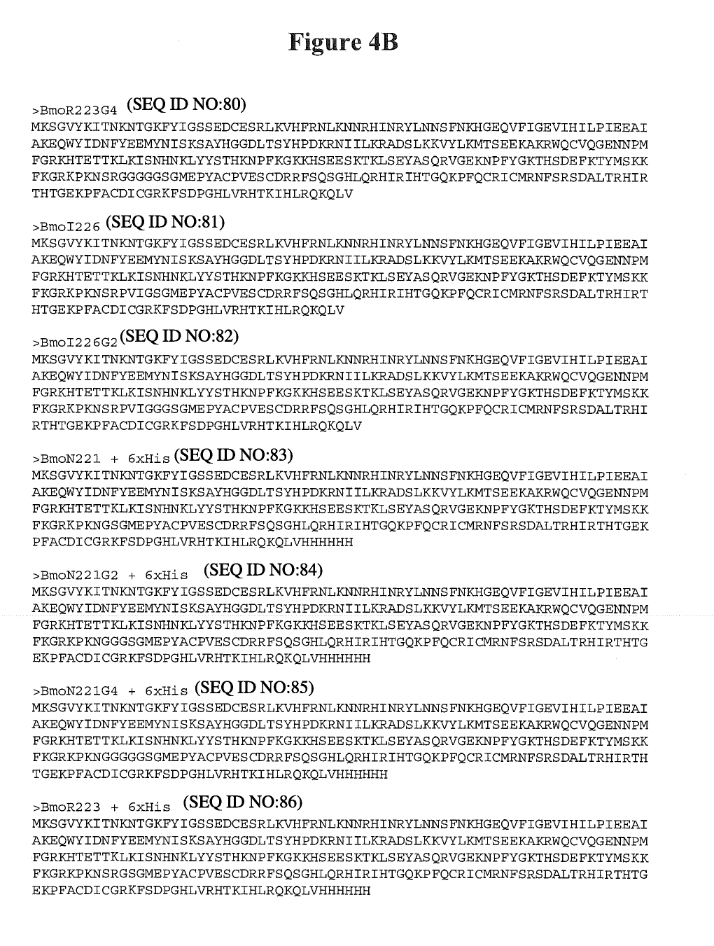

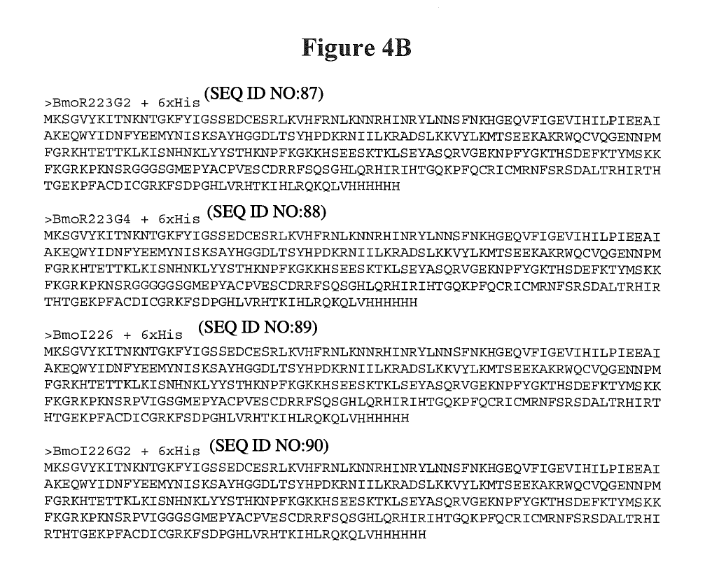

The ryA zinc-finger gene was synthesized by Integrated DNA Technologies with 5′-BamHI and 3′-XhoI sites and a C-terminal 6-histidine tag and cloned into pACYCDuet-1 to generate pACYCryAZf+H. A stop codon was introduced at the 3′ end of the ryAZf gene using Quikchange (Stratagene) to generate pACYCryAZf. The I-TevI and I-BmoI GIY-YIG domains were PCR amplified from bacteriophage T4 gDNA and pACYCIBmoI, respectively, and cloned into pACYCryAXf+H and pACYCryAZf. The R27A mutants of Tev-ZFEs were generated using Quickchange mutagenesis (DE613/614). The sequences of all GIY-ZFEs constructed are listed in FIG. 4). The hybrid target sites (FIGS. 2B and 2C) were cloned into the toxic reporter plasmid p11-lacY-wtx1 to generate pToxTZ1.35 and pToxBZ1.35. Identical Tev-ryA and Bmo-ryA target sites were generated in pSP72 for in vitro cleavage assays. The Tev-ryA site hybrid homing site were also cloned into LITMUS28i using BamHI and XhoI to generate pTZHS1.35. The two-site Tev-ZF plasmids were created by sub-cloning the PvuII/HpaI fragment from pSP-TZHS1.35 into the SwaI site of pTZHS1.35 to generate pTZHS2.35 and pTZHS3.35 (with the second TZHS in either orientation). The G5A or C1A/G5A mutations were introduced into pToxTZ and pTZHS plasmids by Quickchange mutagenesis. All constructs were verified by sequencing.

### Two-Plasmid Genetic Selection

The two plasmid genetic selection was performed as described with toxic (reporter) plasmids containing hybrid Tev- or Bmo-ryA target sites, or mutant ryA target sites (with G5A or C1A/G5A substitutions), or plasmids lacking a target site (p11-lacY-wtx1). Survival percentage was calculated by dividing the number of colonies observed on selective by those observed on non-selective plates.

### Protein Purification

Cultures overexpressing either TevN201-ZFE or BmoN221-ZFE were grown at 37° C. to an OD₆₀₀˜0.5 and expression induced by 0.5 mM IPTG (Bio Basic Inc.) overnight at 15° C. Cells were harvested by centrifugation at 8983×g for 12 minutes, re-suspended in binding buffer (20 mM Tris-HCl pH 8.0, 500 mM NaCl, 10 mM imidazole, 5% glycerol, and 1 mM DDT), and lysed by French press. The cell lysate was clarified by centrifugation at 20400×g, followed by sonication for 30 seconds, and centrifugation at 20400×g for 15 minutes. The clarified lysate was loaded onto a 1 mL HisTrap-HP column (GE Healthcare), washed with 15 mL binding buffer and then 10 mL wash buffer (20 mM Tris-HCl (pH 8.0), 500 mM NaCl, 50 mM imidazole, 5% glycerol and 1 mM DDT). Bound proteins were elated in 1.5 mL fractions in four 5 mL step elutions with increasing concentrations of imidazole. Fractions containing GIY-ZFEs were dialyzed twice against 1 L dialysis buffer (20 mM Tris-HCl (pH 8.0), 500 mM NaCl, 5% glycerol, and 1 mM DDT) prior to storage at −80° C. I-BmoI was purified as previously described (Kleinstiver et al. (2010) Nucleic Acids Res 38:2411-2427).

### Cleavage Assays

Single time-point cleavage assays to determine the EC_{0.5max} of N201 Tev-ZFE were performed in buffer containing 20 mM Tris-HCl pH 8.0, 100 mM NaCl, 10 mM MgCl₂, 5% glycerol, 1 mM DTI and 10 nM pTZHS1.33. Reactions were incubated for 3 minutes at 37° C., stopped with 5 μl stop solution (100 mM EDTA, 40% glycerol, and bromophenol blue), and electrophoresed on a 1% agarose gel prior to staining with ethiduium bromide and analysis on an AlphaImager™3400 (Alpha Innotech). The EC_{0.5max} was determined by fitting the data to the equation

$${f}_{( [ endo ] )} = \frac{{f}_{max} * {[ endo ]}^{H}}{{EC}_{0.5 max} + {[ endo ]}^{H}}$$

where f_{([endo])} is the fraction of substrate cleaved at concentration of TevN201-ZFE [endo], fₘₐₓ is the maximal fraction cleavage, with 1 being the highest value, and H is the Hill constant that was set to 1. The Initial reaction velocity was determined using supercoiled plasmid substrate with varying concentrations of TevN201-ZFE (0.7 nM to 47 nM) and buffer as above. Aliquots were removed at various times, stopped and analyzed as above. The data for product appearance was fitted to the equation

P=A(1−e^{-k}^{₁}^{t})+k₂t

where P is product (in nM), A is the magnitude of the initial burst, k₁ is the rate constant (s⁻¹) of the initial burst phase and k₂ is the steady state rate constant (s⁻¹). The two-site plasmid cleavage assays were conducted as above, using 10 nM pTZHS2.33 or pTZHS3.33 as substrates, and ˜90 nM purified TevN201-ZFE. The k_{obs} rate constants were calculated from the decay of supercoiled substrate by fitting to the equation

[C]=[C₀]exp(−k₁t)

where [C] is the concentration (nM) of supercoiled plasmid at time t, [C₀] is the initial concentration of supercoiled substrate (nM), and k₁ is the first order rate constant (in s⁻¹). At least 3 independent trials were conducted for each data set.

### Cleavage Mapping

Mapping of cleavage sites was performed as described (Mueller et al. (1995) EMBO J 14 (22):5724-5735). Briefly, primers were individually end-labeled with γ-³²P ATP, and used in PCR reactions with pTox or pSP72 plasmids carrying Tev-ryA or Bmo-ryA target sites to generate strand-specific substrates. The substrates were incubated with purified protein as above, and electrophoresed in 8% denaturing gels alongside sequencing ladders generated by cycle sequencing with the same end-labeled primers (USB Biologicals).

### Results

### GIY-YIG Homing Endonucleases Function as Monomers

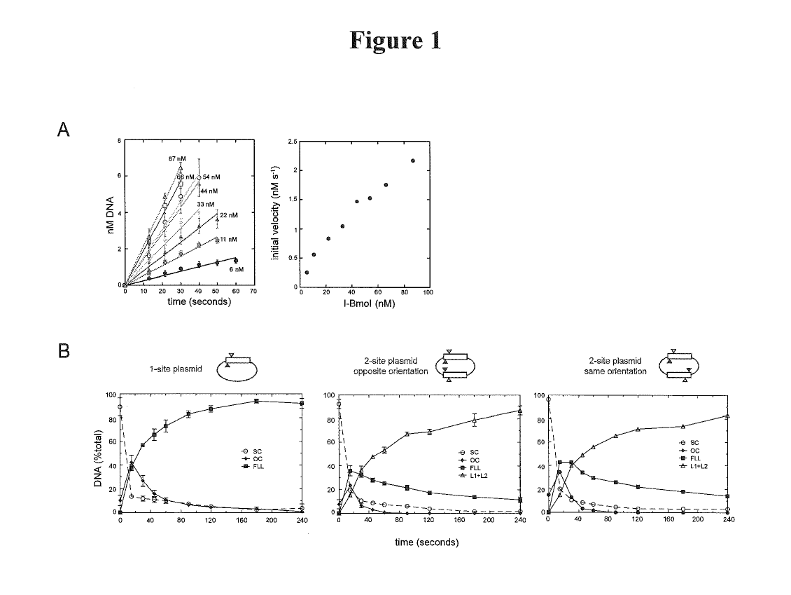

To probe the oligomeric state of GIY-YIG homing endonucleases, it was determined if I-BmoI functions catalytically as a monomer by examining the relationship between protein concentration and initial reaction velocity. This relationship was determined by in vitro cleavage assays using a plasmid substrate with a single thyA target site. As shown in FIG. 1, plotting of the initial reaction velocity versus protein concentration revealed a linear relationship, suggesting that DNA hydrolysis is first order with respect to I-BmoI concentration. This observation was extended by performing cleavage assays with plasmids that contained either one or two copies of the I-BmoI thyA target site under conditions of protein excess (FIG. 1), and a k_{obs(1-site)} of 0.105±0.01 s⁻¹ and a k_{obs(2-site)} of 0.096±0.01 s⁻¹ was calculated. The small differences in the k_{obs} rate constants indicated that I-BmoI does not require two target sites to promote DNA cleavage. In contrast, similar assays with FokI showed a significant rate enhancement for two-site plasmids relative to one-site plasmids, consistent with FokI functioning as a dimer. Cross-linking and gel-filtration studies were also consistent with I-BmoI existing as monomer in solution or when bound to its cognate substrate (FIG. 1). The simplest interpretation of the above data is that the oligomeric status of I-BmoI is not influenced by protein concentration, that cleavage by I-BmoI is non-cooperative, and that I-BmoI functions as a monomer. Furthermore, when considered in the context of past studies showing that the closely related I-TevI binds DNA as a monomer, it is likely that both I-BmoI and I-TevI function as monomers in all steps of DSB formation.

### Construction and Validation of GIY-Zinc Finger Endonucleases

Existing crystal structures were used to model GIY-YIG-zinc finger endonucleases (GIY-ZFEs). For the I-TevI-zinc finger fusions (Tev-ZFE), the Zif268 zinc finger was modeled in place of the H-T-H motif at the C-terminal end of I-TevI (FIGS. 2A and 2B). Actual GIY-ZFEs utilised the ryA zinc finger that targets a sequence in the Drosophila yellow gene. One notable feature of these constructs is the polarity, as the GIY-YIG nuclease domain is fused to the N-terminal end of the ryA protein to mimic the native orientation of the GIY-YIG domain, whereas FokI fusions are to the G-terminal end of zinc-finger proteins. The DNA substrates consisted of 31 to 33 bps of the I-TevI td homing site that is contacted by the linker and nuclease domains, joined to the 9-bp ryA target site (FIG. 2B). In the shortest substrates, the critical G of the 5′-CSSSG-3′ cleavage motif is positioned 28-bp distant from the ryA binding site, in analogy with the native spacing of the I-TevI td homing site. An analogous set of I-BmoI-ryA fusions were constructed (Bmo-ZFEs, FIG. 2C).

The activity of the GIY-ZFEs using a well-described two-plasmid bacterial selection system (FIG. 2D) was determined, where survival is dependent on endonuclease activity, as described in Kleinstiver et al. ((2010). Nucleic Acids Res 38:2411-2427). Eight Tev-ZFEs were tested against three substrates that differed in positioning of the preferred 5′-CXXXG-3′ cleavage motif relative to the ryA binding site (FIG. 2B). All Tev-ZFEs exhibited significant survival, with the highest survival observed against plasmid substrates with the shortest distance between the cleavage motif and ryA-binding site (as shown in Table 3 below). In contrast, no survival was observed when the fusions were tested against the toxic plasmid without an appropriate target site (p11lacYwtx1), demonstrating that survival is dependent on a specific ryA-binding site. The catalytic arginine 27 of the I-TevI nuclease domain was also mutated to alanine in all of the Tev-ZFEs, creating TevR27A-ZFEs. None of the TevR27A-ZFEs survived the assay, showing that survival is dependent on the GIY-YIG nuclease activity. Addition of a C-terminal 6x-His tag to any of the Tev-ZFEs had no effect on activity, as all constructs displayed survival rates very similar to the untagged constructs. The Bmo-ZFEs in the genetic selection were also tested. As described below, enzymatic activity was detected in vitro using purified Bmo-ZFEs. Collectively, these results show that two different GIY-YIG nuclease domains and linkers could be fused to the ryA zinc finger to create chimeric site-specific nucleases.

TABLE 3
Survival of GIY-ZFEs in the two-plasmid genetic selection.

| | Toxic plasmid | | | | | | | | | |
|---|---|---|---|---|---|---|---|---|---|---|
| | pToxTZ1.33 | | | pToxTZ1.34 | | | pToxTZ1.35 | | | |
| GIY-ZFE | WT | G5A | C1A/G5A | WT | G5A | C1A/G5A | WT | G5A | C1A/G5A | p11lacywtx |
| TevN201 | 86.8 ± 5.9 | 0 | 0 | 59.9 ± 9.5 | 0 | 0.2 ± 0.1 | 49.8 ± 9.8 | 0 | 0 | 0 |
| | (6) | | | (6) | | (3) | (6) | | | |
| TevN201G₂ | 72.7 ± 10.7 | 0 | 0 | 56.9 ± 11.2 | 0 | 0 | 38.6 ± 10.3 | 0 | 0 | 0 |
| | (6) | | | (6) | | | (4) | | | |
| TevN201G₄ | 83.7 ± 15.2 | 0 | 0 | 42.8 ± 12.6 | 0 | 0 | 36.3 ± 7.1 | 0 | 0 | 0 |
| | (4) | | | (6) | | | (4) | | | |
| TevN201R27A | 0 | 0 | 0 | 0 | 0 | 0 | 0 | 0 | 0 | 0 |
| TevK203 | 86.8 ± 7.1 | 0 | 0 | 50.7 ± 9.5 | 0 | 0 | 51.0 ± 6.6 | 0 | 0 | 0 |
| | (6) | | | (6) | | | (5) | | | |
| TevK203G₂ | 88 ± 13.9 | 0 | 0 | 53.7 ± 10.4 | 0 | 0 | 46.5 ± 10.9 | 0 | 0 | 0 |
| | (6) | | | (6) | | | (5) | | | |
| TevK203G₄ | 80.7 ± 7.9 | 0.2 ± 0.2 | 0.4 ± 0.3 | 43.6 ± 13.0 | 0 | 0 | 48.0 ± 6.1 | 0 | 0 | 0 |
| | (4) | (3) | (3) | (6) | | | (4) | | | |
| TevK203R27A | 0 | 0 | 0 | 0 | 0 | 0 | 0 | 0 | 0 | 0 |
| TevS206 | 86.6 ± 6.9 | 0 | 0 | 47.1 ± 8.6 | 0 | 0 | 62.3 ± 12.4 | 0 | 0 | 0 |
| | (6) | | | (6) | | | (4) | | | |
| TevS206G₂ | 70.7 ± 8.7 | 0 | 0 | 27.8 ± 7.4 | 0 | 0 | 44.2 ± 16.4 | 0 | 0 | 0 |
| | (4) | | | (6) | | | (4) | | | |
| TevS206R27A | 0 | 0 | 0 | 0 | 0 | 0 | 0 | 0 | 0 | 0 |

¹Fusions are named according to the residue number of I-TevI fused to the N-terminal of the ryA zinc finger (ie. N201 refers to asparagine 201 of I-TevI). G₂ and G₄ refer to a 2- and 4-residue spacer linker, respectively, between the I-TevI and ryA domains. R27A refers to an arginine 27 to alanine mutation.
²Toxic substrate plasmids are designated as described in Materials and Methods.
³Survival percentages are reported as the mean with standard deviation, with the number of replicates in brackets. Selections with zero survival were confirmed by three independent trials.

### GIY-ZFEs Require Specific Sequences for Efficient Cleavage

Both I-TevI and I-BmoI are DNA endonucleases that cleave specific sequences at a defined distance from their primary binding sites. To determine if the chimeric GIY-ZFEs also cleaved substrate in a sequence-specific manner, the TevN201-ZFE and BmoN221-ZFE fusions were purified for in vitro mapping studies (FIGS. 3A and 3B). Using strand-specific end-labeled substrates, the bottom- and top-strand nicking sites of TevN201-ZFE were mapped to lie within the 5′-CXXXG-3′ motif, with ↑ and ↓ representing the bottom- and top-strand nicking sites, respectively (FIG. 3C). The bottom-and top-strand nicking sites of BmoN221-ZFE were mapped to a 5′-XX↑XX↓G-3′ motif, mimicking the native I-BmoI sites. FIG. 3D Thus, both the I-TevI and I-BmoI GIY-YIG nuclease domains cleave DNA specifically in the context of a zinc-finger fusion.

To further demonstrate TevN201-ZFE cleavage specificity, mutations were introduced in the 5′-CXXXG-3′ motif that were previously shown to drastically reduce I-TevI cleavage efficiency (FIG. 3E). Significantly, no survival was observed in the two-plasmid selection assay with pTox plasmids carrying either the single G5A (5′-CXXXA-3′) or double C1A/G5A (5′-AXXXA-3′) substitutions (Table 1), equivalent to mutations at positions C-27 and G-23 of the I-TevI td substrate. Cleavage assays were performed with wild type and mutant substrates and increasing concentrations of TevN201-ZFE to determine the amount of protein required for half-maximal cleavage (EC_{0.5max}). As shown in FIG. 3e˜60 fold and ˜4.7 fold more protein were required to achieve half-maximal cleavage of the double- and single-mutant substrates relative to the wild-type substrate. The greater substrate discrimination observed in the genetic assay likely reflects lower in vivo protein concentrations than those used for in vitro cleavage assays. These results clearly show that the TevN201-ZFE fusion retains the cleavage specificity of the parental I-TevI enzyme and that double nucleotide substitutions can significantly reduce cleavage efficiency. Although the BmoN221-ZFE substrate specificity was not tested extensively, it was shown that the chimeric endonuclease cleaved the Bmo-ryA substrate plasmid, but not the target-less control plasmid.

### GIY-ZFEs Function as Monomers

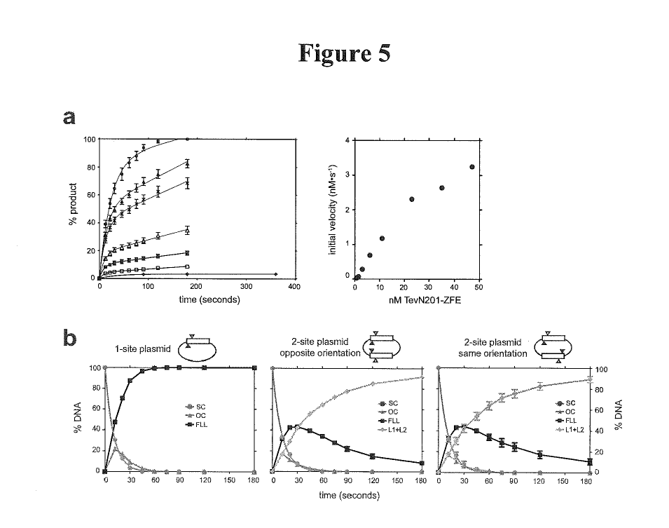

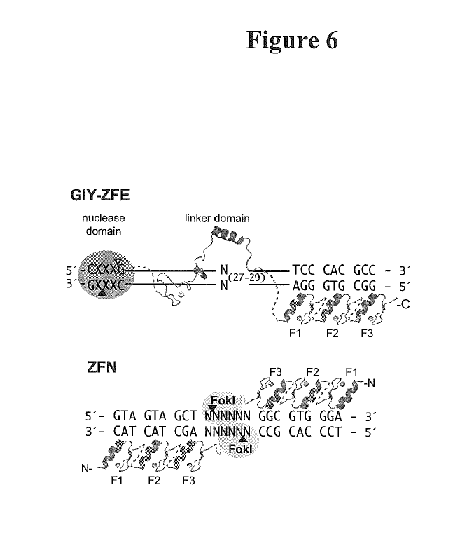

To determine if the GIY-YIG domain retained the ability to function as a monomer in the context of a zinc-finger fusion, cleavage assays were performed to determine the relationship between TevN201-ZFE enzyme concentration and initial reaction velocity. The reaction progress curves indicated an initial burst of cleavage followed by a slower rate of product accumulation (FIG. 5A), consistent with product release being the rate-limiting step. The initial burst phase was used to estimate initial velocity, and plotting against protein concentration yielded a linear relationship (FIG. 5A), suggesting that DNA hydrolysis catalyzed by TevN201-ZFE is first order with respect to protein concentration. Time-course cleavage assays under single-turnover conditions (˜10-fold molar excess of protein to substrate) were also conducted with plasmids that contained one or two Tev-ryA target sites. Two-site plasmids that differed in whether the target sites were in the same or opposite orientations relative to each other were constructed. As shown in FIG. 5B, cleavage of the one-site plasmid yielded k_{obs(1-site)}=0.099±0.001 s⁻¹, and cleavage of the two-site plasmids with target sites in the same or opposite orientations generated very similar rate constants, k_{obs(2-site)}=0.088±0.001 s⁻¹ and 0.089±0.001 s⁻¹, respectively, to the one-site plasmid. Thus, TevN201-ZFE does not require two sites for efficient DNA hydrolysis, consistent with the enzyme functioning as a monomer.

### EXAMPLE 2

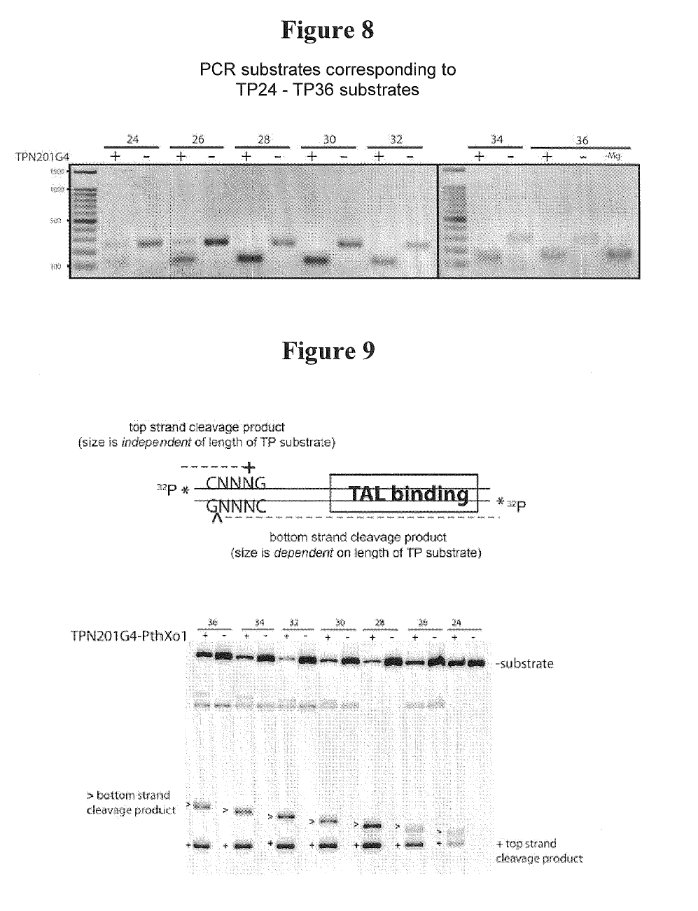

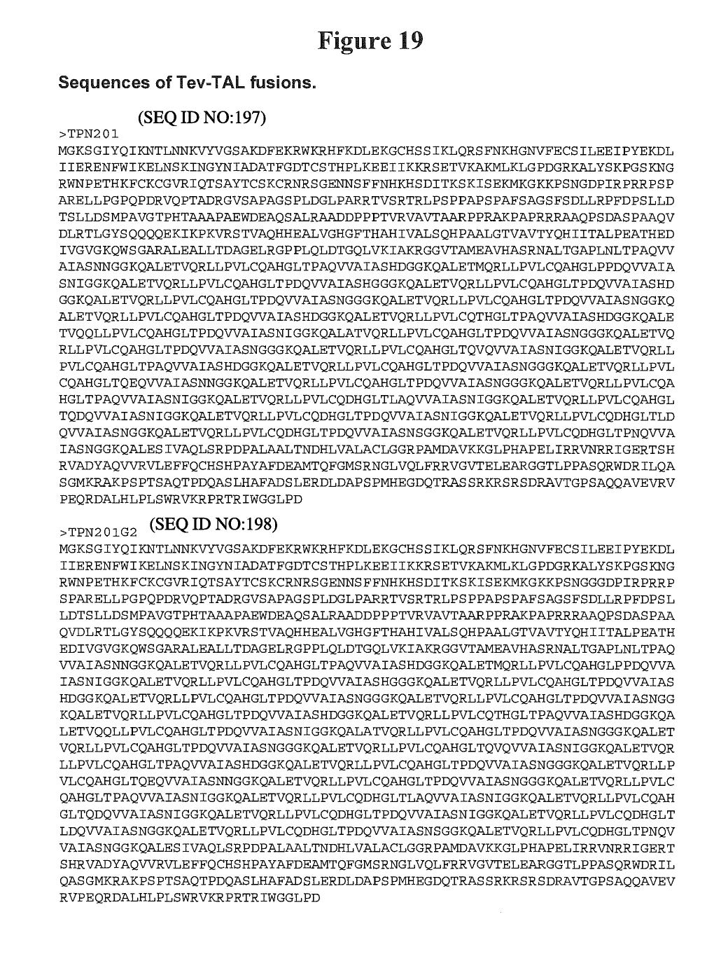

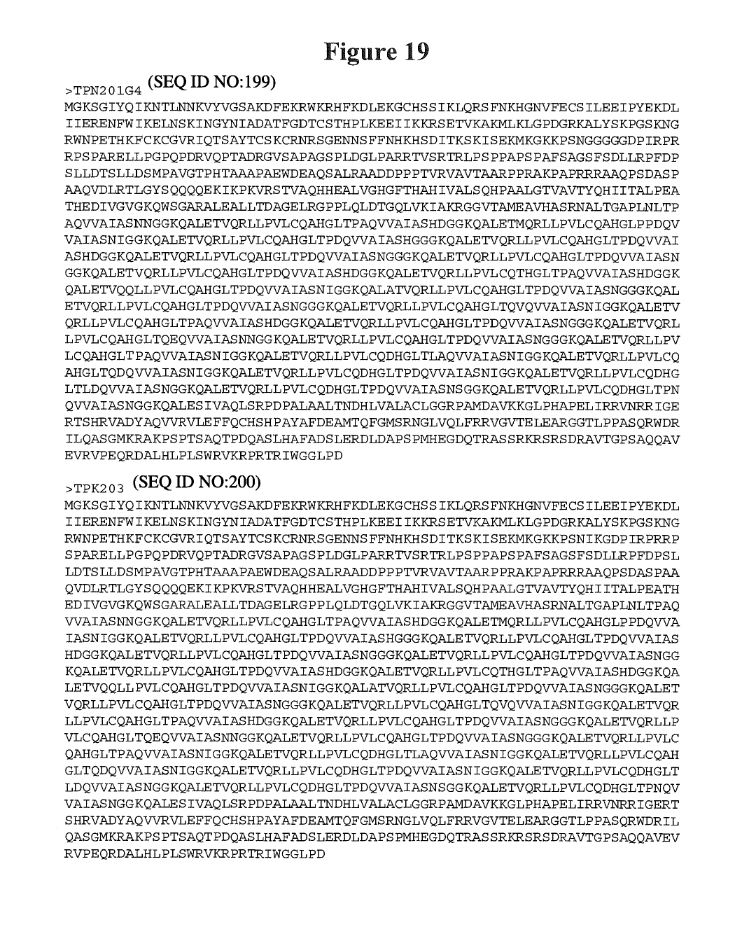

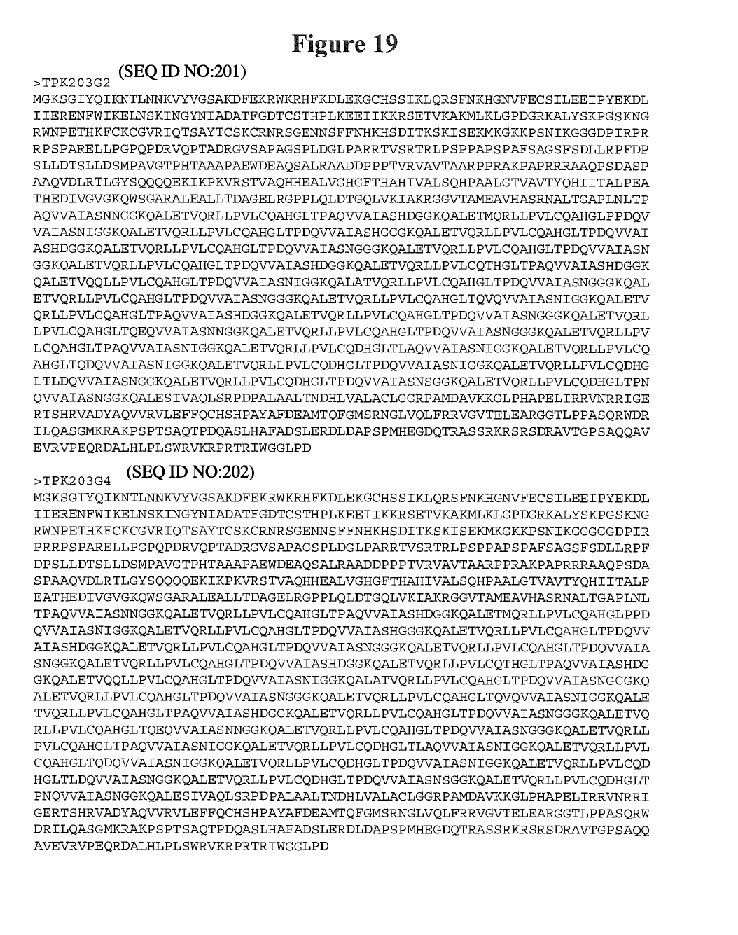

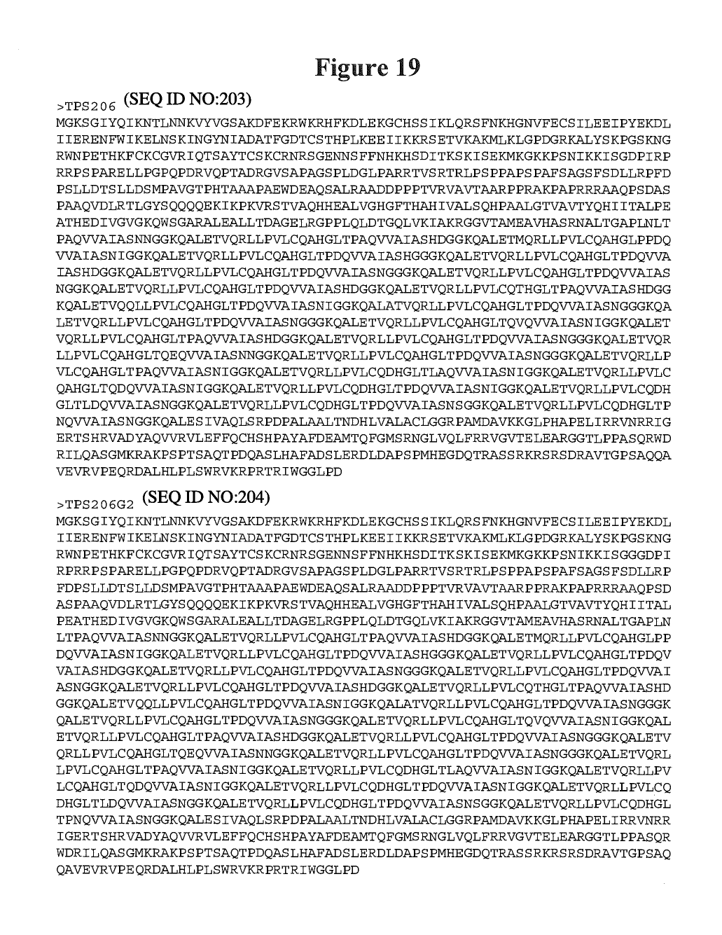

The TevN201(G4)-PthXol TAL-effector fusion (Tev201-TAL, FIG. 7A) was purified from E. coli BL21 (DE3) cells overexposing the fusion that was cloned into pACYC-Duet. The fusion protein was purified un-tagged by ion-exchange chromatography. A number of fusion products were constructed which varied in the size of the I-TevI linking portion that was incorporated. As shown, regions including 201, 203 and 206, with or without additional glycine residues, were made. The full amino acid sequences of fusion products constructed are shown in FIG. 19. The final purification fractions were used for in vitro DNA cleavage assays using either PCR products or radioactively labeled duplex oligonucleotide substrates. As shown in FIG. 8A, the substrates consisted of various lengths of the native I-TevI target sequence derived from the phage T4 td gene that were fused to the 5′ end of the PthXol TAL-effector binding site. The substrates are designated TP (for Tev-PthXol), and number according the length of the I-TevI target site included (TP24 has 24 bp of the I-TevI target site). The substrates were designed as complementary oligonucleotides that were subsequently annealed and cloned into pLitmus. Alternatively, the oligonucleotides were radiolabeled with ³²P, and then annealed. As shown in FIG. 8, when incubated with Tev201-TAL, cleavage was observed on all the PCR products corresponding to the TP24-36 substrates, with varying degrees of efficiency. Divalent metal ion was omitted from one reaction, but cleavage was still observed. This result is consistent with previous data showing that the native I-TevI protein retains activity in the absence of exogenously added divalent metal ion, likely because the nuclease domain has metal bound during purification.

The radioactively labeled DNA substrates were used to map the cleavage sites of the Tev-TAL fusions. The substrates were labeled on both strands, meaning that both the top and bottom strand cleavage products could be mapped. As shown in FIG. 9, two prominent cleavage products were observed with the TP series of substrate when incubated with Tev201-TAL. Note that the site of the bottom strand product varies with the TP substrate tested. The size difference is due to the fact that the position of the bottom strand cleavage site is moved closer to the 3′ end of the duplex DNA substrate (i.e. closer to the TAL binding site) because the shorter TP substrates include less of the native I-TevI site. The top strand cleavage site does not change size, because its position relative to the 5′ end of the duplex substrates does not change in any of the substrates. The sizes of both cleavage products are consistent with specific cleavage by the Tev201-TAL fusion at the CNNNG cleavage motif.

Reference to the amino acid alignment of the linker regions of I-TulaI, I-TevI, and I-BmoI (see FIG. 15D) indicates the regions of conservation and consensus. Indicated is the functionally critical region of the ITevI linker (Kowalski et al. 1999 NAR; Liu et al. 2008, JMB). To one knowledgable in the art, an optimised linker may be generated that includes deletion, replacement, and addition of amino acid sequences using conventional methods. This may include the replacement of the functionally non-critical regions in the linker with other desired sequences.

### EXAMPLE 3

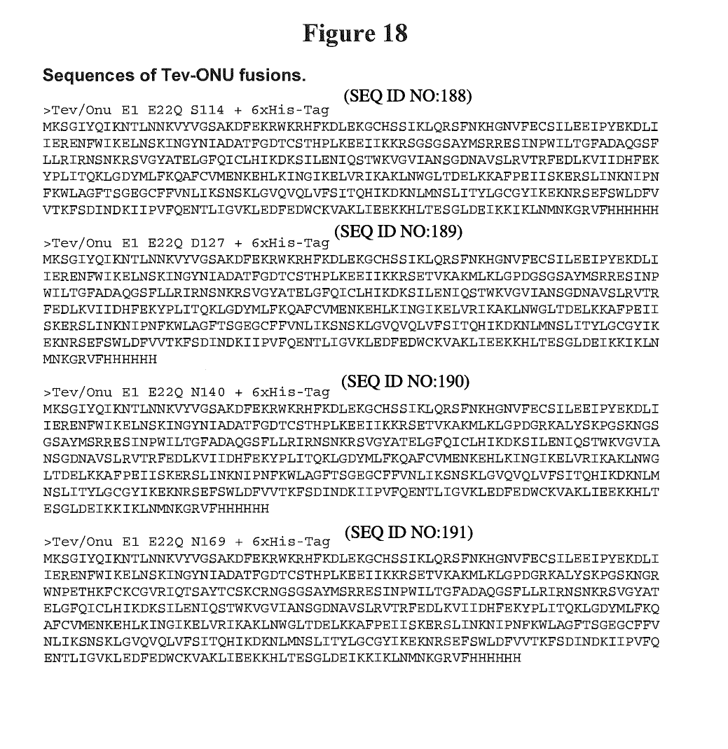

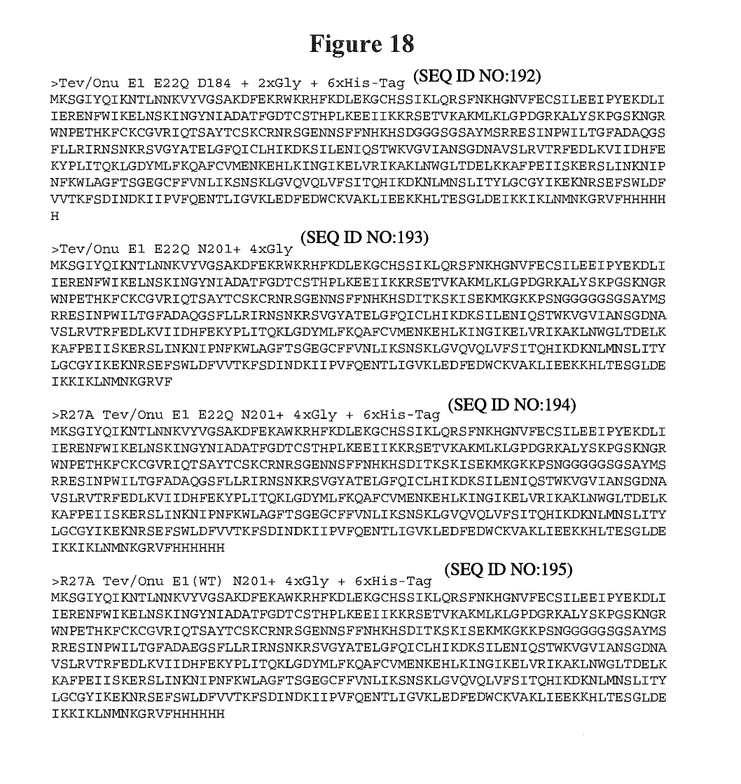

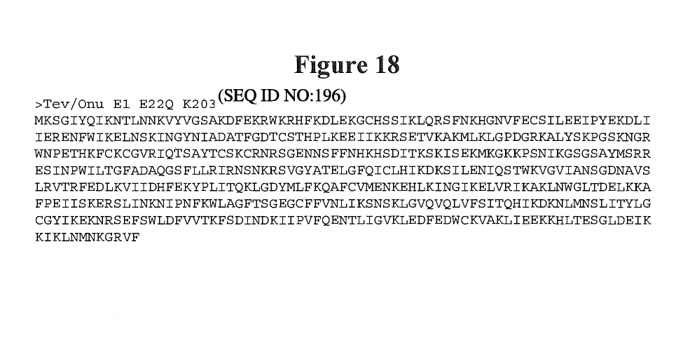

The nucleotide requirements of the I-TevI linker (residues 97-169) for its corresponding region on a substrate was determined. A coupled in vitro/in vivo selection system was used (Edgell et al. Current Biology (2003) 13:973-978) that relies on cleavage of a randomized DNA spacer plasmid library by the Tev169-Onu fusion protein (see FIG. 18 for amino acid sequences of a family of Tev-Onu fusion products that vary in the size of the Tev portion). Cleaved substrates are isolated, and amplified in E. coli, followed by bar-coded PCR for deep-sequencing on an Ion Torrent sequencer.

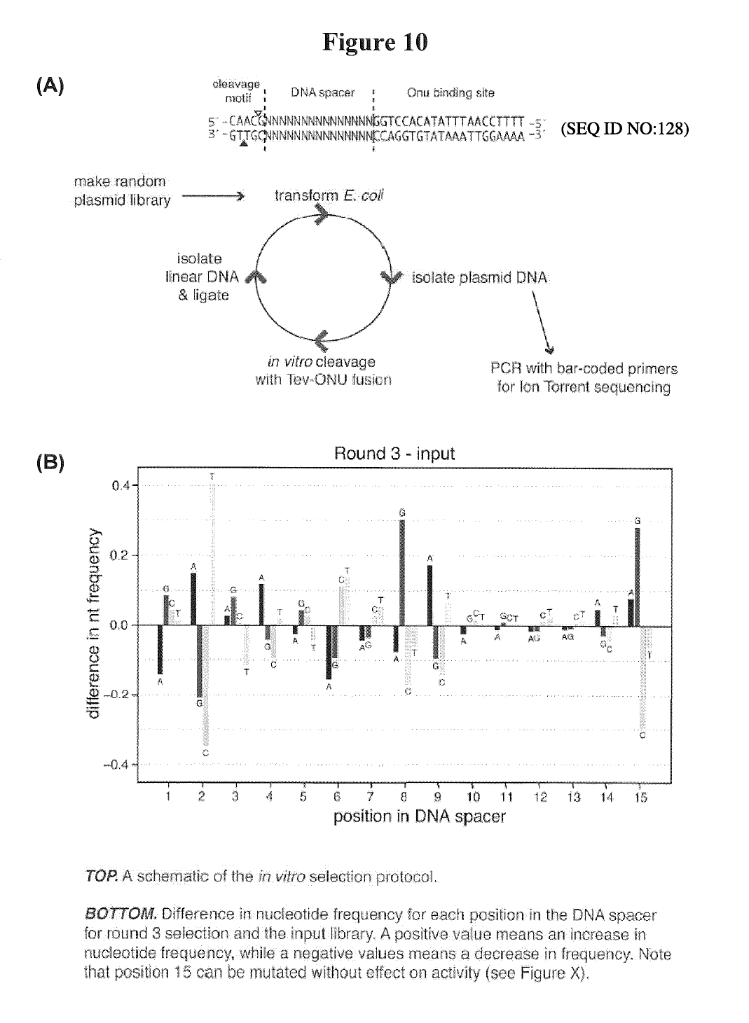

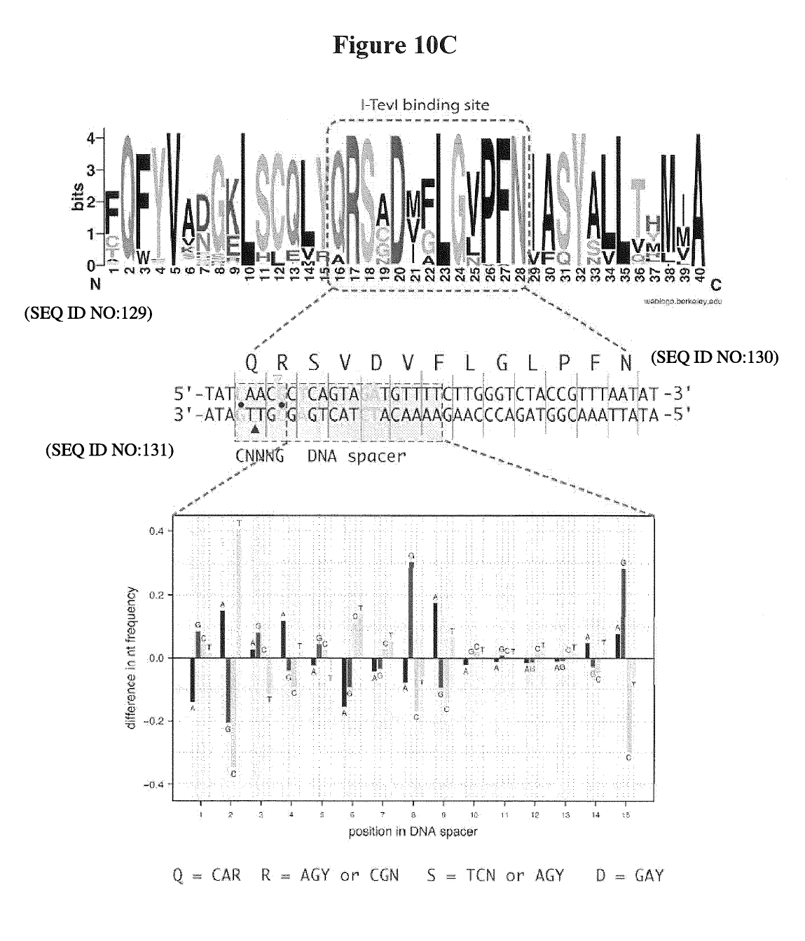

The findings indicate that the I-TevI linker has a nucleotide preference at 3 positions within the DNA spacer, namely, positions 2, 8 and 15 (see FIG. 10a/b). Thus, a consensus DNA sequence for the Tev169 constructs could be 5′ CNNNGN(A/T)NNNNNG(A/T), where N is any nucleotide and the CNNNG is the required cleavage motif. This motif occurs in >93% of all non-redundant human cDNAs at least once (see FIG. 11). FIG. 10c demonstrates the relationship between the nucleotide bias in the DNA spacer region (bottom), and its relationship to the evolutionary conserved amino acids of the I-TevI native target gene thymidylate synthase in bacteriophage T4 (spp). Domain knowledge regarding the original sequence permits refinement of the spacer region identified in FIG. 10b to identify potential artifacts linked to the original sequence bias to generate a viable consensus and indicates the importance of the core spacer sequence comprising CNNGN(A/T), and the sealed optional nature of an additional NNNNNG and the additional terminal (A/T) nucleotide.

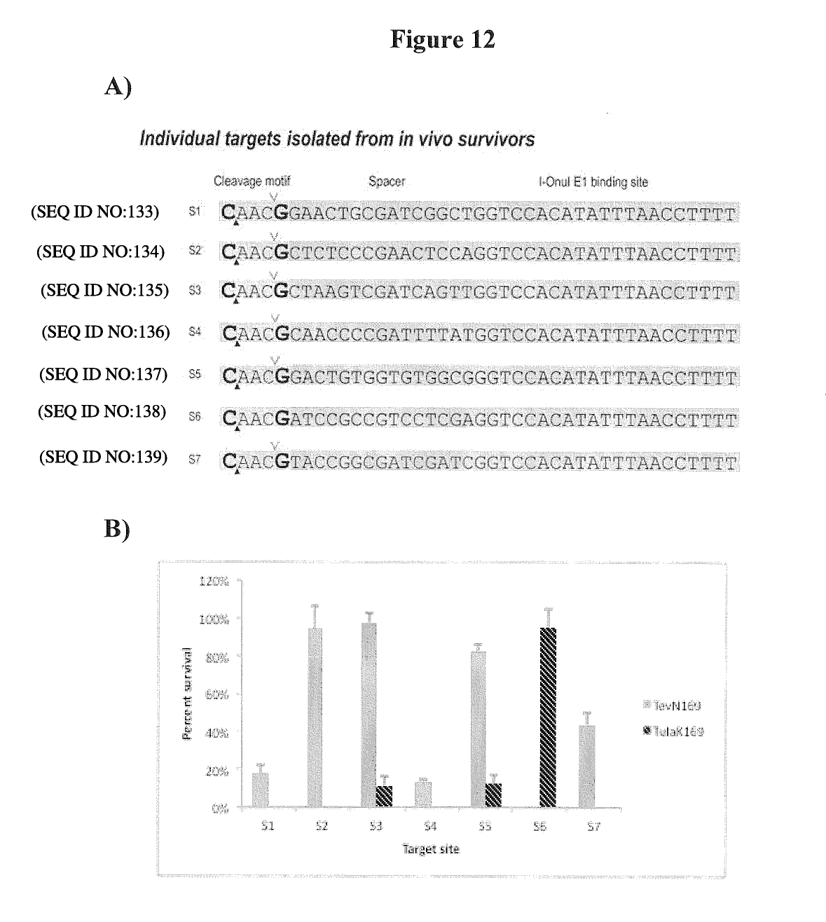

Cleavage efficiency on individual substrates that were selected at random from the DNA spacer library were also tested. This data is shown in FIGS. 12, 13, and 14. FIG. 12 shows the sequences, and the activity of the Tev169-Onu fusion on these sequences in the bacterial two-plasmid assay.

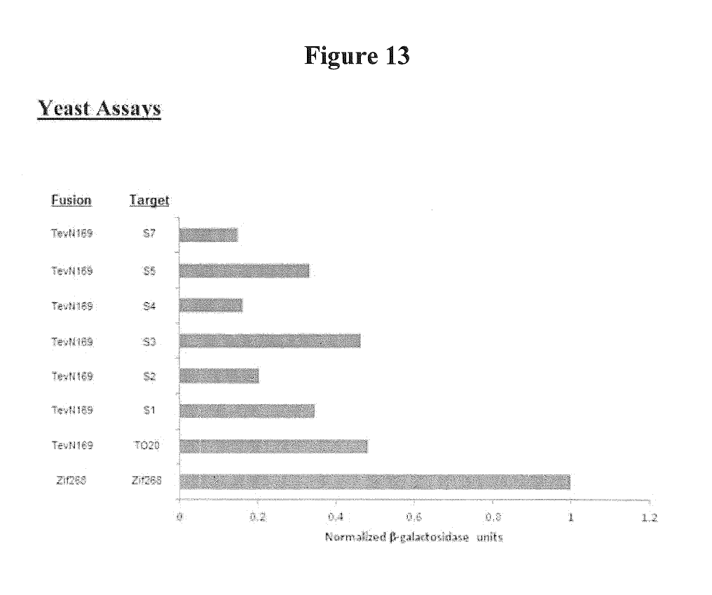

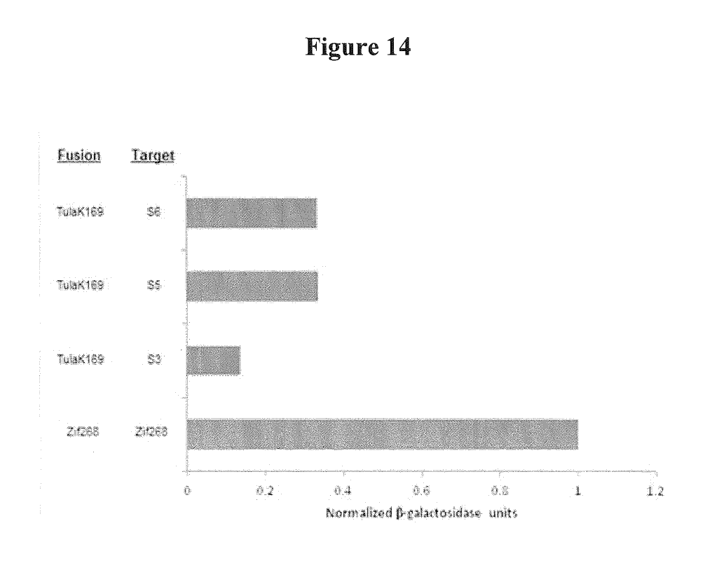

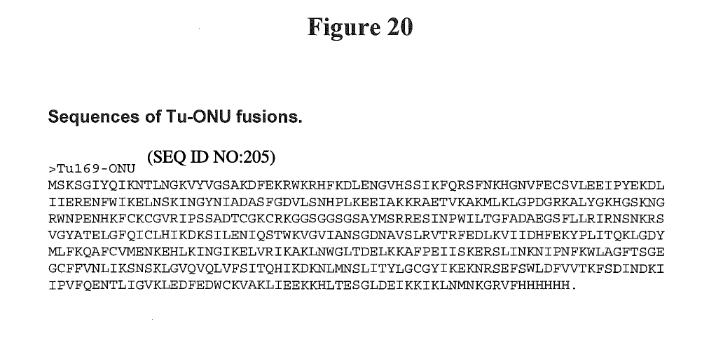

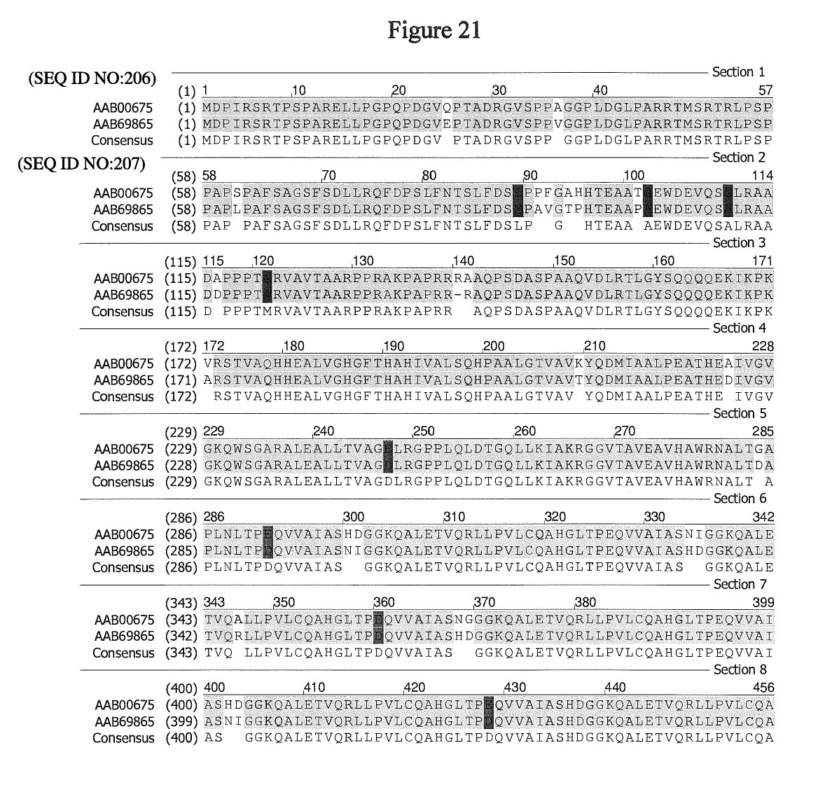

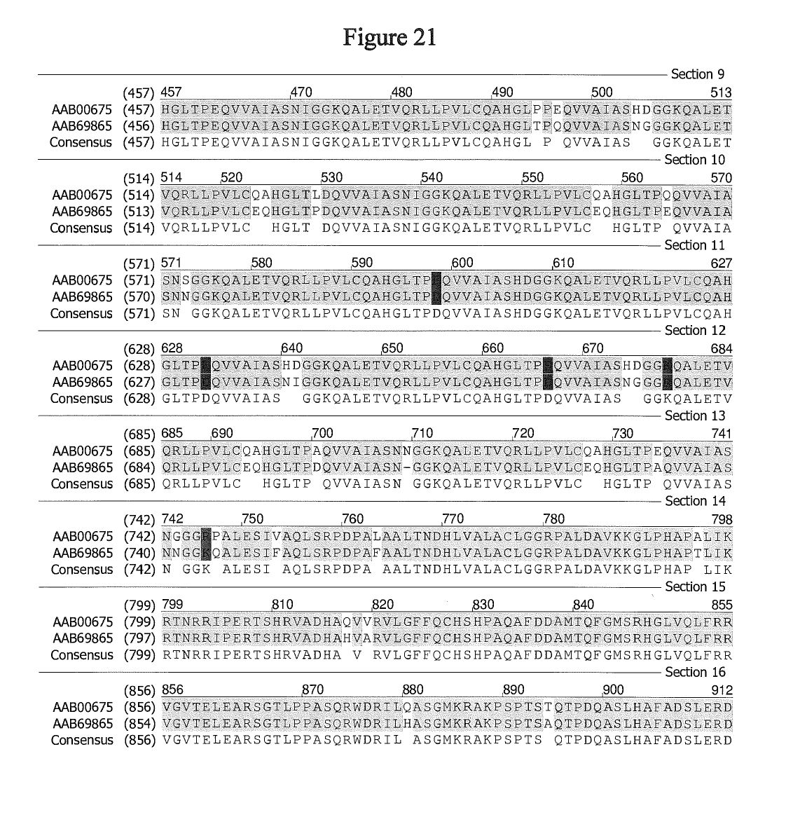

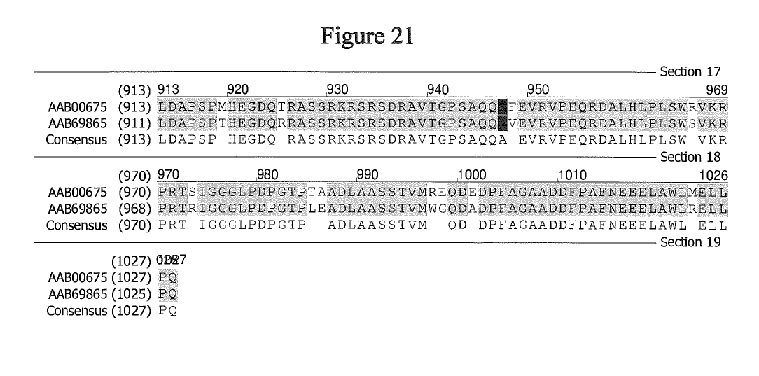

Also included in this analysis is the activity of the Tula-derived fusions (TulaK169, sequence as shown in FIG. 20). FIG. 13 shows the activity of the Tev169-Onu fusions on the substrates in a yeast-based assays, relative to a normalized Zif268 control. FIG. 14 shows the activity of the Tulak169 fusions on a subset of the sequences.

While the foregoing invention has been described in some detail for purposes of clarity and understanding, it will be appreciated by one skilled in the art from a reading of this disclosure that various changes in form and detail can be made without departing from the true scope of the invention and appended claims. All patents and publications cited herein are entirely incorporated herein by reference.

We claim:

1. A chimeric endonuclease comprising a nuclease domain and a DNA-targeting domain, wherein the chimeric endonuclease is capable of cleaving double-stranded DNA as a monomer.

2. A chimeric endonuclease according to claim 1, wherein the nuclease domain is a site specific nuclease domain.

3. A chimeric endonuclease according to claim 2, wherein the nuclease domain is from a homing endonuclease.

4. A chimeric endonuclease according to claim 3, wherein the homing endonuclease is a GIY-YIG homing endonuclease.

5. A chimeric endonuclease according to claim 4, wherein the homing endonuclease is I-TevI.

6. A chimeric endonuclease according to claim 1, further comprising a linking domain.

7. A chimeric endonuclease according to claim 1, wherein the DNA-targeting domain is a TAL domain.

8. A chimeric endonuclease comprising a I-TevI endonuclease domain and a TAL DNA-targeting domain.

9. A chimeric endonuclease according to claim 8, wherein the I-TevI nuclease is N-terminal to the TAL domain.

10. A nucleic acid molecule encoding a chimeric endonuclease according to claim 1.

11. A method of inactivating a gene, comprising:
introducing a nucleic acid molecule encoding a chimeric endonuclease according to claim 1 into a cell comprising the gene under conditions causing the expression of the chimeric endonuclease, wherein the chimeric endonuclease comprises a DNA-targeting domain that binds the and cleaves it.

12. A method according to claim 11, wherein the expression of the chimeric endonuclease is transient.

13. A method according to claim 11, wherein the cell is a plant cell.

14. A method according to claim 11, wherein the nucleic acid molecule is an mRNA.

15. A method of altering a gene in a cell, comprising:
introducing a first nucleic acid molecule encoding a chimeric endonuclease according to claim 1 into a cell comprising the gene under conditions causing the expression of the chimeric endonuclease and cleavage of the gene;
introducing a second nucleic acid molecule into the cell wherein the second nucleic acid molecule comprises a region having a nucleotide sequence that has a high degree of sequence identity to all or a portion of the gene in the region of the cleavage site under conditions causing homologous recombination to occur between the second nucleic acid molecule and the gene.

16. A method according to claim 15, wherein the region comprises 500 basepairs that are homologous to the gene.

17. A method according to claim 16, wherein the region comprises an altered sequence when compared to the gene of interest.

18. A method according to claim 17, wherein the region comprises one or more mutations that will result in changes to one or more amino acids in a protein encoded by the gene.

19. A method according to claim 18, wherein the chimeric endonuclease is transiently expressed in the cell.

20. A method according to claim 19, wherein the first nucleic acid molecule is mRNA.

21. A method according to claim 15, wherein the second nucleic acid molecule is a linear DNA molecule.

22. A method according to claim 15, wherein the cell is a plant cell.

23. A method for deleting all or a portion of a gene, comprising:
introducing a first nucleic acid molecule encoding a chimeric endonuclease according to claim 1 into a cell comprising the gene under conditions causing expression of the chimeric endonuclease and cleavage of the gene;
introducing into the cell a second nucleic acid molecule comprising a region having a nucleotide sequence that has a high degree of sequence identity to the gene in the region of the cleavage site under conditions causing homologous recombination to occur between the second nucleic acid molecule and the gene, wherein the nucleotide sequence lacks the sequence of the gene adjacent to the cleavage site.

24. A method according to claim 23, wherein the region comprises 500 basepairs that are homologous to the gene.

25. A method according to claim 24, wherein the region comprises an altered sequence when compared to the gene of interest.

26. A method according to claim 25, wherein the region comprises one or more mutations that will result in changes to one or more amino acids in a protein encoded by the gene.

27. A method according to claim 23, wherein the chimeric endonuclease is transiently expressed in the cell.

28. A method according to claim 23, wherein the first nucleic acid molecule is mRNA.

29. A method according to claim 23, wherein the second nucleic acid molecule is a linear DNA molecule.

30. A method according to claim 23, wherein the cell is a plant cell.

31. A method for making a cell having an altered genome, comprising:
introducing into the cell a first nucleic acid molecule encoding a chimeric endonuclease according to claim 1 under conditions causing expression of the chimeric endonuclease and cleavage of the gene.

32. A method according to claim 31, wherein the altered genome comprises an inactivated gene.

33. A method according to claim 31, comprising:
introducing into the cell a second nucleic acid molecule comprising a region having a nucleotide sequence that has a high degree of sequence identity to the gene in the region of the cleavage site under conditions causing homologous recombination between the gene and the second nucleic acid, wherein the homologous region comprises an altered sequence when compared to the gene.

34. A method according to claim 33, wherein the region comprises 500 basepairs that are homologous to the gene.

35. A method according to claim 34, wherein the region comprises one or more mutations that will result in changes to one or more amino acids in a protein encoded by the gene.

36. A method according to claim 33, wherein the nucleotide sequence of the region lacks the sequence of the gene adjacent to the cleavage site.

37. A method according to claim 33, wherein the chimeric endonuclease is transiently expressed in the cell.

38. A method according to claim 33, wherein the first nucleic acid molecule is mRNA.

39. A method according to claim 34, wherein the second nucleic acid molecule is a linear DNA molecule.

40. A method according to claim 33, wherein the cell is a plant cell.

41. A nucleic acid substrate for the endonuclease as defined in claim 1, said substrate comprising a cleavage motif of the nuclease domain, a spacer that correlates with the linking domain and a binding site for the DNA-targeting domain.

42. A cell incorporating the substrate as defined in claim 41.

43. A kit comprising the nucleic acid molecule of claim 10 and the substrate of claim
